(19) United States
(12) Patent Application Publication
(10) Pub. No.: US 2025/0109201 A1
(43) Pub. Date: Apr. 3, 2025

(54) ANTI-ILT2 ANTIBODIES AND USES THEREOF

(71) Applicant: AGENUS INC., Lexington, MA (US)

(72) Inventors: Milan Blanusa, Lexington, MA (US); Kornelia Schultze, Lexington, MA (US); Andrea Claudia Schuster, Lexington, MA (US); Emmanuel Cyrille Pascal Briend, Lexington, MA (US); Olga Ignatovich, Lexington, MA (US); David Adam Savitsky, Lexington, MA (US); K. Mark Bushell, Lexington, MA (US); Beth Wensley, Lexington, MA (US); Claire Galand, Lexington, MA (US); Malgorzata Pupecka-Swider, Lexington, MA (US)

(21) Appl. No.: 18/827,368

(22) Filed: Sep. 6, 2024

Related U.S. Application Data
(63) Continuation of application No. PCT/US2023/063821, filed on Mar. 7, 2023.
(60) Provisional application No. 63/268,945, filed on Mar. 7, 2022.

(51) Int. Cl. C07K 16/28

(52) U.S. Cl. CPC C07K 16/2803; C07K 2317/33; C07K 2317/41; C07K 2317/52; C07K 2317/734; C07K 2317/76; C07K 2317/90

(57) ABSTRACT

The present disclosure provides antibodies and polypeptides that specifically bind to Ig-like transcript 2 (ILT2). Also provided are pharmaceutical compositions comprising these antibodies, nucleic acids encoding these antibodies, expression vectors and host cells for making these antibodies, and methods of treating a subject using these antibodies.

### RELATED APPLICATIONS

This application claims benefit to U.S. Provisional Application No. 63/268,945, filed on Mar. 7, 2022, the content of which is herein incorporated by reference in its entirety.

### SEQUENCE LISTING

The content of the electronically submitted Sequence Listing XML (Name: 198451_SL; Size: 59,428 bytes; Created: Mar. 1, 2023) is herein incorporated by reference in its entirety.

### FIELD

The present disclosure relates to antibodies that are specific for human Ig-like transcript 2 (ILT2), and methods of use thereof.

### BACKGROUND

Ig-like transcript 2 (ILT2) is an inhibitory receptor belonging to the type I transmembrane glycoproteins, which have four extracellular immunoglobulin-like domains (D1-D4), a transmembrane region, and an intracellular tail with four immunoreceptor tyrosine-based inhibitory motifs (ITIMs). ILT2 is expressed on a variety of immune cells, such as subpopulations of T cells, B cells, natural killer (NK) cells, myeloid-derived suppressive cells (MDSCs), dendritic cells (DCs), and monocytes/macrophages. Its ligands are both classical (HLA-A, -B, and -C) and non-classical MHC class I molecules, but the receptor binds to HLA-G with a three- to four-fold higher affinity than to classical MHC class I molecules. Engagement of ILT2 with HLA-G inhibits the function and activity of both innate and adaptive antitumor immune responses, promoting cancer cells to escape immune surveillance and anti-tumor immunity. The overexpression of HLA-G in solid tumors is associated with poor prognosis, tumor metastasis, and shorter disease-free survival, suggesting that blockade of the ILT2/HLA-G interaction could be efficacious in cancer therapy.

Thus, there is a need for therapies targeting ILT2.

### SUMMARY

The present disclosure provides antibodies and polypeptides that specifically bind to ILT2 (e.g., human ILT2). Also provided are pharmaceutical compositions comprising these antibodies, nucleic acids encoding these antibodies, expression vectors and host cells for making these antibodies, and methods of treating a subject using these antibodies. The antibodies disclosed herein are particularly advantageous in that they are potent blockers of the ILT2/HLA-G interaction and demonstrate high ex vivo potentiation of immune cell functional activity, compared to other ILT2 antibodies currently in clinical development. Applicant believes this will translate into superior efficacy in vivo.

In one aspect, the present disclosure provides an antibody that specifically binds human ILT2, the antibody comprising: a VH comprising the CDRH1, CDRH2, and CDRH3 amino acid sequences of the VH amino acid sequence set forth in SEQ ID NO: 1; and a VL comprising the CDRL1, CDRL2, and CDRL3 amino acid sequences of the VL amino acid sequence set forth in SEQ ID NO: 8.

In an embodiment, the antibody comprises the CDRH1, CDRH2, and CDRH3 amino acid sequences, respectively, set forth in SEQ ID NOs: 9, 10, and 11.

In an embodiment, the antibody comprises the CDRL1, CDRL2, and CDRL3 amino acid sequences, respectively, set forth in SEQ ID NOs: 15, 16, and 19.

In an embodiment, the antibody comprises the CDRL1, CDRL2, and CDRL3 amino acid sequences, respectively, set forth in SEQ ID NOs: 12, 16, and 17; 13, 16, and 17; 14, 16, and 17; 12, 16, and 18; 13, 16, and 18; or 14, 16, and 18.

In an embodiment, the antibody comprises the CDRH1, CDRH2, CDRH3, CDRL1, CDRL2, and CDRL3 amino acid sequences, respectively, set forth in SEQ ID NOs: 9, 10, 11, 12, 16, and 17; 9, 10, 11, 13, 16, and 17; 9, 10, 11, 14, 16, and 17; 9, 10, 11, 12, 16, and 18; 9, 10, 11, 13, 16, and 18; or 9, 10, 11, 14, 16, and 18.

In an embodiment, the antibody comprises the CDRH1, CDRH2, CDRH3, CDRL1, CDRL2, and CDRL3 amino acid sequences, respectively, set forth in SEQ ID NOs: 9, 10, 11, 13, 16, and 18.

In an embodiment, the antibody comprises the VH amino acid sequence of SEQ ID NO: 1.

In an embodiment, the antibody comprises a heavy chain constant region, optionally selected from the group consisting of human IgG₁, IgG₂, IgG₃, IgG₄, IgA₁, IgA₂, and IgM.

In an embodiment, the heavy chain constant region is a human IgG₄ heavy chain constant region comprising a P at position 228, numbered according to the EU numbering system.

In an embodiment, the antibody comprises a heavy chain constant region that is a variant of a wild-type heavy chain constant region, wherein the variant heavy chain constant region binds to an FcγR with lower affinity than the wild-type heavy chain constant region binds to the FcγR.

In an embodiment, the amino acid sequence of the heavy chain constant region comprises:
(a) one or more of the following amino acid mutations, according to the EU numbering system: L234A, L235A, L235E, N297A, N297Q, N297G, P329A, P329G, G236D, P238D, S239D, S267E, L328F, and L328E; or
(b) a set of amino acid mutations selected from the group consisting of: L234A and L235A; L234A and L235E; L234A, L235A, and L329A; or L234A, L235A, and P329G; S267E and L328F; P238D and L328E; P238D and one or more substitutions selected from the group consisting of E233D, G237D, H268D, P271G, and A330R; P238D, E233D, G237D, H268D, P271G, and A330R; G236D and S267E; S239D and S267E; V262E, S267E, and L328F; and V264E, S267E, and L328F, according to the EU numbering system.

In an embodiment, the antibody comprises a heavy chain constant region that is a variant of a wild-type heavy chain constant region, wherein the variant heavy chain constant region binds to an FcγR with higher affinity than the wild-type heavy chain constant region binds to the FcγR.

In an embodiment, the amino acid sequence of the heavy chain constant region comprises:
(a) one or more of the following amino acid mutations, according to the EU numbering system: G236A, S239D, F243L, T256A, K290A, R292P, S298A, Y300L, V305I, A330L, 1332E, E333A, K334A, A339T, and P396L; or
(b) a set of amino acid mutations selected from the group consisting of: S239D; T256A; K290A; S298A; 1332E; E333A; K334A; A339T; S239D and 1332E; S239D, A330L, and I332E; S298A, E333A, and K334A; G236A, S239D, and I332E; and F243L, R292P, Y300L, V305I, and P396L, according to the EU numbering system.

In an embodiment, the heavy chain constant region comprises the amino acid sequence of SEQ ID NO: 33, 34, 35, 36, 37, 38, or 39.

In an embodiment, the antibody comprises a heavy chain comprising the amino acid sequence of SEQ ID NO: 20, 21, 22, 23, 24, 25, or 26.

In an embodiment, the antibody comprises the VL amino acid sequence of SEQ ID NO: 8.

In an embodiment, the antibody comprises the VL amino acid sequence of SEQ ID NO: 2, 3, 4, 5, 6, or 7.

In an embodiment, the antibody comprises a light chain comprising the amino acid sequence of SEQ ID NO: 27, 28, 29, 30, 31, or 32.

In an embodiment, the VH and VL comprise the amino acid sequences, respectively, set forth in SEQ ID NOs: 1 and 5, 1 and 2, 1 and 3, 1 and 4, 1 and 6, or 1 and 7.

In an embodiment, the heavy chain and light chain comprise the amino acid sequences, respectively, set forth in SEQ ID NOs: 26 and 30, 26 and 27, 26 and 28, 26 and 29, 26 and 31, 26 and 32, 25 and 27, 25 and 28, 25 and 29, 25 and 30, 25 and 31, 25 and 32, 24 and 27, 24 and 28, 24 and 29, 24 and 30, 24 and 31, 24 and 32, 23 and 27, 23 and 28, 23 and 29, 23 and 30, 23 and 31, 23 and 32, 22 and 27, 22 and 28, 22 and 29, 22 and 30, 22 and 31, 22 and 32, 21 and 27, 21 and 28, 21 and 29, 21 and 30, 21 and 31, 21 and 32, 20 and 27, 20 and 28, 20 and 29, 20 and 30, 20 and 31, or 20 and 32.

In an embodiment, the antibody blocks binding of ILT2 to HLA-G, HLA-A, HLA-B and HLA-C; and/or the antibody blocks ILT2 mediated inhibition of FcγR signaling.

In one aspect, the present disclosure provides a polypeptide comprising a VH comprising the CDRH1, CDRH2, and CDRH3 amino acid sequences of the VH amino acid sequence set forth in SEQ ID NO: 1.

In an embodiment, the polypeptide comprises the CDRH1, CDRH2, and CDRH3 amino acid sequences, respectively, set forth in SEQ ID NOs: 9, 10, and 11.

In an embodiment, the VH comprises the amino acid sequence of SEQ ID NO: 1.

In an embodiment, the polypeptide comprises a heavy chain comprising the amino acid sequence of SEQ ID NO: 20, 21, 22, 23, 24, 25, or 26.

In one aspect, the present disclosure provides a polypeptide comprising a VL comprising the CDRL1, CDRL2, and CDRL3 amino acid sequences of the VL amino acid sequence set forth in SEQ ID NO: 8.

In an embodiment, the VL comprises the CDRL1, CDRL2, and CDRL3 amino acid sequences, respectively, set forth in SEQ ID NOs: 15, 16, and 19.

In an embodiment, the VL comprises the CDRL1, CDRL2, and CDRL3 amino acid sequences, respectively, set forth in SEQ ID NOs: 12, 16, and 17; 13, 16, and 17; 14, 16, and 17; 12, 16, and 18; 13, 16, and 18; or 14, 16, and 18.

In an embodiment, the VL comprises the amino acid sequence of SEQ ID NO: 8.

In an embodiment, the VL comprises the amino acid sequence of SEQ ID NO: 2, 3, 4, 5, 6, or 7.

In an embodiment, the polypeptide comprises a light chain comprising the amino acid sequence of SEQ ID NO: 27, 28, 29, 30, 31, or 32.

In an embodiment, an antibody or polypeptide disclosed herein is conjugated to a cytotoxic agent, cytostatic agent, toxin, radionuclide, or detectable label.

In one aspect, the present disclosure provides a polynucleotide encoding: a VH, a VL, a heavy chain, and/or a light chain of an antibody disclosed herein; or a polypeptide disclosed herein.

In one aspect, the present disclosure provides a vector comprising a polynucleotide disclosed herein.

In one aspect, the present disclosure provides a recombinant host cell comprising:
(a) a polynucleotide disclosed herein;
(b) a vector disclosed herein;
(c) a first polynucleotide encoding a heavy chain variable region or a heavy chain of an antibody disclosed herein, and a second polynucleotide encoding a light chain variable region or a light chain of an antibody disclosed herein;
(d) a first vector comprising a first polynucleotide encoding a heavy chain variable region or a heavy chain of an antibody disclosed herein, and a second vector comprising a second polynucleotide encoding a light chain variable region or a light chain of an antibody disclosed herein.

In one aspect, the present disclosure provides a pharmaceutical composition comprising an antibody disclosed herein, a polypeptide disclosed herein, a polynucleotide disclosed herein, a vector disclosed herein, a host cell disclosed herein, and a pharmaceutically acceptable carrier or excipient.

In one aspect, the present disclosure provides a method of producing an antibody, the method comprising culturing a host cell disclosed herein under suitable conditions such that the polynucleotide is expressed, and the antibody is produced.

In one aspect, the present disclosure provides a method of treating cancer in a subject, the method comprising administering to the subject an effective amount of an antibody disclosed herein, a polypeptide disclosed herein, a polynucleotide disclosed herein, a vector disclosed herein, a host cell disclosed herein, or a pharmaceutical composition disclosed herein.

In one aspect, the present disclosure provides use of an antibody disclosed herein, a polypeptide disclosed herein, a polynucleotide disclosed herein, a vector disclosed herein, a host cell disclosed herein, or a pharmaceutical composition disclosed herein, for the manufacture of a medicament for the treatment of cancer in a subject in need thereof.

In one aspect, the present disclosure provides an antibody disclosed herein, a polypeptide disclosed herein, a polynucleotide disclosed herein, a vector disclosed herein, a host cell disclosed herein, or a pharmaceutical composition disclosed herein, for use in medicine.

In one aspect, the present disclosure provides an antibody disclosed herein, a polypeptide disclosed herein, a polynucleotide disclosed herein, a vector disclosed herein, a host cell disclosed herein, or a pharmaceutical composition disclosed herein, for use in the treatment of cancer in a subject in need thereof.

### BRIEF DESCRIPTION OF THE DRAWINGS

FIG. 1 is a graph showing binding of anti-ILT2 antibody BA211 and sequence optimized variants BA212, BA216, BA213, BA214, and BA215 to CHO cells expressing relatively low levels of human ILT2. Binding is shown by the mean fluorescence intensity (MFI) as a function of antibody concentration in μg/mL.

FIG. 2 is a graph showing binding of anti-ILT2 antibody BA211 and sequence optimized variants BA212, BA216, BA213, BA214, and BA215 to Jurkat cells expressing human ILT2. Binding is shown by the mean fluorescence intensity (MFI) as a function of antibody concentration in μg/mL.

FIG. 3 is a graph showing binding of sequence-optimized variant anti-ILT2 antibodies formatted with different Fc backbones to CHO cells expressing relatively high levels of human ILT2. Binding is shown by the mean fluorescence intensity (MFI) as a function of antibody concentration in μg/mL.

FIG. 4A-FIG. 4B are graphs showing binding of sequence-optimized variant anti-ILT2 antibody BA252 and reference antibody 15G8 to CHO cells expressing relatively high (FIG. 4A) and low (FIG. 4B) levels of human ILT2. Binding is shown by the mean fluorescence intensity (MFI) as a function of antibody concentration in μg/mL. ***p<0.001 and ****p<0.0001

FIG. 5 is a graph showing the ability of BA211 to block the interaction between HLA-G and high ILT2-expressing CHO cells. Blocking is shown by % of maximum mean fluorescence intensity (MFI) of HLA-G-Fc-PE as a function of antibody concentration (μg/mL).

FIG. 6 is a graph showing the ability of sequence-optimized variant anti-ILT2 antibody BA252 to block the interaction between HLA-G and high ILT2-expressing CHO cells. Blocking is shown by % of maximum mean fluorescence intensity (MFI) of HLA-G-Fc-PE as a function of antibody concentration (μg/mL).

FIG. 7A-FIG. 7C are graphs showing the ability of sequence-optimized variant anti-ILT2 antibody BA252 to block the interaction between HLA-A (FIG. 7A), HLA-B (FIG. 7B), or HLA-C (FIG. 7C) and high ILT2-expressing CHO cells. Blocking is shown by % of maximum mean fluorescence intensity (MFI) of HLA-A*02:01, HLA-B*07:02 and HLA-C*07:02 pentamers, respectively, as a function of antibody concentration (μg/mL).

FIG. 8A-FIG. 8B are graphs showing the increase in NFAT-Luciferase signaling in ILT2 and CD16-expressing Jurkat reporter cells, co-incubated with Ramos cells expressing HLA-G in the presence of anti-CD20 antibody rituximab, and increasing concentrations of sequence-optimized variants of anti-ILT2 IgG₁ (FIG. 8A) and IgG₄ (FIG. 8B) antibodies. Signaling is shown by the luciferase activity in Relative Luminescence Units (RLU) as a function of antibody concentration (μg/mL).

FIG. 9A-FIG. 9B are graphs comparing the increase in NFAT-Luciferase signaling in ILT2 and CD16-expressing Jurkat reporter cells, co-incubated with Ramos cells expressing HLA-G in the presence of anti-CD20 antibody rituximab, and increasing concentrations of BA252 or reference antibody 15G8 (FIG. 9A). Blocking is shown by the luciferase activity in Relative Luminescence Units (RLU) as a function of antibody concentration (μg/mL). The EC50 of BA252 in this experiment was significantly lower than that of 15G8 (FIG. 9B). ***p<0.001

FIG. 10 is a graph showing binding of sequence-optimized variant anti-ILT2 antibody BA252 and control antibodies to complement component C1q. Binding is shown by optical density (OD) at 450 nm as a function of antibody concentration (μg/mL).

FIG. 11A-FIG. 11B are graphs showing the activation of primary immune cells in healthy donor PBMCs primed in the presence of BA252, reference ILT2 antibody 15G8, or isotype control. Activation of CD8+T, NKT, and NK cells is shown as a function of % CD25 expression for 3 donors. Donors were considered as “Responders” (FIG. 11A) if at least one of analyzed immune subpopulations responded to BA252 by ≥20% increase of CD25 surface expression compared to isotype control, otherwise they were considered “Non-Responders” (FIG. 11B).

FIG. 12A-FIG. 12B are graphs showing differences in surface expression of CD163 (FIG. 12A) or CD86/CD206 (FIG. 12B) on primary human macrophages (Mφ) and presented as % out of total population of CD33+ myeloid cells. Mφ were differentiated from purified human monocytes in presence of isotype control, BA252 or 15G8 antibodies, and left untreated or polarized into M2-like Mφ using conditioned media from HLA-G expressing JEG-3 cancer cells (Cancer cell CM) or IL-10+TGFβ cocktail. ***p<0.001, *p<0.05, ns=not significant.

FIG. 13A-FIG. 13F are a series of graphs showing binding of conjugated anti-ILT2 antibodies BA252-APC (FIG. 13A), VMP55-PE (FIG. 13B), 1Q-G2-APC (FIG. 13C), 292305-PE (FIG. 13D), GHI/75-PE (FIG. 13E) and 4F9-FITC (FIG. 13F) to CHO cells expressing relatively high human ILT2 levels, in the absence or the presence of unconjugated BA252. Binding is shown as the median fluorescence intensity (MFI) as a function of concentration of unconjugated BA252.

### DETAILED DESCRIPTION

The instant disclosure provides anti-ILT2 antibodies and polypeptides. Also provided are pharmaceutical compositions comprising these antibodies, nucleic acids encoding these antibodies, expression vectors and host cells for making these antibodies, and methods of treating a subject using these antibodies. The antibodies disclosed herein are particularly useful for treating cancer in a subject.

### Definitions

The expression “ILT2,” as used herein, refers to immunoglobulin-like transcript 2, which is also known as leukocyte immunoglobulin-like receptor subfamily B member 1 (LILRB1) or leukocyte immunoglobulin-like receptor 1 (LIR-1). The amino acid sequence of full-length human Ig-like transcript 2 can be found at accession number Q8NHL6 (UniProtKB). ILT2 is an inhibitory receptor for class I MHC antigens that recognizes a broad spectrum of HLA-A, HLA-B, HLA-C, HLA-G and HLA-F alleles. All references to proteins, polypeptides and protein fragments herein are intended to refer to the human version of the respective protein, polypeptide or protein fragment unless explicitly specified as being from a non-human species. Thus, the expression “ILT2” means human ILT2 unless specified as being from a non-human species, e.g., “mouse ILT2,” “monkey ILT2,” etc.

TABLE 1
Exemplary ILT-2 amino acid sequences.

| | SEQ | |
|---|---|---|
| Description | ID NO | Amino acid sequence |
| Human ILT2 | 43 | GHLPKPTLWAEPGSVITQGSPVTLRCQGGQETQEYRLYREKKTALWITRIPQEL |
| PE01 full | | VKKGQFPIPSITWEHAGRYRCYYGSDTAGRSESSDPLELVVTGAYIKPTLSAQP |
| length with | | SPVVNSGGNVILQCDSQVAFDGFSLCKEGEDEHPQCLNSQPHARGSSRAIFSVG |
| extracellular | | PVSPSRRWWYRCYAYDSNSPYEWSLPSDLLELLVLGVSKKPSLSVQPGPIVAPE |
| domain (ECD), | | ETLTLQCGSDAGYNRFVLYKDGERDFLQLAGAQPQAGLSQANFTLGPVSRSYGG |
| transmembrane | | QYRCYGAHNLSSEWSAPSDPLDILIAGQFYDRVSLSVQPGPTVASGENVTLLCQ |
| domain (TM), | | SQGWMQTFLLTKEGAADDPWRLRSTYQSQKYQAEFPMGPVTSAHAGTYRCYGSQ |
| and | | SSKPYLLTHPSDPLELVVSGPSGGPSSPTTGPTSTSGPEDQPLTPTGSDPQSGL |
| intracellular | | GRHLGVVIGILVAVILLLLLLLLLFLILRHRRQGKHWTSTQRKADFQHPAGAVG |
| domain | | PEPTDRGLQWRSSPAADAQEENLYAAVKHTQPEDGVEMDTRSPHDEDPQAVTYA |
| | | EVKHSRPRREMASPPSPLSGEFLDTKDRQAEEDRQMDTEAAASEAPQDVTYAQL |
| | | HSLTLRREATEPPPSQEGPSPAVPSIYATLAIH |
| Human ILT2 | 44 | GHLPKPTLWAEPGSVITQGSPVTLRCQGGQETQEYRLYREKKTALWITRIPQEL |
| PE01 ECD | | VKKGQFPIPSITWEHAGRYRCYYGSDTAGRSESSDPLELVVTGAYIKPTLSAQP |
| | | SPVVNSGGNVILQCDSQVAFDGFSLCKEGEDEHPQCLNSQPHARGSSRAIFSVG |
| | | PVSPSRRWWYRCYAYDSNSPYEWSLPSDLLELLVLGVSKKPSLSVQPGPIVAPE |
| | | ETLTLQCGSDAGYNRFVLYKDGERDFLQLAGAQPQAGLSQANFTLGPVSRSYGG |
| | | QYRCYGAHNLSSEWSAPSDPLDILIAGQFYDRVSLSVQPGPTVASGENVTLLCQ |
| | | SQGWMQTFLLTKEGAADDPWRLRSTYQSQKYQAEFPMGPVTSAHAGTYRCYGSQ |
| | | SSKPYLLTHPSDPLELVVSGPSGGPSSPTTGPTSTSGPEDQPLTPTGSDPQSGL |
| | | GRH |
| Human ILT2 | 45 | GHLPKPTLWAEPGSVITQGSPVTLRCQGGQETQEYRLYREKKTAPWITRIPQEL |
| PE02 ECD | | VKKGQFPIPSITWEHTGRYRCYYGSDTAGRSESSDPLELVVTGAYIKPTLSAQP |
| | | SPVVNSGGNVTLQCDSQVAFDGFILCKEGEDEHPQCLNSQPHARGSSRAIFSVG |
| | | PVSPSRRWWYRCYAYDSNSPYEWSLPSDLLELLVLGVSKKPSLSVQPGPIVAPE |
| | | ETLTLQCGSDAGYNRFVLYKDGERDFLQLAGAQPQAGLSQANFTLGPVSRSYGG |
| | | QYRCYGAHNLSSEWSAPSDPLDILIAGQFYDRVSLSVQPGPTVASGENVTLLCQ |
| | | SQGWMQTFLLTKEGAADDPWRLRSTYQSQKYQAEFPMGPVTSAHAGTYRCYGSQ |
| | | SSKPYLLTHPSDPLELVVSGPSGGPSSPTTGPTSTSGPEDQPLTPTGSDPQSGL |
| | | GRH |
| Human ILT2 | 46 | GHLPKPTLWAEPGSVITQGSPVTLRCQGGQETQEYRLYREKKTAPWITRIPQEL |
| PE03 ECD | | VKKGQFPIPSITWEHAGRYRCYYGSDTAGRSESSDPLELVVTGAYIKPTLSAQP |
| (Sino | | SPVVNSGGNVTLQCDSQVAFDGFILCKEGEDEHPQCLNSQPHARGSSRAIFSVG |
| biological, | | PVSPSRRWWYRCYAYDSNSPYEWSLPSDLLELLVLGVSKKPSLSVQPGPIVAPE |
| 16014-H08H) | | ETLTLQCGSDAGYNRFVLYKDGERDFLQLAGAQPQAGLSQANFTLGPVSRSYGG |
| | | QYRCYGAHNLSSEWSAPSDPLDILIAGQFYDRVSLSVQPGPTVASGENVTLLCQ |
| | | SQGWMQTFLLTKEGAADDPWRLRSTYQSQKYQAEFPMGPVTSAHAGTYRCYGSQ |
| | | SSKPYLLTHPSDPLELVVSGPSGGPSSPTTGPTSTSGPEDQPLTPTGSDPQSGL |
| | | GRH |
| Cynomolgus | 47 | GILPKPTLWAEPDRVITQGSPVTLRCQGNLEAREYHLYRERKSASWITLVRPEL |
| ILT2 | | VKKGQFPIPSTTWEHTGRYRCLYYSHSWWSEPSDPLELVVTGVYSKPTLSALPS |
| orthologue | | PVVASGGNVTLQCDSQLAFGGFILCKEGDEHPQCLNSQPSTRGASRAVFSVGPV |
| protein, variant | | SPSRRWSYRCYGYDSRSPSVWSLPSDLLELLVPGVSKKPSLSVQPGPVVAPGEK |
| 1 ECD | | LTLQCGSDAGYDRFVLYKEGERDFPQRSGRQPQAGLSQANFTLGPVRGSHGGQY |
| | | RCSGAHNLSSEWSAPSDPLDILIAGQIRARPSLSVQPGPTVASGENVTLLCQSQ |
| | | GWMDTFLLTKEGAADAPLRLKSKYQSHMYQAEFPMSPVTSAHAGTYRCYASFSS |
| | | NPYLLTHPSEPLELVVSGPEDQPLTPTGSAPQSGLGRHLG |
| Cynomolgus | 48 | GILPKPTLWAEPDRVITQGSPVTLRCQGNLEALGYHLYRERKSASWITSIRPEL |
| ILT2 | | VRKGQFPIPSITWEDAGRYRCLYYSHSWWSEHSDPLELVVTGTYSKPTLSALPS |
| orthologue | | PVVASGGNVTLQCDSQLAFGGFILCKEGDEHPQCLNSQPSTRGASRAVFSVGPV |
| protein, variant | | SPSRRWSYRCYGYDSHSPYVWSLPSDLLELLVPGVSNKPSLSVQPGPVVAPGEK |
| 2 ECD | | LTLQCGSDVGYDRFVLYKEGERDLLQRSGRQPQAGLSQANFSLGPVWGSHGGQY |
| | | RCSGAHNLSSEWSAPSDPLDILISRQTQARPSLSVQPGPMVASGENVTLLCQSQ |
| | | GWMDTFLLIKEGAADAPLRLKSKRRSYIYQAEFPMSPVTSTHAGTYRCYGSFSS |
| | | NPYLLTHPSEPLELVVSGPSESPSLPLTGPTPTPGPEDQSLNPTGSDPQSGLGR |
| | | HLG |
| Cynomolgus | 49 | GILPKPTLWAEPDRVITQGSPVTLRCQGNLEARGYHLYRERKSASWITLIRPEL |
| ILT2 | | VKKGQFPIPSITWEHAGRYRCQYSSRNSWSEPSDPLELVVTGAYSKPTLSALPS |
| orthologue | | PVVASGGSVTLQCVSQVAFDGFTLCKEGEDEHPQRLNCQSHARGWSWAVFSVGP |
| protein, variant | | VSPSRRWSYRCYSYDSRSPYVWSLPSDLLELLVPGVSKKPSLSVQPGPVVAPGE |
| 3 ECD | | SLTLQCGSDVGYDRFVLYKEGEGEFLQRPGRQPQAGLSQANFTLGPVRRSHGGQ |
| | | YRCSGAHNLSSEWSAPSDPLDILISEQFYDVVSLSVQPGPTVTSGENVTLLCQS |
| | | RAQFHTFLLTREGAAHPPLHLRSEHQAQQYQAEFPMGPVTSAHAGTYRCYSSLS |
| | | FNPYLLSHPSDPLELVVSGPSRSPSPPLTGPTPTPGLGRHLG |
| Rhesus ILT2 | 50 | GTFPKPTLWAEPGSMISKGSPVTLRCQGSLPVQDYRLQREKKTASWVRRIQQEL |
| orthologue | | VKKGYFAIASIAWEHTGQYRCQYYSHSWWSEPSDPLELVVTGAYSKPTLSALPS |
| protein, variant | | PVVASGGNVTLQCDSQVAFGGFVLCKEGEDEHPQCHSQPRTRGSSRAVFSVGPV |
| 1 ECD | | SPSRRWSYRCYGYELHYRYVWSLPSDLLELLVPGVSKKPSLSVQPGPVVAPGDK |
| | | LTLQCGSDAGYNRFALYKEGERDFLQRPGRQPQAGLSQANFLLGPVRRSHGGQY |
| | | RCSGAHNLSSEWSAPSDPLDILIAGQIRGRPSLLVQPGPTVVSGENVTLLCQSS |
| | | WQFHVFLLTQAGAADAHLHLRSMYKYPKYQAEFPMSPVTSAHAGTYRCYGSHSS |
| | | DSYLLSIPSDPLELVVSGPSGGPSSPTTGPTSTCGPEDQPLTPTGSDPQSGLGR |
| | | HLG |
| Rhesus ILT2 | 51 | GILPKPTLWAEPGSMISEGSPVTLRCQGSLQVQDYCLYREKKPASWVRQIRQEL |
| orthologue | | VKKGYFAIGFITWEHTGQYRCQYYNHSWWSEPSDPLELVVIGAYSKPTLSALPS |
| protein, variant | | PVVASGRNVTLQCDSQVAFDGFILCKEGEDEHPQRLNCQSHARGWSWAVFSVGP |
| 2 ECD | | VSPSHRWSYRCYGYISSDPYVWSLPSDLLELLVPGVSKKPSLSVQPGPVVARGD |
| | | KLTLQCGSDAGYDRFALYKEGEGDFLQRPGQQPQAGLSQANFLLGPVSRSHGGQ |
| | | YRCSGAHNLSSEWSAPSDPLDILIAGQIRGRPFLSVQPGPKVVSGENVTLLCQS |
| | | SWQFHAFLLTQAGAADAHLHLRSMHKYPKYQAEFPISPVTSAHAGTYRCYGSRS |
| | | SNPYLLSVPSDPLELVVSGPSGGPSSPTTGPTSTCGPEDQPLTPTGSAPQSGLG |
| | | RHLG |
| African Green | 52 | GTLPKPTLWAEPGSVISEGSPVTLRCQGSLQVQEYRLYREKNPASWVRRIPQEL |
| Monkey ILT2 | | VKKGYFPIASITWEHAGRYRCNYGSYIEGRSEPSDPLDLMVTGAYRKPTLSALP |
| ECD | | SPVVASGGNVSLQCVSQVAFDSFILCKEGEDEHPQRLNSHFHAHGWSWAVFSVG |
| | | PVSPSRRWSYRCYGYDSRSPYMWSLPSDLLELLVPGVSKKPSLSVQPGPVVALG |
| | | EKLTLQCGSDAGYNRFVLYKEGERDFLQRAGRQPQAGLSQANFPLGLVSRSHGG |
| | | QYRCYGAHNLSSEWSSPSDPLDILISGQFYVTVSLSVLPGPMVASGENVTLLCQ |
| | | SWWELHTFLLTKEGAAHPPPRLRSKHRAPKYQAEFPMGPVTSAHKGTYRCYGSH |
| | | SSNPYLLSLPSDPLELVVSGPSGSPSLPPTGSTPTPAGPEDQPLTPTGSDPQSG |
| | | LGRHLG |

As used herein, the terms “antibody” and “antibodies” include full-length antibodies, antigen-binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single-chain antibodies or single-chain Fvs (scFv), camelized antibodies, affibodies, Fab fragments, F(ab′)₂ fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA, or IgY), any class (e.g., IgG₁, IgG₂, IgG₃, IgG₄, IgA₁, or IgA₂), or any subclass (e.g., IgG₂a or IgG₂b) of immunoglobulin molecule. In certain embodiments, antibodies described herein are IgG antibodies, or a class (e.g., human IgG₁ or IgG₄) or subclass thereof. In a specific embodiment, the antibody is a humanized monoclonal antibody. In another specific embodiment, the antibody is a human monoclonal antibody.

“Multispecific antibodies” are antibodies (e.g., bispecific antibodies) that specifically bind to two or more different antigens or two or more different regions of the same antigen. Multispecific antibodies include bispecific antibodies that contain two different antigen-binding sites (exclusive of the Fc region). Multispecific antibodies can include, for example, recombinantly produced antibodies, human antibodies, humanized antibodies, resurfaced antibodies, chimeric antibodies, immunoglobulins, synthetic antibodies, tetrameric antibodies comprising two heavy chain and two light chain molecules, an antibody light chain monomer, heteroconjugate antibodies, linked single-chain antibodies or linked-single-chain Fvs (scFv), camelized antibodies, affibodies, linked Fab fragments, F(ab′)₂ fragments, chemically-linked Fvs, and disulfide-linked Fvs (sdFv). Multispecific antibodies can be of any type (e.g., IgG, IgE, IgM, IgD, IgA, or IgY), any class (e.g., IgG₁, IgG₂, IgG₃, IgG₄, IgA₁, or IgA₂), or any subclass (e.g., IgG₂a or IgG₂b) of immunoglobulin molecule. In certain embodiments, multispecific antibodies described herein are IgG antibodies, or a class (e.g., human IgG₁, IgG₂, or IgG₄) or subclass thereof.

As used herein, the term “CDR” or “complementarity determining region” means the noncontiguous antigen combining sites found within the variable regions of heavy and light chain polypeptides. These particular regions have been described by, for example, Kabat et al., J. Biol. Chem. 252, 6609-6616 (1977) and Kabat et al., Sequences of protein of immunological interest. (1991), by Chothia et al., J. Mol. Biol. 196:901-917 (1987), and by MacCallum et al., J. Mol. Biol. 262:732-745 (1996), all of which are herein incorporated by reference in their entireties, where the definitions include overlapping or subsets of amino acid residues when compared against each other. In certain embodiments, the term “CDR” is a CDR as defined by MacCallum et al., J. Mol. Biol. 262:732-745 (1996) and Martin A. “Protein Sequence and Structure Analysis of Antibody Variable Domains,” in Antibody Engineering, Kontermann and Dübel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of protein of immunological interest. (1991). In certain embodiments, heavy chain CDRs and light chain CDRs of an antibody are defined using different conventions. In certain embodiments, heavy chain CDRs and/or light chain CDRs are defined by performing structural analysis of an antibody and identifying residues in the variable region(s) predicted to make contact with an epitope region of a target molecule (e.g., human ILT2). CDRH1, CDRH2, and CDRH3 denote the heavy chain CDRs, and CDRL1, CDRL2, and CDRL3 denote the light chain CDRs.

As used herein, the terms “variable region” and “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable region are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In certain embodiments, the variable region is a primate (e.g., non-human primate) variable region. In certain embodiments, the variable region comprises rodent or murine CDRs and primate (e.g., non-human primate) framework regions (FRs).

As used herein, the terms “VH” and “VL” refer to antibody heavy and light chain variable regions, respectively, as described in Kabat et al., (1991) Sequences of Proteins of Immunological Interest (NIH Publication No. 91-3242, Bethesda), which is herein incorporated by reference in its entirety.

As used herein, the term “constant region” is common in the art. The constant region is an antibody portion, e.g., a carboxyl terminal portion of a light and/or heavy chain, which is not directly involved in binding of an antibody to antigen but which can exhibit various effector functions, such as interaction with an Fc receptor (e.g., Fc gamma receptor).

As used herein, the term “heavy chain” when used in reference to an antibody can refer to any distinct type, e.g., alpha (a), delta (8), epsilon (¿), gamma (γ), and mu (u), based on the amino acid sequence of the constant region, which give rise to IgA, IgD, IgE, IgG, and IgM classes of antibodies, respectively, including subclasses of IgG, e.g., IgG₁, IgG₂, IgG₃, and IgG₄.

As used herein, the term “light chain” when used in reference to an antibody can refer to any distinct type, e.g., kappa (κ) or lambda (λ), based on the amino acid sequence of the constant region. Light chain amino acid sequences are well known in the art. In specific embodiments, the light chain is a human light chain.

As used herein, the terms “specifically binds,” “specifically recognizes,” “immunospecifically binds,” and “immunospecifically recognizes” are analogous terms in the context of antibodies and refer to molecules that bind to an antigen (e.g., epitope or immune complex) as such binding is understood by one skilled in the art. For example, a molecule that specifically binds to an antigen can bind to other peptides or polypeptides, generally with lower affinity as determined by, e.g., immunoassays, BIAcore®, KinExA 3000 instrument (Sapidyne Instruments, Boise, ID), or other assays known in the art. In a specific embodiment, molecules that specifically bind to an antigen bind to the antigen with a K_{A} that is at least 2 logs (e.g., factors of 10), 2.5 logs, 3 logs, 4 logs, or greater than the K_{A} when the molecules bind non-specifically to another antigen.

As used herein, the term “affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein.

As used herein, the term “EU numbering system” refers to the EU numbering convention for the constant regions of an antibody, as described in Edelman, G. M. et al., Proc. Natl. Acad. USA, 63, 78-85 (1969) and Kabat et al, Sequences of Proteins of Immunological Interest, U.S. Dept. Health and Human Services, 5th edition, 1991, each of which is herein incorporated by reference in its entirety.

As used herein, the term “treat,” “treating,” and “treatment” refer to therapeutic or preventative measures described herein. The methods of “treatment” employ administration of an antibody to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “effective amount” in the context of the administration of a therapy to a subject refers to the amount of a therapy that achieves a desired prophylactic or therapeutic effect.

As used herein, the term “subject” includes any human or non-human animal. In certain embodiments, the subject is a human or non-human mammal. In certain embodiments, the subject is a human.

As used herein with respect to an antibody or polynucleotide, the term “isolated” refers to an antibody or polynucleotide that is separated from one or more contaminants (e.g., polypeptides, polynucleotides, lipids or carbohydrates, etc.) which are present in a natural source of the antibody or polynucleotide. All instances of “isolated antibodies” described herein are additionally contemplated as antibodies that may be, but need not be, isolated. All instances of “isolated polynucleotides” described herein are additionally contemplated as polynucleotides that may be, but need not be, isolated. All instances of “antibodies” described herein are additionally contemplated as antibodies that may be, but need not be, isolated. All instances of “polynucleotides” described herein are additionally contemplated as polynucleotides that may be, but need not be, isolated.

The determination of “percent identity” between two sequences (e.g., amino acid sequences or nucleic acid sequences) can be accomplished using a mathematical algorithm. A specific, non-limiting example of a mathematical algorithm utilized for the comparison of two sequences is the algorithm of Karlin S & Altschul S F (1990) PNAS 87:2264-2268, modified as in Karlin S & Altschul S F (1993) PNAS 90:5873-5877, each of which is herein incorporated by reference in its entirety. Such an algorithm is incorporated into the NBLAST and XBLAST programs of Altschul S F et al., (1990) J Mol Biol 215:403, which is herein incorporated by reference in its entirety. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25:3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

### Anti-ILT2 Antibodies

In one aspect, the instant disclosure provides antibodies that specifically bind to ILT2 (e.g., human ILT2). The amino acid sequences of exemplary antibodies are set forth in Table 2.

TABLE 2
Amino acid sequences of exemplary anti-ILT2 antibodies.

| | | | SEQ |
|---|---|---|---|
| Antibody | Description | Amino Acid Sequence | ID NO |
| BA210 BA220 | VH | QVQLVQSGAEVKKPGASVKVSCKASGYTENRYYMHWVRQAPGQGLEWMG | 1 |
| BA211 BA221 | | WINPYTGGTNYAQKFQGRVTMTRDTSISTAYMELSRLRSDDTAVYYCAS | |
| BA212 BA222 | | KQPSSDYWGQGTLVTVSS | |
| BA213 BA246 | | | |
| BA214 BA247 | | | |
| BA215 BA248 | | | |
| BA216 BA249 | | | |
| BA217 BA250 | | | |
| BA218 BA251 | | | |
| BA219 BA252 | | | |
| BA210 BA247 | VL | DIVMTQSPLSLPVTPGEPASISCRSSQSLLHSNGYNYLDWYLQKPGQSP | 2 |
| BA211 BA248 | | QLLIYLGSNRASGVPDRESGSGSGTDFTLKISRVEAEDVGVYYCMQASQ | |
| BA246 | | APWTFGQGTKVEIK | |
| BA212 | VL | DIVMTQSPLSLPVTPGEPASISCRSSQSLLHSNTYNYLDWYLQKPGQSP | 3 |
| | | QLLIYLGSNRASGVPDRFSGSGSGTDFTLKISRVEAEDVGVYYCMQASQ | |
| | | APWTFGQGTKVEIK | |
| BA213 | VL | DIVMTQSPLSLPVTPGEPASISCRSSQSLLHSNGYNYLDWYLQKPGQSP | 4 |
| | | QLLIYLGSNRASGVPDRESGSGSGTDETLKISRVEAEDVGVYYCLQASQ | |
| | | APWTFGQGTKVEIK | |
| BA214 BA249 | VL | DIVMTQSPLSLPVTPGEPASISCRSSQSLLHSNTYNYLDWYLQKPGQSP | 5 |
| BA217 BA251 | | QLLIYLGSNRASGVPDRESGSGSGTDFTLKISRVEAEDVGVYYCLQASQ | |
| BA219 BA252 | | APWTFGQGTKVEIK | |
| BA221 | | | |
| BA215 BA222 | VL | DIVMTQSPLSLPVTPGEPASISCRSSQSLLHSNYYNYLDWYLQKPGQSP | 6 |
| BA218 BA250 | | QLLIYLGSNRASGVPDRESGSGSGTDFTLKISRVEAEDVGVYYCLQASQ | |
| BA220 | | APWTFGQGTKVEIK | |
| BA216 | VL | DIVMTQSPLSLPVTPGEPASISCRSSQSLLHSNYYNYLDWYLQKPGQSP | 7 |
| | | QLLIYLGSNRASGVPDRFSGSGSGTDFTLKISRVEAEDVGVYYCMQASQ | |
| | | APWTFGQGTKVEIK | |
| Consensus | VL | DIVMTQSPLSLPVTPGEPASISCRSSQSLLHSNX₁YNYLDWYLQKPGQS | 8 |
| | | PQLLIYLGSNRASGVPDRESGSGSGTDFTLKISRVEAEDVGVYYCX₂QA | |
| | | SQAPWTFGQGTKVEIK | |
| | | X₁ is G, T, or Y | |
| | | X₂ is L or M | |
| BA210 BA220 | CDRH1 | RYYMH | 9 |
| BA211 BA221 | | | |
| BA212 BA222 | | | |
| BA213 BA246 | | | |
| BA214 BA247 | | | |
| BA215 BA248 | | | |
| BA216 BA249 | | | |
| BA217 BA250 | | | |
| BA218 BA251 | | | |
| BA219 BA252 | | | |
| BA210 BA220 | CDRH2 | WINPYTGGTNYAQKFQG | 10 |
| BA211 BA221 | | | |
| BA212 BA222 | | | |
| BA213 BA246 | | | |
| BA214 BA247 | | | |
| BA215 BA248 | | | |
| BA216 BA249 | | | |
| BA217 BA250 | | | |
| BA218 BA251 | | | |
| BA219 BA252 | | | |
| BA210 BA220 | CDRH3 | KQPSSDY | 11 |
| BA211 BA221 | | | |
| BA212 BA222 | | | |
| BA213 BA246 | | | |
| BA214 BA247 | | | |
| BA215 BA248 | | | |
| BA216 BA249 | | | |
| BA217 BA250 | | | |
| BA218 BA251 | | | |
| BA219 BA252 | | | |
| BA210 BA246 | CDRL 1 | RSSQSLLHSNGYNYLD | 12 |
| BA211 BA247 | | | |
| BA213 BA248 | | | |
| BA212 BA221 | CDRL 1 | RSSQSLLHSNTYNYLD | 13 |
| BA214 BA249 | | | |
| BA217 BA251 | | | |
| BA219 BA252 | | | |
| BA215 BA220 | CDRL 1 | RSSQSLLHSNYYNYLD | 14 |
| BA216 BA222 | | | |
| BA218 BA250 | | | |
| Consensus | CDRL 1 | RSSQSLLHSNX₁YNYLD | 15 |
| | | X₁ is G, T, or Y | |
| BA211 BA221 | | | |
| BA212 BA222 | | | |
| BA213 BA246 | | | |
| BA214 BA247 | | | |
| BA215 BA248 | | | |
| BA216 BA249 | | | |
| BA217 BA250 | | | |
| BA218 BA251 | | | |
| BA219 BA252 | | | |
| BA210 BA246 | CDRL3 | MQASQAPWT | 17 |
| BA211 BA247 | | | |
| BA212 BA248 | | | |
| BA216 | | | |
| BA213 BA221 | CDRL3 | LQASQAPWT | 18 |
| BA214 BA222 | | | |
| BA215 BA249 | | | |
| BA217 BA250 | | | |
| BA218 BA251 | | | |
| BA219 BA252 | | | |
| BA220 | | | |
| Consensus | CDRL3 | X₁QASQAPWT | 19 |
| | | X₁ is L or M | |
| BA210 | HC | QVQLVQSGAEVKKPGASVKVSCKASGYTFNRYYMHWVRQAPGQGLEWMG | 20 |
| BA249 | | WINPYTGGTNYAQKFQGRVTMTRDTSISTAYMELSRLRSDDTAVYYCAS | |
| BA250 | | KQPSSDYWGQGTLVTVSSASTKGPSVFPLAPSSKSTSGGTAALGCLVKD | |
| | | YFPEPVTVSWNSGALTSGVHTFPAVLQSSGLYSLSSVVTVPSSSLGTQT | |
| | | YICNVNHKPSNTKVDKRVEPKSCDKTHTCPPCPAPELLGGPSVELFPPK | |
| | | PKDTLMISRTPEVTCVVVDVSHEDPEVKENWYVDGVEVHNAKTKPREEQ | |
| | | YNSTYRVVSVLTVLHQDWLNGKEYKCKVSNKALPAPIEKTISKAKGQPR | |
| | | EPQVYTLPPSREEMTKNQVSLTCLVKGFYPSDIAVEWESNGQPENNYKT | |
| | | TPPVLDSDGSFFLYSKLTVDKSRWQQGNVFSCSVMHEALHNHYTQKSLS | |
| | | LSPGK | |
| BA211 | HC | QVQLVQSGAEVKKPGASVKVSCKASGYTENRYYMHWVRQAPGQGLEWMG | 21 |
| BA212 | | WINPYTGGTNYAQKFQGRVTMTRDTSISTAYMELSRLRSDDTAVYYCAS | |
| BA213 | | KQPSSDYWGQGTLVTVSSASTKGPSVFPLAPSSKSTSGGTAALGCLVKD | |
| BA214 | | YFPEPVTVSWNSGALTSGVHTFPAVLQSSGLYSLSSVVTVPSSSLGTQT | |
| BA215 | | YICNVNHKPSNTKVDKRVEPKSCDKTHTCPPCPAPELLGGPSVELFPPK | |
| BA216 | | PKDTLMISRTPEVTCVVVDVSHEDPEVKENWYVDGVEVHNAKTKPREEQ | |
| | | YASTYRVVSVLTVLHQDWLNGKEYKCKVSNKALPAPIEKTISKAKGQPR | |
| | | EPQVYTLPPSREEMTKNQVSLTCLVKGFYPSDIAVEWESNGQPENNYKT | |
| | | TPPVLDSDGSFFLYSKLTVDKSRWQQGNVFSCSVMHEALHNHYTQKSLS | |
| | | LSPGK | |
| BA217 | HC | QVQLVQSGAEVKKPGASVKVSCKASGYTENRYYMHWVRQAPGQGLEWMG | 22 |
| BA218 | | WINPYTGGTNYAQKFQGRVTMTRDTSISTAYMELSRLRSDDTAVYYCAS | |
| BA246 | | KQPSSDYWGQGTLVTVSSASTKGPSVFPLAPCSRSTSESTAALGCLVKD | |
| | | YFPEPVTVSWNSGALTSGVHTFPAVLQSSGLYSLSSVVTVPSSNFGTQT | |
| | | YTCNVDHKPSNTKVDKTVERKCCVECPPCPAPPVAGPSVELFPPKPKDT | |
| | | LMISRTPEVTCVVVDVSHEDPEVQFNWYVDGVEVHNAKTKPREEQENST | |
| | | FRVVSVLTVVHQDWLNGKEYKCKVSNKGLPAPIEKTISKTKGQPREPQV | |
| | | YTLPPSREEMTKNQVSLTCLVKGFYPSDIAVEWESNGQPENNYKTTPPM | |
| | | LDSDGSFFLYSKLTVDKSRWQQGNVFSCSVMHEALHNHYTQKSLSLSPG | |
| | | K | |
| BA219 | HC | QVQLVQSGAEVKKPGASVKVSCKASGYTFNRYYMHWVRQAPGQGLEWMG | 23 |
| BA220 | | WINPYTGGTNYAQKFQGRVTMTRDTSISTAYMELSRLRSDDTAVYYCAS | |
| BA247 | | KQPSSDYWGQGTLVTVSSASTKGPSVFPLAPSSRSTSESTAALGCLVKD | |
| | | YFPEPVTVSWNSGALTSGVHTFPAVLQSSGLYSLSSVVTVPSSNFGTQT | |
| | | YTCNVDHKPSNTKVDKTVERKCCVECPPCPAPPVAGPSVELFPPKPKDT | |
| | | LMISRTPEVTCVVVDVSHEDPEVQFNWYVDGVEVHNAKTKPREEQENST | |
| | | FRVVSVLTVVHQDWLNGKEYKCKVSNKGLPAPIEKTISKTKGQPREPQV | |
| | | YTLPPSREEMTKNQVSLTCLVKGFYPSDIAVEWESNGQPENNYKTTPPM | |
| | | LDSDGSFFLYSKLTVDKSRWQQGNVFSCSVMHEALHNHYTQKSLSLSPG | |
| | | K | |
| BA221 | HC | QVQLVQSGAEVKKPGASVKVSCKASGYTENRYYMHWVRQAPGQGLEWMG | 24 |
| BA222 | | WINPYTGGTNYAQKFQGRVTMTRDTSISTAYMELSRLRSDDTAVYYCAS | |
| BA248 | | KQPSSDYWGQGTLVTVSSASTKGPSVFPLAPCSRSTSESTAALGCLVKD | |
| | | YFPEPVTVSWNSGALTSGVHTFPAVLQSSGLYSLSSVVTVPSSSLGTKT | |
| | | YTCNVDHKPSNTKVDKRVESKYGPPCPPCPAPEFLGGPSVFLFPPKPKD | |
| | | TLMISRTPEVTCVVVDVSQEDPEVQFNWYVDGVEVHNAKTKPREEQENS | |
| | | TYRVVSVLTVLHQDWLNGKEYKCKVSNKGLPSSIEKTISKAKGQPREPQ | |
| | | VYTLPPSQEEMTKNQVSLTCLVKGFYPSDIAVEWESNGQPENNYKTTPP | |
| | | VLDSDGSFFLYSRLTVDKSRWQEGNVFSCSVMHEALHNHYTQKSLSLSL | |
| | | GK | |
| BA251 | HC | QVQLVQSGAEVKKPGASVKVSCKASGYTFNRYYMHWVRQAPGQGLEWMG | 25 |
| | | WINPYTGGTNYAQKFQGRVTMTRDTSISTAYMELSRLRSDDTAVYYCAS | |
| | | KQPSSDYWGQGTLVTVSSASTKGPSVFPLAPSSKSTSGGTAALGCLVKD | |
| | | YFPEPVTVSWNSGALTSGVHTFPAVLQSSGLYSLSSVVTVPSSSLGTQT | |
| | | YICNVNHKPSNTKVDKRVEPKSCDKTHTCPPCPAPELLGGPSVFLFPPK | |
| | | PKDTLMISRTPEVTCVVVDVSHEDPEVKENWYVDGVEVHNAKTKPREEQ | |
| | | YASTYRVVSVLTVLHQDWLNGKEYKCKVSNKALPAPIEKTISKAKGQPR | |
| | | EPQVYTLPPSREEMTKNQVSLTCLVKGFYPSDIAVEWESNGQPENNYKT | |
| | | TPPVLDSDGSFFLYSKLTVDKSRWQQGNVFSCSVMHEALHNHYTQKSLS | |
| | | LSPG | |
| BA252 | HC | QVQLVQSGAEVKKPGASVKVSCKASGYTFNRYYMHWVRQAPGQGLEWMG | 26 |
| | | WINPYTGGTNYAQKFQGRVTMTRDTSISTAYMELSRLRSDDTAVYYCAS | |
| | | KQPSSDYWGQGTLVTVSSASTKGPSVFPLAPCSRSTSESTAALGCLVKD | |
| | | YFPEPVTVSWNSGALTSGVHTFPAVLQSSGLYSLSSVVTVPSSSLGTKT | |
| | | YTCNVDHKPSNTKVDKRVESKYGPPCPPCPAPEFLGGPSVFLFPPKPKD | |
| | | TLMISRTPEVTCVVVDVSQEDPEVQFNWYVDGVEVHNAKTKPREEQENS | |
| | | TYRVVSVLTVLHQDWLNGKEYKCKVSNKGLPSSIEKTISKAKGQPREPQ | |
| | | VYTLPPSQEEMTKNQVSLTCLVKGFYPSDIAVEWESNGQPENNYKTTPP | |
| | | VLDSDGSFFLYSRLTVDKSRWQEGNVESCSVMHEALHNHYTQKSLSLSL | |
| | | G | |
| BA210 | LC | DIVMTQSPLSLPVTPGEPASISCRSSQSLLHSNGYNYLDWYLQKPGQSP | 27 |
| BA211 | | QLLIYLGSNRASGVPDRFSGSGSGTDFTLKISRVEAEDVGVYYCMQASQ | |
| BA246 | | APWTFGQGTKVEIKRTVAAPSVFIFPPSDEQLKSGTASVVCLLNNFYPR | |
| BA247 | | EAKVQWKVDNALQSGNSQESVTEQDSKDSTYSLSSTLTLSKADYEKHKV | |
| BA248 | | YACEVTHQGLSSPVTKSENRGEC | |
| BA212 | LC | DIVMTQSPLSLPVTPGEPASISCRSSQSLLHSNTYNYLDWYLQKPGQSP | 28 |
| | | QLLIYLGSNRASGVPDRFSGSGSGTDFTLKISRVEAEDVGVYYCMQASQ | |
| | | APWTFGQGTKVEIKRTVAAPSVFIFPPSDEQLKSGTASVVCLLNNFYPR | |
| | | EAKVQWKVDNALQSGNSQESVTEQDSKDSTYSLSSTLTLSKADYEKHKV | |
| | | YACEVTHQGLSSPVTKSENRGEC | |
| BA213 | LC | DIVMTQSPLSLPVTPGEPASISCRSSQSLLHSNGYNYLDWYLQKPGQSP | 29 |
| | | QLLIYLGSNRASGVPDRFSGSGSGTDFTLKISRVEAEDVGVYYCLQASQ | |
| | | APWTFGQGTKVEIKRTVAAPSVFIFPPSDEQLKSGTASVVCLLNNFYPR | |
| | | EAKVQWKVDNALQSGNSQESVTEQDSKDSTYSLSSTLTLSKADYEKHKV | |
| | | YACEVTHQGLSSPVTKSENRGEC | |
| BA214 BA249 | LC | DIVMTQSPLSLPVTPGEPASISCRSSQSLLHSNTYNYLDWYLQKPGQSP | 30 |
| BA217 BA251 | | QLLIYLGSNRASGVPDRESGSGSGTDFTLKISRVEAEDVGVYYCLQASQ | |
| BA219 BA252 | | APWTFGQGTKVEIKRTVAAPSVFIFPPSDEQLKSGTASVVCLLNNFYPR | |
| BA221 | | EAKVQWKVDNALQSGNSQESVTEQDSKDSTYSLSSTLTLSKADYEKHKV | |
| | | YACEVTHQGLSSPVTKSENRGEC | |
| BA215 | LC | DIVMTQSPLSLPVTPGEPASISCRSSQSLLHSNYYNYLDWYLQKPGQSP | 31 |
| BA218 | | QLLIYLGSNRASGVPDRESGSGSGTDFTLKISRVEAEDVGVYYCLQASQ | |
| BA220 | | APWTFGQGTKVEIKRTVAAPSVFIFPPSDEQLKSGTASVVCLLNNFYPR | |
| BA222 | | EAKVQWKVDNALQSGNSQESVTEQDSKDSTYSLSSTLTLSKADYEKHKV | |
| BA250 | | YACEVTHQGLSSPVTKSENRGEC | |
| BA21 | LC | DIVMTQSPLSLPVTPGEPASISCRSSQSLLHSNYYNYLDWYLQKPGQSP | 32 |
| | | QLLIYLGSNRASGVPDRESGSGSGTDFTLKISRVEAEDVGVYYCMQASQ | |
| | | APWTFGQGTKVEIKRTVAAPSVFIFPPSDEQLKSGTASVVCLLNNFYPR | |
| | | EAKVQWKVDNALQSGNSQESVTEQDSKDSTYSLSSTLTLSKADYEKHKV | |
| | | YACEVTHQGLSSPVTKSENRGEC | |

| | | |
|---|---|---|
| Human IgG1 heavy chain | ASTKGPSVFPLAPSSKSTSGGTAALGCLVKDYFPEPVTVSWNSGALTSG | 33 |
| constant region | VHTFPAVLQSSGLYSLSSVVTVPSSSLGTQTYICNVNHKPSNTKVDKRV | |
| | EPKSCDKTHTCPPCPAPELLGGPSVFLFPPKPKDTLMISRTPEVTCVVV | |
| | DVSHEDPEVKENWYVDGVEVHNAKTKPREEQYNSTYRVVSVLTVLHQDW | |
| | LNGKEYKCKVSNKALPAPIEKTISKAKGQPREPQVYTLPPSREEMTKNQ | |
| | VSLTCLVKGFYPSDIAVEWESNGQPENNYKTTPPVLDSDGSFFLYSKLT | |
| | VDKSRWQQGNVFSCSVMHEALHNHYTQKSLSLSPGK | |
| Human IgG1 heavy chain | ASTKGPSVFPLAPSSKSTSGGTAALGCLVKDYFPEPVTVSWNSGALTSG | 34 |
| constant region N297A | VHTFPAVLQSSGLYSLSSVVTVPSSSLGTQTYICNVNHKPSNTKVDKRV | |
| | EPKSCDKTHTCPPCPAPELLGGPSVFLFPPKPKDTLMISRTPEVTCVVV | |
| | DVSHEDPEVKFNWYVDGVEVHNAKTKPREEQYASTYRVVSVLTVLHQDW | |
| | LNGKEYKCKVSNKALPAPIEKTISKAKGQPREPQVYTLPPSREEMTKNQ | |
| | VSLTCLVKGFYPSDIAVEWESNGQPENNYKTTPPVLDSDGSFFLYSKLT | |
| | VDKSRWQQGNVFSCSVMHEALHNHYTQKSLSLSPGK | |
| Human IgG2 heavy chain | ASTKGPSVFPLAPCSRSTSESTAALGCLVKDYFPEPVTVSWNSGALTSG | 35 |
| constant region | VHTFPAVLQSSGLYSLSSVVTVPSSNFGTQTYTCNVDHKPSNTKVDKTV | |
| | ERKCCVECPPCPAPPVAGPSVFLFPPKPKDTLMISRTPEVTCVVVDVSH | |
| | EDPEVQFNWYVDGVEVHNAKTKPREEQFNSTFRVVSVLTVVHQDWLNGK | |
| | EYKCKVSNKGLPAPIEKTISKTKGQPREPQVYTLPPSREEMTKNQVSLT | |
| | CLVKGFYPSDIAVEWESNGQPENNYKTTPPMLDSDGSFFLYSKLTVDKS | |
| | RWQQGNVFSCSVMHEALHNHYTQKSLSLSPGK | |
| Human IgG2 heavy chain | ASTKGPSVFPLAPSSRSTSESTAALGCLVKDYFPEPVTVSWNSGALTSG | 36 |
| constant region C127S | VHTFPAVLQSSGLYSLSSVVTVPSSNFGTQTYTCNVDHKPSNTKVDKTV | |
| | ERKCCVECPPCPAPPVAGPSVFLFPPKPKDTLMISRTPEVTCVVVDVSH | |
| | EDPEVQFNWYVDGVEVHNAKTKPREEQFNSTFRVVSVLTVVHQDWLNGK | |
| | EYKCKVSNKGLPAPIEKTISKTKGQPREPQVYTLPPSREEMTKNQVSLT | |
| | CLVKGFYPSDIAVEWESNGQPENNYKTTPPMLDSDGSFFLYSKLTVDKS | |
| | RWQQGNVFSCSVMHEALHNHYTQKSLSLSPGK | |
| Human IgG4 heavy chain | ASTKGPSVFPLAPCSRSTSESTAALGCLVKDYFPEPVTVSWNSGALTSG | 37 |
| constant region S228P | VHTFPAVLQSSGLYSLSSVVTVPSSSLGTKTYTCNVDHKPSNTKVDKRV | |
| | ESKYGPPCPPCPAPEFLGGPSVFLFPPKPKDTLMISRTPEVTCVVVDVS | |
| | QEDPEVQFNWYVDGVEVHNAKTKPREEQFNSTYRVVSVLTVLHQDWLNG | |
| | KEYKCKVSNKGLPSSIEKTISKAKGQPREPQVYTLPPSQEEMTKNQVSL | |
| | TCLVKGFYPSDIAVEWESNGQPENNYKTTPPVLDSDGSFFLYSRLTVDK | |
| | SRWQEGNVFSCSVMHEALHNHYTQKSLSLSLGK | |
| Human IgG1 heavy chain | ASTKGPSVFPLAPSSKSTSGGTAALGCLVKDYFPEPVTVSWNSGALTSG | 38 |
| constant region N297A | VHTFPAVLQSSGLYSLSSVVTVPSSSLGTQTYICNVNHKPSNTKVDKRV | |
| without C-terminal | EPKSCDKTHTCPPCPAPELLGGPSVFLFPPKPKDTLMISRTPEVTCVVV | |
| lysine | DVSHEDPEVKFNWYVDGVEVHNAKTKPREEQYASTYRVVSVLTVLHQDW | |
| | LNGKEYKCKVSNKALPAPIEKTISKAKGQPREPQVYTLPPSREEMTKNQ | |
| | VSLTCLVKGFYPSDIAVEWESNGQPENNYKTTPPVLDSDGSFFLYSKLT | |
| | VDKSRWQQGNVFSCSVMHEALHNHYTQKSLSLSPG | |
| Human IgG4 heavy chain | ASTKGPSVFPLAPCSRSTSESTAALGCLVKDYFPEPVTVSWNSGALTSG | 39 |
| constant region S228P | VHTFPAVLQSSGLYSLSSVVTVPSSSLGTKTYTCNVDHKPSNTKVDKRV | |
| without C-terminal | ESKYGPPCPPCPAPEFLGGPSVFLFPPKPKDTLMISRTPEVTCVVVDVS | |
| lysine | QEDPEVQFNWYVDGVEVHNAKTKPREEQFNSTYRVVSVLTVLHQDWING | |
| | KEYKCKVSNKGLPSSIEKTISKAKGQPREPQVYTLPPSQEEMTKNQVSL | |
| | TCLVKGFYPSDIAVEWESNGQPENNYKTTPPVLDSDGSFFLYSRLTVDK | |
| | SRWQEGNVFSCSVMHEALHNHYTQKSLSLSLG | |
| Human kappa light | RTVAAPSVFIFPPSDEQLKSGTASVVCLLNNFYPREAKVQWKVDNALQS | 40 |
| chain constant region | GNSQESVTEQDSKDSTYSLSSTLTLSKADYEKHKVYACEVTHQGLSSPV | |
| | TKSENRGEC | |
| 15G8 VH (Reference | DVQLQGSGPGLVKPSETLSLTCSVTGYSITSGYYWNWIRQFPGKKLEWM | 41 |
| antibody) | GYISYDGSNNYNPSLKNRITISRDTSKNQFSLKLNSVTAADTATYYCAH | |
| | GYSYYYAMDAWGQGTSVTVSS | |
| 15G8 VL (Reference | DIQMTQSPSSLSASVGDRVTITCRTSQDISNYLNWYQQKPGKAVKLLIS | 42 |
| antibody) | YTSRLHSGVPSRFSGSGSGTDYTLTISSLQPEDFATYYCQQGNTLPTFG | |
| | QGTKLEIK | |

In certain embodiments, the instant disclosure provides an antibody that specifically binds to ILT2 (e.g., human ILT2), the antibody comprising a VH domain comprising one, two, or all three of the CDRs of a VH domain set forth in Table 2. In certain embodiments, the antibody comprises the CDRH1 of a VH domain set forth in Table 2. In certain embodiments, the antibody comprises the CDRH2 of a VH domain set forth in Table 2. In certain embodiments, the antibody comprises the CDRH3 of a VH domain set forth in Table 2.

In certain embodiments, the instant disclosure provides an antibody that specifically binds to ILT2 (e.g., human ILT2), the antibody comprising a VL domain comprising one, two, or all three of the CDRs of a VL domain disclosed in Table 2. In certain embodiments, the antibody comprises the CDRL1 of a VL domain set forth in Table 2. In certain embodiments, the antibody comprises the CDRL2 of a VL domain set forth in Table 2. In certain embodiments, the antibody comprises the CDRL3 of a VL domain set forth in Table 2.

The individual CDRs of an antibody disclosed herein can be determined according to any CDR numbering scheme known in the art.

In certain embodiments, one or more of the CDRs of an antibody disclosed herein can be determined according to Kabat et al., J. Biol. Chem. 252, 6609-6616 (1977) and Kabat et al., Sequences of protein of immunological interest (1991), each of which is herein incorporated by reference in its entirety.

In certain embodiments, the instant disclosure provides antibodies that specifically bind to ILT2 (e.g., human ILT2) and comprise CDRs of an antibody disclosed in Table 2 herein as determined by the Kabat numbering scheme.

In certain embodiments, one or more of the CDRs of an antibody disclosed herein can be determined according to the Chothia numbering scheme, which refers to the location of immunoglobulin structural loops (see, e.g., Chothia C & Lesk A M, (1987), J Mol Biol 196:901-917; Al-Lazikani B et al., (1997) J Mol Biol 273:927-948; Chothia C et al., (1992) J Mol Biol 227:799-817; Tramontano A et al., (1990) J Mol Biol 215 (1): 175-82; and U.S. Pat. No. 7,709,226, all of which are herein incorporated by reference in their entireties).

In certain embodiments, the instant disclosure provides antibodies that specifically bind to ILT2 (e.g., human ILT2) and comprise CDRs of an antibody disclosed in Table 2 herein, as determined by the Chothia numbering system.

In certain embodiments, one or more of the CDRs of an antibody disclosed herein can be determined according to MacCallum R M et al., (1996) J Mol Biol 262:732-745, herein incorporated by reference in its entirety. See also, e.g., Martin A. “Protein Sequence and Structure Analysis of Antibody Variable Domains,” in Antibody Engineering, Kontermann and Dübel, eds., Chapter 31, pp. 422-439, Springer-Verlag, Berlin (2001), herein incorporated by reference in its entirety.

In certain embodiments, the instant disclosure provides antibodies that specifically bind to ILT2 (e.g., human ILT2) and comprise CDRs of an antibody disclosed in Table 2 herein, as determined by the MacCallum numbering system.

In certain embodiments, the CDRs of an antibody disclosed herein can be determined according to the IMGT numbering system as described in: Lefranc M-P, (1999) The Immunologist 7:132-136; Lefranc M-P et al., (1999) Nucleic Acids Res 27:209-212, each of which is herein incorporated by reference in its entirety; and Lefranc M-P et al., (2009) Nucleic Acids Res 37: D1006-D1012.

In certain embodiments, the instant disclosure provides antibodies that specifically bind to ILT2 (e.g., human ILT2) and comprise CDRs of an antibody disclosed in Table 2 herein, as determined by the IMGT numbering system.

In certain embodiments, the CDRs of an antibody disclosed herein can be determined according to the AbM numbering scheme, which refers to AbM hypervariable regions, which represent a compromise between the Kabat CDRs and Chothia structural loops, and are used by Oxford Molecular's AbM antibody modeling software (Oxford Molecular Group, Inc.), herein incorporated by reference in its entirety.

In certain embodiments, the instant disclosure provides antibodies that specifically bind to ILT2 (e.g., human ILT2) and comprise CDRs of an antibody disclosed in Table 2 herein as determined by the AbM numbering scheme.

In certain embodiments, the CDRs of an antibody disclosed herein can be determined according to the AHo numbering system, as described in Honegger and Plückthun, A., J. Mol. Biol. 309:657-670 (2001), herein incorporated by reference in its entirety.

In certain embodiments, the instant disclosure provides antibodies that specifically bind to ILT2 (e.g., human ILT2) and comprise CDRs of an antibody disclosed in Table 2 herein, as determined by the AHo numbering system.

In certain embodiments, the individual CDRs of an antibody disclosed herein are each independently determined according to one of the Kabat, Chothia, MacCallum, IMGt, AHo, or AbM numbering schemes, or by structural analysis of the multispecific molecule, wherein the structural analysis identifies residues in the variable region(s) predicted to make contact with an epitope region of ILT2.

In certain embodiments, the instant disclosure provides an antibody that specifically bind ILT2 (e.g., human ILT2) comprising a VH comprising the CDRH1, CDRH2, and CDRH3 amino acid sequences of the VH amino acid sequence set forth in SEQ ID NO: 1, and a VL comprising the CDRL1, CDRL2, and CDRL3 amino acid sequences of the VL amino acid sequence set forth in SEQ ID NO: 8, wherein each CDR is independently determined according to one of the Kabat, Chothia, MacCallum, IMGt, AHo, or AbM numbering schemes, or by structural analysis of the multispecific molecule, wherein the structural analysis identifies residues in the variable region(s) predicted to make contact with an epitope region of ILT2 (e.g., human ILT2).

In certain embodiments, the instant disclosure provides an antibody that specifically binds to ILT2 (e.g., human ILT2), wherein the antibody comprises the CDRH1, CDRH2, CDRH3, CDRL1, CDRL2, and CDRL3 amino acid sequences of the VH and VL amino acid sequence set forth in SEQ ID NOs: 1 and 5, 1 and 2, 1 and 3, 1 and 4, 1 and 6, or 1 and 7, respectively.

In certain embodiments, the instant disclosure provides an antibody that specifically binds to ILT2 (e.g., human ILT2), wherein the antibody comprises a VH comprising the CDRH1, CDRH2 and CDRH3 amino acid sequences set forth in SEQ ID NOs: 9, 10, and 11, respectively.

In certain embodiments, the instant disclosure provides an antibody that specifically binds to ILT2 (e.g., human ILT2), wherein the antibody comprises a VL comprising the CDRL1, CDRL2, and CDRL3 amino acid sequences set forth in SEQ ID NOs: 15, 16, and 19, respectively.

In certain embodiments, the instant disclosure provides an antibody that specifically binds to ILT2 (e.g., human ILT2), wherein the antibody comprises a VL comprising the CDRL1, CDRL2, and CDRL3 amino acid sequences set forth in SEQ ID NOs: 12, 16, and 17; 13, 16, and 17; 14, 16, and 17; 12, 16, and 18; 13, 16, and 18; or 14, 16, and 18, respectively.

In certain embodiments, the instant disclosure provides an antibody that specifically binds to ILT2 (e.g., human ILT2), wherein the antibody comprises a VH comprising CDRH1, CDRH2, and CDRH3 regions, and a VL comprising CDRL1, CDRL2, and CDRL3 regions, wherein the CDRH1, CDRH2, CDRH3, CDRL1, CDRL2, and CDRL3 regions comprise the amino acid sequences set forth in SEQ ID NOs: 9, 10, 11, 12, 16, and 17; 9, 10, 11, 13, 16, and 17; 9, 10, 11, 14, 16, and 17; 9, 10, 11, 12, 16, and 18; 9, 10, 11, 13, 16, and 18; or 9, 10, 11, 14, 16, and 18, respectively.

In certain embodiments, the instant disclosure provides an antibody that specifically binds to ILT2 (e.g., human ILT2), wherein the antibody comprises a VH comprising CDRH1, CDRH2, and CDRH3 regions, and a VL comprising CDRL1, CDRL2, and CDRL3 regions, wherein the CDRH1, CDRH2, CDRH3, CDRL1, CDRL2, and CDRL3 regions comprise the amino acid sequences set forth in SEQ ID NOs: 9, 10, 11, 13, 16, and 18, respectively.

In certain embodiments, the instant disclosure provides an antibody that specifically binds to ILT2 (e.g., human ILT2) comprising a VH comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 1. In certain embodiments, the instant disclosure provides an antibody that specifically binds to ILT2 (e.g., human ILT2), comprising a VH comprising an amino acid sequence set forth in SEQ ID NO: 1. In certain embodiments, the amino acid sequence of the VH consists of the amino acid sequence set forth in SEQ ID NO: 1.

In certain embodiments, the instant disclosure provides an antibody that specifically binds to ILT2 (e.g., human ILT2), comprising a VL comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 8. In certain embodiments, the instant disclosure provides an antibody that specifically binds to ILT2 (e.g., human ILT2), comprising a VL comprising an amino acid sequence set forth in SEQ ID NO: 8. In certain embodiments, the amino acid sequence of the VL consists of the amino acid sequence set forth in SEQ ID NO: 8.

In certain embodiments, the instant disclosure provides an antibody that specifically binds to ILT2 (e.g., human ILT2), comprising a VL comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 2, 3, 4, 5, 6, or 7. In certain embodiments, the instant disclosure provides an antibody that specifically binds to ILT2 (e.g., human ILT2), comprising a VL comprising an amino acid sequence set forth in SEQ ID NO: 2, 3, 4, 5, 6, or 7. In certain embodiments, the amino acid sequence of the VL consists of the amino acid sequence set forth in SEQ ID NO: 2, 3, 4, 5, 6, or 7.

In certain embodiments, the instant disclosure provides an antibody that specifically binds to ILT2 (e.g., human ILT2), comprising a VH comprising an amino acid sequence that is at least 75%, 80%, 85%, 90%, 95%, or 100% (e.g., at least 86, 87, 88, 89, 90, 91, 92, 93, 94, 95, 96, 97, 98 or 99%) identical to the amino acid sequence set forth in SEQ ID NO: 1, and a VL comprising an amino acid sequence that is at least 75%, 80%, 85%, 90%, 95%, or 100% (e.g., at least 86, 87, 88, 89, 90, 91, 92, 93, 94, 95, 96, 97, 98, or 99%) identical to the amino acid sequence set forth in SEQ ID NO: 2, 3, 4, 5, 6, or 7. In certain embodiments, the instant disclosure provides an antibody that specifically binds to ILT2 (e.g., human ILT2), comprising a VH comprising an amino acid sequence of SEQ ID NO: 1, and a VL comprising an amino acid sequence of SEQ ID NO: 2, 3, 4, 5, 6, or 7. In certain embodiments, the amino acid sequence of the VH consists of the amino acid sequence set forth in SEQ ID NO: 1; and the amino acid sequence of the VL consists of the amino acid sequence set forth in SEQ ID NO: 2, 3, 4, 5, 6, or 7.

In certain embodiments, the instant disclosure provides an antibody that specifically binds to ILT2 (e.g., human ILT2), comprising the VH and VL amino acid sequences set forth in SEQ ID NOs: 1 and 5, 1 and 2, 1 and 3, 1 and 4, 1 and 6, or 1 and 7, respectively. In certain embodiments, the amino acid sequences of VH and VL consist of the amino acid sequences set forth in SEQ ID NOs: 1 and 5, 1 and 2, 1 and 3, 1 and 4, 1 and 6, or 1 and 7, respectively.

In certain embodiments, the instant disclosure provides an antibody that cross-competes for binding to ILT2 (e.g., human ILT2) with an antibody comprising the VH and VL amino acid sequences set forth in SEQ ID NOs: SEQ ID NOs: 1 and 5, 1 and 2, 1 and 3, 1 and 4, 1 and 6, or 1 and 7, respectively.

In certain embodiments, the instant disclosure provides an antibody that binds to the same or an overlapping epitope of ILT2 (e.g., an epitope of human ILT2) as an antibody described herein, e.g., an antibody comprising the VH and VL amino acid sequences set forth in SEQ ID NOs: 1 and 5, 1 and 2, 1 and 3, 1 and 4, 1 and 6, or 1 and 7, respectively.

In certain embodiments, the epitope of an antibody can be determined by, e.g., NMR spectroscopy, surface plasmon resonance (BIAcore®), X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giegé R et al., (1994) Acta Crystallogr D Biol Crystallogr 50 (Pt 4): 339-350; McPherson A (1990) Eur J Biochem 189:1-23; Chayen N E (1997) Structure 5:1269-1274; McPherson A (1976) J Biol Chem 251:6300-6303, all of which are herein incorporated by reference in their entireties). Antibody: antigen crystals may be studied using well known X-ray diffraction techniques and may be refined using computer software such as X-PLOR (Yale University, 1992, distributed by Molecular Simulations, Inc.; see, e.g., Meth Enzymol (1985) volumes 114 & 115, eds Wyckoff H W et al.; U.S. Patent Application No. 2004/0014194), and BUSTER (Bricogne G (1993) Acta Crystallogr D Biol Crystallogr 49 (Pt 1): 37-60; Bricogne G (1997) Meth Enzymol 276A: 361-423, ed Carter C W; Roversi P et al., (2000) Acta Crystallogr D Biol Crystallogr 56 (Pt 10): 1316-1323, all of which are herein incorporated by reference in their entireties). Mutagenesis mapping studies may be accomplished using any method known to one of skill in the art. See, e.g., Champe M et al., (1995) supra and Cunningham B C & Wells J A (1989) supra for a description of mutagenesis techniques, including alanine scanning mutagenesis techniques. In a specific embodiment, the epitope of an antibody is determined using alanine scanning mutagenesis studies. In addition, or antibodies that recognize and bind to the same or overlapping epitopes of ILT2 (e.g., human ILT2) can be identified using routine techniques such as an immunoassay, for example, by showing the ability of one antibody to block the binding of another antibody to a target antigen, i.e., a competitive binding assay. Competition binding assays also can be used to determine whether two antibodies have similar binding specificity for an epitope. Competitive binding can be determined in an assay in which the immunoglobulin under test inhibits specific binding of a reference antibody to a common antigen, such as ILT2 (e.g., human ILT2). Numerous types of competitive binding assays are known, for example: solid phase direct or indirect radioimmunoassay (RIA), solid phase direct or indirect enzyme immunoassay (EIA), sandwich competition assay (see Stahli C et al., (1983) Methods Enzymol 9:242-253); solid phase direct biotin-avidin EIA (see Kirkland T N et al., (1986) J Immunol 137:3614-9); solid phase direct labeled assay, solid phase direct labeled sandwich assay (see Harlow E & Lane D, (1988) Antibodies: A Laboratory Manual, Cold Spring Harbor Press); solid phase direct label RIA using I-125 label (see Morel G A et al., (1988) Mol Immunol 25 (1): 7-15); solid phase direct biotin-avidin EIA (see Cheung R C et al., (1990) Virology 176:546-52); and direct labeled RIA (see Moldenhauer G et al., (1990) Scand J Immunol 32:77-82), all of which are herein incorporated by reference in their entireties. Typically, such an assay involves the use of purified antigen (e.g., ILT2, such as human ILT2) bound to a solid surface or cells bearing either of these, an unlabeled test immunoglobulin and a labeled reference immunoglobulin. Competitive inhibition can be measured by determining the amount of label bound to the solid surface or cells in the presence of the test immunoglobulin. Usually, the test immunoglobulin is present in excess. Usually, when a competing antibody is present in excess, it will inhibit specific binding of a reference or antibody to a common antigen by at least 50-55%, 55-60%, 60-65%, 65-70%, 70-75% or more. A competition binding assay can be configured in a large number of different formats using either labeled antigen or labeled antibody. In a common version of this assay, the antigen is immobilized on a 96-well plate. The ability of unlabeled antibodies to block the binding of labeled antibodies to the antigen is then measured using radioactive or enzyme labels. For further details see, for example, Wagener C et al., (1983) J Immunol 130:2308-2315; Wagener C et al., (1984) J Immunol Methods 68:269-274; Kuroki M et al., (1990) Cancer Res 50:4872-4879; Kuroki M et al., (1992) Immunol Invest 21:523-538; Kuroki M et al., (1992) Hybridoma 11:391-407 and Antibodies: A Laboratory Manual, Ed Harlow E & Lane D editors supra, pp. 386-389, all of which are herein incorporated by reference in their entireties.

In certain embodiments, the instant disclosure provides an antibody that specifically binds to ILT2 (e.g., human ILT2), the antibody comprising a heavy chain comprising the amino acid sequence set forth in SEQ ID NO: 20, 21, 22, 23, 24, 25, or 26. In certain embodiments, the amino acid sequence of the heavy chain consists of the amino acid sequence set forth in SEQ ID NO: 20, 21, 22, 23, 24, 25, or 26.

In certain embodiments, the instant disclosure provides an antibody that specifically binds to ILT2 (e.g., human ILT2), the antibody comprising a light chain comprising the amino acid sequence set forth in SEQ ID NO: 27, 28, 29, 30, 31, or 32. In certain embodiments, the amino acid sequence of the light chain consists of an amino acid sequence selected from the group consisting of SEQ ID NO: 27, 28, 29, 30, 31, or 32.

In certain embodiments, the instant disclosure provides an antibody that specifically binds to ILT2 (e.g., human ILT2), comprising the heavy chain and light chain, wherein the heavy chain and light chain comprise the amino acid sequences of SEQ ID NOs: 26 and 30, 26 and 27, 26 and 28, 26 and 29, 26 and 31, 26 and 32, 25 and 27, 25 and 28, 25 and 29, 25 and 30, 25 and 31, 25 and 32, 24 and 27, 24 and 28, 24 and 29, 24 and 30, 24 and 31, 24 and 32, 23 and 27, 23 and 28, 23 and 29, 23 and 30, 23 and 31, 23 and 32, 22 and 27, 22 and 28, 22 and 29, 22 and 30, 22 and 31, 22 and 32, 21 and 27, 21 and 28, 21 and 29, 21 and 30, 21 and 31, 21 and 32, 20 and 27, 20 and 28, 20 and 29, 20 and 30, 20 and 31, or 20 and 32, respectively.

In certain embodiments, the amino acid sequences of the heavy chain and light chain consist of amino acid sequences selected from the groups consisting of SEQ ID NOs: 26 and 30, 26 and 27, 26 and 28, 26 and 29, 26 and 31, 26 and 32, 25 and 27, 25 and 28, 25 and 29, 25 and 30, 25 and 31, 25 and 32, 24 and 27, 24 and 28, 24 and 29, 24 and 30, 24 and 31, 24 and 32, 23 and 27, 23 and 28, 23 and 29, 23 and 30, 23 and 31, 23 and 32, 22 and 27, 22 and 28, 22 and 29, 22 and 30, 22 and 31, 22 and 32, 21 and 27, 21 and 28, 21 and 29, 21 and 30, 21 and 31, 21 and 32, 20 and 27, 20 and 28, 20 and 29, 20 and 30, 20 and 31, or 20 and 32, respectively.

The anti-ILT2 antigen-binding molecules of the present disclosure can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bispecific or a multispecific antibody with a second or additional binding specificity.

In certain embodiments, the antibody disclosed herein is conjugated to a cytotoxic agent, cytostatic agent, toxin, radionuclide, or detectable label. In certain embodiments, the cytotoxic agent is able to induce death or destruction of a cell in contact therewith. In certain embodiments, the cytostatic agent is able to prevent or substantially reduce proliferation and/or inhibits the activity or function of a cell in contact therewith. In certain embodiments, the cytotoxic agent or cytostatic agent is a chemotherapeutic agent. In certain embodiments, the radionuclide is selected from the group consisting of the isotopes ³H, ¹⁴C, ³²P, ³⁵S, ³⁶Cl, ⁵¹Cr, ⁵⁷Co, ⁵⁸Co, ⁵⁹Fe, ⁶⁷Cu, ⁹⁰Y, ⁹⁹Tc, ¹¹¹In, ¹¹⁷Lu, ¹²¹I, ¹²⁴I, ¹²⁵I, ¹³¹I, ¹⁹⁸Au, ²¹¹At, ²¹³Bi, ²²⁵Ac, and ¹⁸⁶Re. In certain embodiments, the detectable label comprises a fluorescent moiety or a click chemistry handle.

Any immunoglobulin (Ig) constant region can be used in the antibodies disclosed herein. In certain embodiments, the Ig region is a human IgG, IgE, IgM, IgD, IgA, or IgY immunoglobulin molecule, any class (e.g., IgG₁, IgG₂, IgG₃, IgG₄, IgA₁, and IgA₂), or any subclass (e.g., IgG₂a and IgG₂b) of immunoglobulin molecule.

In certain embodiments, the instant disclosure provides an antibody that specifically binds to ILT2 (e.g., human ILT2), the antibody comprising a heavy chain constant region, optionally selected from the group consisting of human IgG₁, IgG₂, IgG₃, IgG₄, IgA₁, IgA₂, and IgM.

In certain embodiments, the instant disclosure provides an antibody that specifically binds to ILT2 (e.g., human ILT2), the antibody comprising a heavy chain constant region that is a variant of a wild-type heavy chain constant region, wherein the variant heavy chain constant region binds to an FcγR with lower affinity than the wild-type heavy chain constant region binds to the FcγR.

In certain embodiments, the instant disclosure provides an antibody that specifically binds to ILT2 (e.g., human ILT2), the antibody comprising a heavy chain constant region comprising the amino acid sequence of SEQ ID NO: 33, 34, 35, 36, 37, 38, or 39. In certain embodiments, the instant disclosure provides an antibody that specifically binds to ILT2 (e.g., human ILT2), the antibody comprising a heavy chain constant region consisting of the amino acid sequence of SEQ ID NO: 33, 34, 35, 36, 37, 38, or 39.

In certain embodiments, one, two, or more mutations (e.g., amino acid substitutions) are introduced into an Fc region (e.g., a CH2 domain (residues 231-340 of human IgG₁)) and/or a CH3 domain (residues 341-447 of human IgG₁, numbered according to the EU numbering system) and/or a hinge region (residues 216-230, numbered according to the EU numbering system) of an antibody described herein, to alter one or more functional properties of the antibody, such as serum half-life, complement fixation, Fc receptor binding, and/or antigen-dependent cellular cytotoxicity.

In certain embodiments, one, two, or more mutations (e.g., amino acid substitutions) are introduced into the hinge region of an antibody described herein, such that the number of cysteine residues in the hinge region is altered (e.g., increased or decreased) as described in, e.g., U.S. Pat. No. 5,677,425, herein incorporated by reference in its entirety. The number of cysteine residues in the hinge region may be altered to, e.g., facilitate assembly of the light and heavy chains, or to alter (e.g., increase or decrease) the stability of the antibody.

In a specific embodiment, one, two, or more amino acid mutations (e.g., substitutions, insertions, or deletions) are introduced into an IgG constant region, or FcRn-binding fragment thereof (preferably an Fc or hinge-Fc fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745, all of which are herein incorporated by reference in their entireties, for examples of mutations that will alter (e.g., decrease or increase) the half-life of an antibody in vivo. In certain embodiments, one, two or more amino acid mutations (e.g., substitutions, insertions, or deletions) are introduced into an IgG constant region, or FcRn-binding fragment thereof (preferably an Fc or hinge-Fc fragment) to decrease the half-life of the antibody in vivo. In other embodiments, one, two or more amino acid mutations (e.g., substitutions, insertions, or deletions) are introduced into an IgG constant region, or FcRn-binding fragment thereof (preferably an Fc or hinge-Fc fragment) to increase the half-life of the antibody in vivo. In a specific embodiment, the antibodies may have one or more amino acid mutations (e.g., substitutions) in the second constant (CH2) domain (residues 231-340 of human IgG₁) and/or the third constant (CH3) domain (residues 341-447 of human IgG₁), numbered according to the EU numbering system. In a specific embodiment, the constant region of the IgG₁ of antibody described herein comprises a methionine (M) to tyrosine (Y) substitution in position 252, a serine(S) to threonine (T) substitution in position 254, and a threonine (T) to glutamic acid (E) substitution in position 256, numbered according to the EU numbering system. See U.S. Pat. No. 7,658,921, which is herein incorporated by reference in its entirety. This type of mutant IgG, referred to as “YTE mutant” has been shown to display fourfold increased half-life as compared to wild-type versions of the same antibody (see Dall'Acqua W F et al., (2006) J Biol Chem 281:23514-24, which is herein incorporated by reference in its entirety). In certain embodiments, an antibody comprises an IgG constant region comprising one, two, three or more amino acid substitutions of amino acid residues at positions 251-257, 285-290, 308-314, 385-389, and 428-436, numbered according to the EU numbering system.

In certain embodiments, one, two, or more mutations (e.g., amino acid substitutions) are introduced into an Fc region (e.g., a CH2 domain (residues 231-340 of human IgG₁)) and/or a CH3 domain (residues 341-447 of human IgG₁, numbered according to the EU numbering system) and/or a hinge region (residues 216-230, numbered according to the EU numbering system) of an antibody described herein, to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109:6181-6186, U.S. Pat. No. 6,737,056, and International Publication Nos. WO 02/060919; WO 98/23289; and WO 97/34631, all of which are herein incorporated by reference in their entireties.

In certain embodiments, the antibody comprises a heavy chain constant region that is a variant of a wild-type heavy chain constant region, wherein the variant heavy chain constant region binds to FcγRIIB with higher affinity than the wild-type heavy chain constant region binds to FcγRIIB. In certain embodiments, the variant heavy chain constant region is a variant human heavy chain constant region, e.g., a variant human IgG₁, a variant human IgG₂, or a variant human IgG₄ heavy chain constant region. In certain embodiments, the variant human IgG heavy chain constant region comprises one or more of the following amino acid mutations, according to the EU numbering system: G236D, P238D, S239D, S267E, L328F, and L328E. In certain embodiments, the variant human IgG heavy chain constant region comprises a set of amino acid mutations selected from the group consisting of: S267E and L328F; P238D and L328E; P238D and one or more substitutions selected from the group consisting of E233D, G237D, H268D, P271G, and A330R; P238D, E233D, G237D, H268D, P271G, and A330R; G236D and S267E; S239D and S267E; V262E, S267E, and L328F; and V264E, S267E, and L328F, according to the EU numbering system. In certain embodiments, the FcγRIIB is expressed on a cell selected from the group consisting of macrophages, monocytes, B cells, dendritic cells, endothelial cells, and activated T cells.

In a further embodiment, one, two, or more amino acid substitutions are introduced into an IgG constant region Fc region to alter the effector function(s) of the antibody. For example, one or more amino acids selected from amino acid residues 234, 235, 236, 237, 239, 243, 267, 292, 297, 300, 318, 320, 322, 328, 330, 332, and 396, numbered according to the EU numbering system, can be replaced with a different amino acid residue such that the antibody has an altered affinity for an effector ligand but retains the antigen-binding ability of the parent antibody. The effector ligand to which affinity is altered can be, for example, an Fc receptor or the Cl component of complement. This approach is described in further detail in U.S. Pat. Nos. 5,624,821 and 5,648,260, each of which is herein incorporated by reference in its entirety. In certain embodiments, the deletion or inactivation (through point mutations or other means) of a constant region domain may reduce Fc receptor binding of the circulating antibody thereby increasing tumor localization. See, e.g., U.S. Pat. Nos. 5,585,097 and 8,591,886, each of which is herein incorporated by reference in its entirety, for a description of mutations that delete or inactivate the constant region and thereby increase tumor localization. In certain embodiments, one or more amino acid substitutions may be introduced into the Fc region of an antibody described herein to remove potential glycosylation sites on the Fc region, which may reduce Fc receptor binding (see, e.g., Shields R L et al., (2001) J Biol Chem 276:6591-604, which is herein incorporated by reference in its entirety). In various embodiments, one or more of the following mutations in the constant region of an antibody described herein may be made: an N297A substitution; an N297Q substitution; an L234A substitution; an L234F substitution; an L235A substitution; an L235F substitution; an L235V substitution; an L237A substitution; an S239D substitution; an E233P substitution; an L234V substitution; a C236 deletion; a P238A substitution; an F243L substitution; a D265A substitution; an S267E substitution; an L328F substitution; an R292P substitution; a Y300L substitution; an A327Q substitution; a P329A substitution; an A330L substitution; an 1332E substitution; or a P396L substitution, numbered according to the EU numbering system.

In certain embodiments, a mutation selected from the group consisting of D265A, P329A, and a combination thereof, numbered according to the EU numbering system, may be made in the constant region of an antibody described herein. In certain embodiments, a mutation selected from the group consisting of L235A, L237A, and a combination thereof, numbered according to the EU numbering system, may be made in the constant region of an antibody described herein. In certain embodiments, a mutation selected from the group consisting of S267E, L328F, and a combination thereof, numbered according to the EU numbering system, may be made in the constant region of an antibody described herein. In certain embodiments, a mutation selected from the group consisting of S239D, 1332E, optionally A330L, and a combination thereof, numbered according to the EU numbering system, may be made in the constant region of an antibody described herein. In certain embodiments, a mutation selected from the group consisting of L235V, F243L, R292P, Y300L, P396L, and a combination thereof, numbered according to the EU numbering system, may be made in the constant region of an antibody described herein. In certain embodiments, a mutation selected from the group consisting of S267E, L328F, and a combination thereof, numbered according to the EU numbering system, may be made in the constant region of an antibody described herein.

In a specific embodiment, an antibody described herein comprises the constant region of an IgG₁ with an N297Q or N297A amino acid substitution, numbered according to the EU numbering system. In certain embodiments, an antibody described herein comprises the constant region of an IgG₁ with a mutation selected from the group consisting of D265A, P329A, and a combination thereof, numbered according to the EU numbering system. In another embodiment, an antibody described herein comprises the constant region of an IgG₁ with a mutation selected from the group consisting of L234A, L235A, and a combination thereof, numbered according to the EU numbering system. In another embodiment, an antibody described herein comprises the constant region of an IgG₁ with a mutation selected from the group consisting of L234F, L235F, N297A, and a combination thereof, numbered according to the EU numbering system. In certain embodiments, amino acid residues in the constant region of an antibody described herein in the positions corresponding to positions L234, L235, and D265 in a human IgG₁ heavy chain, numbered according to the EU numbering system, are not L, L, and D, respectively. This approach is described in detail in International Publication No. WO 14/108483, which is herein incorporated by reference in its entirety. In certain embodiments, the amino acids corresponding to positions L234, L235, and D265 in a human IgG₁ heavy chain are F, E, and A; or A, A, and A, respectively, numbered according to the EU numbering system.

In certain embodiments, one or more amino acids selected from amino acid residues 329, 331, and 322 in the constant region of an antibody described herein, numbered according to the EU numbering system, can be replaced with a different amino acid residue such that the antibody has altered C1q binding and/or reduced or abolished complement dependent cytotoxicity (CDC). This approach is described in further detail in U.S. Pat. No. 6,194,551 (Idusogie et al.), which is herein incorporated by reference in its entirety. In certain embodiments, one or more amino acid residues within amino acid positions 231 to 238 in the N-terminal region of the CH2 domain of an antibody described herein are altered to thereby alter the ability of the antibody to fix complement, numbered according to the EU numbering system. This approach is described further in International Publication No. WO 94/29351, which is herein incorporated by reference in its entirety. In certain embodiments, the Fc region of an antibody described herein is modified to increase the ability of the antibody to mediate antibody dependent cellular cytotoxicity (ADCC) and/or to increase the affinity of the antibody for an Fcγ receptor by mutating one or more amino acids (e.g., introducing amino acid substitutions) at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 328, 329, 330, 331, 333, 334, 335, 337, 338, 340, 360, 373, 376, 378, 382, 388, 389, 398, 414, 416, 419, 430, 434, 435, 437, 438, or 439, numbered according to the EU numbering system. This approach is described further in International Publication No. WO 00/42072, which is herein incorporated by reference in its entirety.

In certain embodiments, an antibody described herein comprises a modified constant region of an IgG₁, wherein the modification increases the ability of the antibody to mediate antibody dependent cellular cytotoxicity (ADCC). In certain embodiments, 0.1, 1, or 10 μg/mL of the antibody is capable of inducing cell death of at least 20%, 25%, 30%, 35%, 40%, 45%, 50%, 55%, or 60% of ILT2-expressing cells within 1, 2, or 3 hours, as assessed by methods described herein and/or known to a person of skill in the art. In certain embodiments, the modified constant region of an IgG₁ comprises S239D and 1332E substitutions, numbered according to the EU numbering system. In certain embodiments, the modified constant region of an IgG₁ comprises S239D, A330L, and I332E substitutions, numbered according to the EU numbering system. In certain embodiments, the modified constant region of an IgG₁ comprises L235V, F243L, R292P, Y300L, and P396L substitutions, numbered according to the EU numbering system. In certain embodiments, the antibody is capable of inducing cell death in effector T cells and Tregs, wherein the percentage of Tregs that undergo cell death is higher than the percentage of effector T cells that undergo cell death by at least 1.2 fold, 1.3 fold, 1.4 fold, 1.5 fold, 1.6 fold, 1.7 fold, 1.8 fold, 1.9 fold, 2 fold, 2.5 fold, 3 fold, 3.5 fold, 4 fold, 4.5 fold, or 5 fold.

In certain embodiments, an antibody described herein comprises the constant region of an IgG₂ antibody and the cysteine at amino acid residue 127 of the heavy chain, numbered according to the EU numbering system, is substituted for serine.

In certain embodiments, an antibody described herein comprises the constant region of an IgG₄ antibody and the serine at amino acid residue 228 of the heavy chain, numbered according to the EU numbering system, is substituted for proline.

In certain embodiments, any of the constant region mutations or modifications described herein can be introduced into one or both heavy chain constant regions of an antibody described herein having two heavy chain constant regions.

### Pharmaceutical Compositions

Provided herein are compositions comprising an anti-ILT2 antibody disclosed herein having the desired degree of purity in a physiologically acceptable carrier, excipient, or stabilizer (see, e.g., Remington's Pharmaceutical Sciences (1990) Mack Publishing Co., Easton, PA). Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates, including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In a specific embodiment, pharmaceutical compositions comprise an anti-ILT2 antibody disclosed herein, and optionally one or more additional prophylactic or therapeutic agents, in a pharmaceutically acceptable carrier. In a specific embodiment, pharmaceutical compositions comprise an anti-ILT2 antibody disclosed herein, and optionally one or more additional prophylactic or therapeutic agents, in a pharmaceutically acceptable carrier. In certain embodiments, the antibody is the only active ingredient included in the pharmaceutical composition. Pharmaceutical compositions described herein can be useful in decreasing or blocking ILT2 (e.g., human ILT2) activity and treating a condition, such as cancer. In certain embodiments, the present disclosure relates to a pharmaceutical composition of the present disclosure comprising an anti-ILT2 antibody of the present disclosure for use as a medicament. In another embodiment, the present disclosure relates to a pharmaceutical composition of the present disclosure for use in a method for the treatment of cancer.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering, or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcellulose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol, and propylene glycol for water miscible vehicles; and sodium hydroxide, hydrochloric acid, citric acid or lactic acid for pH adjustment.

A pharmaceutical composition can be formulated for any route of administration to a subject. Specific examples of routes of administration include intranasal, oral, pulmonary, transdermal, intradermal, and parenteral. Parenteral administration, characterized by either subcutaneous, intramuscular, or intravenous injection, is also contemplated herein. Injectables can be prepared in conventional forms, either as liquid solutions or suspensions, solid forms suitable for solution or suspension in liquid prior to injection, or as emulsions. The injectables, solutions, and emulsions also contain one or more excipients. Suitable excipients are, for example, water, saline, dextrose, glycerol, or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate, and cyclodextrins.

Preparations for parenteral administration of antibody include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol, and mixtures thereof.

Topical mixtures comprising an antibody are prepared as described for the local and systemic administration. The resulting mixture can be a solution, suspension, emulsions, or the like and can be formulated as creams, gels, ointments, emulsions, solutions, elixirs, lotions, suspensions, tinctures, pastes, foams, aerosols, irrigations, sprays, suppositories, bandages, dermal patches, or any other formulations suitable for topical administration.

An anti-ILT2 antibody disclosed herein can be formulated as an aerosol for topical application, such as by inhalation (see, e.g., U.S. Pat. Nos. 4,044,126, 4,414,209 and 4,364,923, which describe aerosols for delivery of a steroid useful for treatment of inflammatory diseases, particularly asthma and are herein incorporated by reference in their entireties). These formulations for administration to the respiratory tract can be in the form of an aerosol or solution for a nebulizer, or as a microfine powder for insufflations, alone or in combination with an inert carrier such as lactose. In such a case, the particles of the formulation will, in certain embodiments, have diameters of less than 50 microns, In certain embodiments less than 10 microns.

An anti-ILT2 antibody disclosed herein can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the antibody alone or in combination with other pharmaceutically acceptable excipients can also be administered.

Transdermal patches, including iontophoretic and electrophoretic devices, are well known to those of skill in the art, and can be used to administer an antibody. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957, all of which are herein incorporated by reference in their entireties.

In certain embodiments, a pharmaceutical composition comprising antibody described herein is a lyophilized powder, which can be reconstituted for administration as solutions, emulsions, and other mixtures. It may also be reconstituted and formulated as solids or gels. The lyophilized powder is prepared by dissolving antibody described herein, or a pharmaceutically acceptable derivative thereof, in a suitable solvent. In certain embodiments, the lyophilized powder is sterile. The solvent may contain an excipient which improves the stability or other pharmacological component of the powder or reconstituted solution, prepared from the powder. Excipients that may be used include, but are not limited to, dextrose, sorbitol, fructose, corn syrup, xylitol, glycerin, glucose, sucrose, or other suitable agent. The solvent may also contain a buffer, such as citrate, sodium or potassium phosphate or other such buffer known to those of skill in the art at, In certain embodiments, about neutral pH. Subsequent sterile filtration of the solution followed by lyophilization under standard conditions known to those of skill in the art provides the desired formulation. In certain embodiments, the resulting solution will be apportioned into vials for lyophilization. Each vial will contain a single dosage or multiple dosages of the compound. The lyophilized powder can be stored under appropriate conditions, such as at about 4° C. to room temperature. Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

The anti-ILT2 antibodies disclosed herein and other compositions provided herein can also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874, all of which are herein incorporated by reference in their entireties. In a specific embodiment, an antibody described herein is targeted to a tumor.

The compositions to be used for in vivo administration can be sterile. This is readily accomplished by filtration through, e.g., sterile filtration membranes.

### Methods of Use and Uses

In another aspect, the instant disclosure provides a method of treating a subject using the anti-ILT2 antibodies disclosed herein. Any disease or disorder in a subject that would benefit from decrease of ILT2 (e.g., human ILT2) function can be treated using the anti-ILT2 antibodies disclosed herein. In certain embodiments, the disease or disorder is resistant to a checkpoint targeting agent (e.g., an antagonist anti-CTLA-4 antibody, an antagonist anti-PD-L1 antibody, an antagonist anti-PD-L2 antibody, or an antagonist anti-PD-1 antibody). In certain embodiments, the disease or disorder is recurrent after treatment with a checkpoint targeting agent (e.g., an antagonist anti-CTLA-4 antibody, an antagonist anti-PD-L1 antibody, an antagonist anti-PD-L2 antibody, or an antagonist anti-PD-1 antibody).

The anti-ILT2 antibodies disclosed herein are particularly useful for inhibiting immune system tolerance to tumors, and accordingly can be used as an immunotherapy for subjects with cancer. For example, in certain embodiments, the instant disclosure provides a method of increasing T cell (e.g., CD8⁺ cytotoxic T cells, CD4⁺ helper T cells, NKT cells, effector T cells, or memory T cells) activation in response to an antigen in a subject, the method comprising administering to the subject an effective amount of an anti-ILT2 antibody or pharmaceutical composition thereof as disclosed herein. In certain embodiments, the instant disclosure provides a method of treating cancer in a subject, the method comprising administering to the subject an effective amount of the antibody or pharmaceutical composition, as disclosed herein.

Cancers that can be treated with the anti-ILT2 antibodies or pharmaceutical compositions disclosed herein include, without limitation, a solid tumor, a hematological cancer (e.g., leukemia, lymphoma, myeloma, e.g., multiple myeloma), and a metastatic lesion. In certain embodiments, the cancer is a solid tumor. Examples of solid tumors include malignancies, e.g., sarcomas and carcinomas, e.g., adenocarcinomas of the various organ systems, such as those affecting the lung, breast, ovarian, lymphoid, gastrointestinal (e.g., colon), anal, genitals and genitourinary tract (e.g., renal, urothelial, bladder cells, prostate), pharynx, CNS (e.g., brain, neural or glial cells), head and neck, skin (e.g., melanoma), and pancreas, as well as adenocarcinomas which include malignancies such as colon cancers, rectal cancer, renal-cell carcinoma, liver cancer, lung cancer (e.g., non-small cell lung cancer or small cell lung cancer), cancer of the small intestine and cancer of the esophagus. The cancer may be at an early, intermediate, late stage, or metastatic cancer. In certain embodiments, the cancer is resistant to a checkpoint targeting agent (e.g., an antagonist anti-CTLA-4 antibody, an antagonist anti-PD-L1 antibody, an antagonist anti-PD-L2 antibody, or an antagonist anti-PD-1 antibody). In certain embodiments, the cancer is recurrent after treatment with a checkpoint targeting agent (e.g., an antagonist anti-CTLA-4 antibody, an antagonist anti-PD-L1 antibody, an antagonist anti-PD-L2 antibody, or an antagonist anti-PD-1 antibody).

In certain embodiments, the cancer is chosen from lung cancer (e.g., lung adenocarcinoma or non-small cell lung cancer (NSCLC) (e.g., NSCLC with squamous and/or non-squamous histology, or NSCLC adenocarcinoma)), melanoma (e.g., an advanced melanoma), renal cancer (e.g., a renal cell carcinoma), liver cancer (e.g., hepatocellular carcinoma), myeloma (e.g., a multiple myeloma), a prostate cancer, a breast cancer (e.g., a breast cancer that does not express one, two or all of estrogen receptor, progesterone receptor, or Her2/neu, e.g., a triple negative breast cancer), an ovarian cancer, a colorectal cancer, a pancreatic cancer, a head and neck cancer (e.g., head and neck squamous cell carcinoma (HNSCC)), anal cancer, gastro-esophageal cancer (e.g., esophageal squamous cell carcinoma), mesothelioma, nasopharyngeal cancer, thyroid cancer, cervical cancer, epithelial cancer, peritoneal cancer, or a lymphoproliferative disease (e.g., a post-transplant lymphoproliferative disease).

In certain embodiments, the cancer is a hematological cancer, for example, a leukemia, a lymphoma, or a myeloma. In certain embodiments, the cancer is a leukemia, for example, acute lymphoblastic leukemia (ALL), acute myelogenous leukemia (AML), acute myeloblastic leukemia (AML), chronic lymphocytic leukemia (CLL), chronic myelogenous leukemia (CML), chronic myeloid leukemia (CML), chronic myelomonocytic leukemia (CMML), chronic lymphocytic leukemia (CLL), or hairy cell leukemia. In certain embodiments, the cancer is a lymphoma, for example, B cell lymphoma, diffuse large B-cell lymphoma (DLBCL), activated B-cell like (ABC) diffuse large B cell lymphoma, germinal center B cell (GCB) diffuse large B cell lymphoma, mantle cell lymphoma, Hodgkin lymphoma, non-Hodgkin lymphoma, relapsed non-Hodgkin lymphoma, refractory non-Hodgkin lymphoma, recurrent follicular non-Hodgkin lymphoma, Burkitt lymphoma, small lymphocytic lymphoma, follicular lymphoma, lymphoplasmacytic lymphoma, or extranodal marginal zone lymphoma. In certain embodiments the cancer is a myeloma, for example, multiple myeloma.

In another embodiment, the cancer is chosen from a carcinoma (e.g., advanced or metastatic carcinoma), melanoma or a lung carcinoma, e.g., a non-small cell lung carcinoma.

In certain embodiments, the cancer is a lung cancer, e.g., a lung adenocarcinoma, non-small cell lung cancer, or small cell lung cancer.

In certain embodiments, the cancer is a melanoma, e.g., an advanced melanoma. In certain embodiments, the cancer is an advanced or unresectable melanoma that does not respond to other therapies. In other embodiments, the cancer is a melanoma with a BRAF mutation (e.g., a BRAF V600 mutation). In yet other embodiments, the anti-ILT2 antibodies or pharmaceutical composition disclosed herein is administered after treatment with an anti-CTLA-4 antibody (e.g., ipilimumab) with or without a BRAF inhibitor (e.g., vemurafenib or dabrafenib).

In another embodiment, the cancer is a hepatocarcinoma, e.g., an advanced hepatocarcinoma, with or without a viral infection, e.g., a chronic viral hepatitis.

In another embodiment, the cancer is a prostate cancer, e.g., an advanced prostate cancer.

In yet another embodiment, the cancer is a myeloma, e.g., multiple myeloma.

In yet another embodiment, the cancer is a renal cancer, e.g., a renal cell carcinoma (RCC) (e.g., a metastatic RCC, clear cell renal cell carcinoma (CCRCC) or kidney papillary cell carcinoma).

In yet another embodiment, the cancer is chosen from a lung cancer, a melanoma, a renal cancer, a breast cancer, a colorectal cancer, a leukemia, or a metastatic lesion of the cancer.

In certain embodiments, these methods further comprise administering an additional therapeutic agent to the subject. In certain embodiments, the additional therapeutic agent is a chemotherapeutic, a radiotherapeutic, or a checkpoint targeting agent. In certain embodiments, the chemotherapeutic agent is a hypomethylating agent (e.g., azacitidine). In certain embodiments, the chemotherapeutic agent is a DNA damage-inducing agent (e.g., gemcitabine). In certain embodiments, the checkpoint targeting agent is selected from the group consisting of an antagonist anti-CTLA-4 antibody, an antagonist anti-PD-L1 antibody, an antagonist anti-PD-L2 antibody, an antagonist anti-PD-1 antibody, an antagonist anti-TIM-3 antibody, an antagonist anti-LAG-3 antibody, an antagonist anti-VISTA antibody, an antagonist anti-CD96 antibody, an antagonist anti-CEACAM1 antibody, an agonist anti-CD137 antibody, an agonist anti-GITR antibody, and an agonist anti-OX40 antibody. In certain embodiments, the checkpoint targeting agent is selected from the group consisting of an antagonist anti-CTLA-4 antibody, an antagonist anti-PD-L1 antibody, an antagonist anti-PD-L2 antibody, and an antagonist anti-PD-1 antibody, wherein the ILT2 (e.g., human ILT2) antibodies or pharmaceutical compositions disclosed herein synergize with the checkpoint targeting agent.

In certain embodiments, the present disclosure relates to an antibody and/or pharmaceutical composition of the present disclosure for use in a method of the present disclosure, wherein the method further comprises administering an additional therapeutic agent to the subject. In certain embodiments, the present disclosure relates to (a) an antibody and/or pharmaceutical composition of the present disclosure and (b) an additional therapeutic agent for use as a medicament. In certain embodiments, the present disclosure relates to (a) an antibody and/or pharmaceutical composition of the present disclosure and (b) an additional therapeutic agent for use in a method for the treatment of cancer. In a further embodiment, the present disclosure relates to a pharmaceutical composition, kit or kit-of-parts comprising (a) an antibody and/or pharmaceutical composition of the present disclosure and (b) an additional therapeutic agent. In certain embodiments, the additional therapeutic agent is a chemotherapeutic, a radiotherapeutic, or a checkpoint targeting agent.

In certain embodiments, an anti-PD-1 antibody is used in methods disclosed herein. In certain embodiments, the anti-PD-1 antibody is nivolumab, also known as BMS-936558 or MDX1106, developed by Bristol-Myers Squibb. In certain embodiments, the anti-PD-1 antibody is pembrolizumab, also known as lambrolizumab or MK-3475, developed by Merck & Co. In certain embodiments, the anti-PD-1 antibody is pidilizumab, also known as CT-011, developed by CureTech. In certain embodiments, the anti-PD-1 antibody is MEDI0680, also known as AMP-514, developed by Medimmune. In certain embodiments, the anti-PD-1 antibody is PDR001 developed by Novartis Pharmaceuticals. In certain embodiments, the anti-PD-1 antibody is REGN2810 developed by Regeneron Pharmaceuticals. In certain embodiments, the anti-PD-1 antibody is PF-06801591 developed by Pfizer. In certain embodiments, the anti-PD-1 antibody is BGB-A317 developed by BeiGene. In certain embodiments, the anti-PD-1 antibody is TSR-042 developed by AnaptysBio and Tesaro. In certain embodiments, the anti-PD-1 antibody is SHR-1210 developed by Hengrui.

Further non-limiting examples of anti-PD-1 antibodies that may be used in treatment methods disclosed herein are disclosed in the following patents and patent applications, all of which are herein incorporated by reference in their entireties for all purposes: U.S. Pat. Nos. 6,808,710; 7,332,582; 7,488,802; 8,008,449; 8,114,845; 8,168,757; 8,354,509; 8,686,119; 8,735,553; 8,747,847; 8,779,105; 8,927,697; 8,993,731; 9,102,727; 9,205,148; U.S. Publication No. US 2013/0202623 A1; U.S. Publication No. US 2013/0291136 A1; U.S. Publication No. US 2014/0044738 A1; U.S. Publication No. US 2014/0356363 A1; U.S. Publication No. US 2016/0075783 A1; and PCT Publication No. WO 2013/033091 A1; PCT Publication No. WO 2015/036394 A1; PCT Publication No. WO 2014/179664 A2; PCT Publication No. WO 2014/209804 A1; PCT Publication No. WO 2014/206107 A1; PCT Publication No. WO 2015/058573 A1; PCT Publication No. WO 2015/085847 A1; PCT Publication No. WO 2015/200119 A1; PCT Publication No. WO 2016/015685 A1; and PCT Publication No. WO 2016/020856 A1.

In certain embodiments, an anti-PD-L1 antibody is used in methods disclosed herein. In certain embodiments, the anti-PD-L1 antibody is atezolizumab developed by Genentech. In certain embodiments, the anti-PD-L1 antibody is durvalumab developed by AstraZeneca, Celgene, and Medimmune. In certain embodiments, the anti-PD-L1 antibody is avelumab, also known as MSB0010718C, developed by Merck Serono and Pfizer. In certain embodiments, the anti-PD-L1 antibody is MDX-1105 developed by Bristol-Myers Squibb. In certain embodiments, the anti-PD-L1 antibody is AMP-224 developed by Amplimmune and GSK.

Non-limiting examples of anti-PD-L1 antibodies that may be used in treatment methods disclosed herein are disclosed in the following patents and patent applications, all of which are herein incorporated by reference in their entireties for all purposes: U.S. Pat. Nos. 7,943,743; 8,168,179; 8,217,149; 8,552,154; 8,779,108; 8,981,063; 9,175,082; U.S. Publication No. US 2010/0203056 A1; U.S. Publication No. US 2003/0232323 A1; U.S. Publication No. US 2013/0323249 A1; U.S. Publication No. US 2014/0341917 A1; U.S. Publication No. US 2014/0044738 A1; U.S. Publication No. US 2015/0203580 A1; U.S. Publication No. US 2015/0225483 A1; U.S. Publication No. US 2015/0346208 A1; U.S. Publication No. US 2015/0355184 A1; and PCT Publication No. WO 2014/100079 A1; PCT Publication No. WO 2014/022758 A1; PCT Publication No. WO 2014/055897 A2; PCT Publication No. WO 2015/061668 A1; PCT Publication No. WO 2015/109124 A1; PCT Publication No. WO 2015/195163 A1; PCT Publication No. WO 2016/000619 A1; and PCT Publication No. WO 2016/030350 A1.

In certain embodiments, an anti-CTLA-4 antibody is used in methods disclosed herein. In certain embodiments, the anti-CTLA-4 antibody is ipilimumab developed by Bristol-Myers Squibb.

In certain embodiments, an anti-ILT2 antibody disclosed herein is administered to a subject in combination with a compound that targets an immunomodulatory enzyme(s) such as IDO (indoleamine-(2,3)-dioxygenase) and/or TDO (tryptophan 2,3-dioxygenase). Therefore, in certain embodiments, the additional therapeutic agent is a compound that targets an immunomodulatory enzyme(s), such as an inhibitor of indoleamine-(2,3)-dioxygenase (IDO). In certain embodiments, such compound is selected from the group consisting of epacadostat (Incyte Corp; see, e.g., WO 2010/005958 which is herein incorporated by reference in its entirety), F001287 (Flexus Biosciences/Bristol-Myers Squibb), indoximod (NewLink Genetics), and NLG919 (NewLink Genetics). In certain embodiments, the compound is epacadostat. In another embodiment, the compound is F001287. In another embodiment, the compound is indoximod. In another embodiment, the compound is NLG919. In a specific embodiment, an anti-ILT2 antibody disclosed herein is administered to a subject in combination with an IDO inhibitor for treating cancer. The IDO inhibitor as described herein for use in treating cancer is present in a solid dosage form of a pharmaceutical composition such as a tablet, a pill, or a capsule, wherein the pharmaceutical composition includes an IDO inhibitor and a pharmaceutically acceptable excipient. As such, the antibody as described herein and the IDO inhibitor as described herein can be administered separately, sequentially, or concurrently as separate dosage forms. In certain embodiments, the antibody is administered parenterally, and the IDO inhibitor is administered orally. In certain embodiments, the inhibitor is selected from the group consisting of epacadostat (Incyte Corporation), F001287 (Flexus Biosciences/Bristol-Myers Squibb), indoximod (NewLink Genetics), and NLG919 (NewLink Genetics). Epacadostat has been described in PCT Publication No. WO 2010/005958, which is herein incorporated by reference in its entirety for all purposes. In certain embodiments, the inhibitor is epacadostat. In another embodiment, the inhibitor is F001287. In another embodiment, the inhibitor is indoximod. In another embodiment, the inhibitor is NLG919.

In certain embodiments, an anti-ILT2 antibody disclosed herein is administered to a subject in combination with a vaccine. The vaccine can be, e.g., a peptide vaccine, a DNA vaccine, or an RNA vaccine.

In certain embodiments, an anti-ILT2 antibody disclosed herein is administered to a subject in combination with an adjuvant. Various adjuvants can be used depending on the treatment context. Non-limiting examples of appropriate adjuvants include, but not limited to, Complete Freund's Adjuvant (CFA), Incomplete Freund's Adjuvant (IFA), montanide ISA (incomplete Seppic adjuvant), the Ribi adjuvant system (RAS), Titer Max, muramyl peptides, Syntex Adjuvant Formulation (SAF), alum (aluminum hydroxide and/or aluminum phosphate), aluminum salt adjuvants, Gerbu® adjuvants, nitrocellulose absorbed antigen, encapsulated or entrapped antigen, 3 De-O-acylated monophosphoryl lipid A (3 D-MPL), immunostimulatory oligonucleotides, toll-like receptor (TLR) ligands, mannan-binding lectin (MBL) ligands, STING agonists, immuno-stimulating complexes such as saponins, Quil A, QS-21, QS-7, ISCOMATRIX, and others. Other adjuvants include CpG oligonucleotides and double stranded RNA molecules, such as poly(A) and poly(U). Combinations of the above adjuvants may also be used. See, e.g., U.S. Pat. Nos. 6,645,495; 7,029,678; and 7,858,589, all of which are incorporated herein by reference in their entireties. In certain embodiments, the adjuvant used herein is QS-21 STIMULON.

In certain embodiments, an anti-ILT2 antibody disclosed herein is administered to a subject in combination with an additional therapeutic agent comprising a TCR. In certain embodiments, the additional therapeutic agent is a soluble TCR. In certain embodiments, the additional therapeutic agent is a cell expressing a TCR. Therefore, in certain embodiments, the present disclosure relates to an antibody and/or pharmaceutical composition of the present disclosure in combination with an additional therapeutic agent comprising a TCR for use as a medicament and/or for use in a method for the treatment of cancer.

In certain embodiments, an anti-ILT2 antibody disclosed herein is administered to a subject in combination with a cell expressing a chimeric antigen receptor (CAR). In certain embodiments, the cell is a T cell.

In certain embodiments, an anti-ILT2 antibody disclosed herein is administered to a subject in combination with a TCR mimic antibody. In certain embodiments, the TCR mimic antibody is an antibody that specifically binds to a peptide-MHC complex. For non-limiting examples of TCR mimic antibodies, see, e.g., U.S. Pat. No. 9,074,000 and U.S. Publication Nos. US 2009/0304679 A1 and US 2014/0134191 A1, all of which are incorporated herein by reference in their entireties.

In certain embodiments, an anti-ILT2 antibody disclosed herein is administered to a subject in combination with a bispecific T-cell engager (BiTE) (e.g., as described in WO2005061547A2, which is incorporated by reference herein in its entirety) and/or a dual-affinity re-targeting antibody (DART) (e.g., as described in WO2012162067A2, which is incorporated by reference herein in its entirety). In certain embodiments, the BiTE and/or DART specifically binds to a tumor-associated antigen (e.g., a polypeptide overexpressed in a tumor, a polypeptide derived from an oncovirus, a polypeptide comprising a post-translational modification specific to a tumor, a polypeptide specifically mutated in a tumor) and a molecule on an effector cell (e.g., CD3 or CD16). In certain embodiments, the tumor-associated antigen is EGFR (e.g., human EGFR), optionally wherein the BiTE and/or DART comprises the VH and VL sequences of cetuximab. In certain embodiments, the tumor-associated antigen is Her2 (e.g., human Her2), optionally wherein the BiTE and/or DART comprises the VH and VL sequences of trastuzumab. In certain embodiments, the tumor-associated antigen is CD20 (e.g., human CD20).

The anti-ILT2 antibody and the additional therapeutic agent (e.g., chemotherapeutic, radiotherapeutic, checkpoint targeting agent, IDO inhibitor, vaccine, adjuvant, a soluble TCR, a cell expressing a TCR, a cell expressing a chimeric antigen receptor, and/or a TCR mimic antibody) can be administered separately, sequentially, or concurrently as separate dosage forms. In certain embodiments, an anti-ILT2 antibody is administered parenterally, and an IDO inhibitor is administered orally.

An antibody or pharmaceutical composition described herein may be delivered to a subject by a variety of routes. These include, but are not limited to, parenteral, intranasal, intratracheal, oral, intradermal, topical, intramuscular, intraperitoneal, transdermal, intravenous, intratumoral, conjunctival, intra-arterial, and subcutaneous routes. Pulmonary administration can also be employed, e.g., by use of an inhaler or nebulizer, and formulation with an aerosolizing agent for use as a spray. In certain embodiments, the antibody or pharmaceutical composition described herein is delivered subcutaneously or intravenously. In certain embodiments, the antibody or pharmaceutical composition described herein is delivered intra-arterially. In certain embodiments, the antibody or pharmaceutical composition described herein is delivered intratumorally. In certain embodiments, the antibody or pharmaceutical composition described herein is delivered into a tumor draining lymph node.

The amount of an antibody or composition which will be effective in the treatment and/or prevention of a condition will depend on the nature of the disease, and can be determined by standard clinical techniques.

The precise dose to be employed in a composition will also depend on the route of administration, and the seriousness of the infection or disease caused by it, and should be decided according to the judgment of the practitioner and each subject's circumstances. For example, effective doses may also vary depending upon means of administration, target site, physiological state of the patient (including age, body weight, and health), whether the patient is human or an animal, other medications administered, or whether treatment is prophylactic or therapeutic. Usually, the patient is a human, but non-human mammals, including transgenic mammals, can also be treated. Treatment dosages are optimally titrated to optimize safety and efficacy.

An anti-ILT2 antibody described herein can also be used to assay ILT2 (e.g., human ILT2) protein levels in a biological sample using classical immunohistological methods known to those of skill in the art, including immunoassays, such as the enzyme linked immunosorbent assay (ELISA), immunoprecipitation, or Western blotting. Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin. Such labels can be used to label an antibody described herein. Alternatively, a second antibody that recognizes an anti-ILT2 antibody described herein can be labeled and used in combination with an anti-ILT2 antibody to detect ILT2 (e.g., human ILT2) protein levels. Therefore, in certain embodiments, the present disclosure relates to the use of an anti-ILT2 antibody of the present disclosure for in vitro detection of ILT2 (e.g., human ILT2) protein in a biological sample. In a further embodiment, the present disclosure relates to the use of an anti-ILT2 antibody of the disclosure, for assaying and/or detecting ILT2 (e.g., human ILT2) protein levels in a biological sample in vitro, optionally wherein the anti-ILT2 antibody is conjugated to a radionuclide or detectable label, and/or carries a label described herein, and/or wherein an immunohistological method is used.

Assaying for the expression level of ILT2 (e.g., human ILT2) protein is intended to include qualitatively or quantitatively measuring or estimating the level of ILT2 (e.g., human ILT2) protein in a first biological sample either directly (e.g., by determining or estimating absolute protein level) or relatively (e.g., by comparing to the disease associated protein level in a second biological sample). ILT2 (e.g., human ILT2) polypeptide expression level in the first biological sample can be measured or estimated and compared to a standard ILT2 (e.g., human ILT2) protein level, the standard being taken, for example, from a second biological sample obtained from an individual not having the disorder or being determined by averaging levels from a population of individuals not having the disorder. As will be appreciated in the art, once the “standard” ILT2 (e.g., human ILT2) polypeptide level is known, it can be used repeatedly as a standard for comparison. Therefore, in a further embodiment, the present disclosure relates to an in vitro method for assaying and/or detecting ILT2 protein levels, for example human ILT2 protein levels, in a biological sample, comprising qualitatively or quantitatively measuring or estimating the level of ILT2 protein, for example of human ILT2 protein, in a biological sample, by an immunohistological method.

As used herein, the term “biological sample” refers to any biological sample obtained from a subject, cell line, tissue, or other source of cells potentially expressing ILT2 (e.g., human ILT2). Methods for obtaining tissue biopsies and body fluids from animals (e.g., humans or cynomolgus monkeys) are well known in the art. Biological samples include peripheral blood mononuclear cells (PBMCs).

An anti-ILT2 antibody described herein can be used for prognostic, diagnostic, monitoring, and screening applications, including in vitro and in vivo applications well known and standard to the skilled artisan and based on the present description. Prognostic, diagnostic, monitoring, and screening assays and kits for in vitro assessment and evaluation of immune system status and/or immune response may be utilized to predict, diagnose, and monitor to evaluate patient samples, including those known to have or suspected of having an immune system-dysfunction or with regard to an anticipated or desired immune system response, antigen response, or vaccine response. The assessment and evaluation of immune system status and/or immune response are also useful in determining the suitability of a patient for a clinical trial of a drug or for the administration of a particular chemotherapeutic agent, a radiotherapeutic agent, or an antibody, including combinations thereof, versus a different agent or antibody. This type of prognostic and diagnostic monitoring and assessment is already in practice utilizing antibodies against the HER2 protein in breast cancer (HercepTest™, Dako) where the assay is also used to evaluate patients for antibody therapy using Herceptin®. In vivo applications include directed cell therapy and immune system modulation and radio imaging of immune responses. Therefore, in certain embodiments, the present disclosure relates to an anti-ILT2 antibody and/or pharmaceutical composition of the present disclosure for use as a diagnostic. In certain embodiments, the present disclosure relates to an anti-ILT2 antibody and/or pharmaceutical composition of the present disclosure for use in a method for the prediction, diagnosis and/or monitoring of a subject having or suspected to have an immune system-dysfunction and/or with regard to an anticipated or desired immune system response, antigen response or vaccine response. In another embodiment, the present disclosure relates to the use of an anti-ILT2 antibody of the disclosure, for predicting, diagnosing and/or monitoring of a subject having or suspected to have an immune system-dysfunction and/or with regard to an anticipated or desired immune system response, antigen response or vaccine response by assaying and/or detecting human ILT2 protein levels in a biological sample of the subject in vitro.

In certain embodiments, an anti-ILT2 antibody can be used in immunohistochemistry of biopsy samples. In certain embodiments, the method is an in vitro method. In another embodiment, an anti-ILT2 antibody can be used to detect levels of ILT2 (e.g., human ILT2), or levels of cells which contain ILT2 (e.g., human ILT2) on their membrane surface, the levels of which can then be linked to certain disease symptoms. Anti-ILT2 antibodies described herein may carry a detectable or functional label and/or may be conjugated to a radionuclide or detectable label. When fluorescence labels are used, currently available microscopy and fluorescence-activated cell sorter analysis (FACS) or combination of both methods procedures known in the art may be utilized to identify and to quantitate the specific binding members. Anti-ILT2 antibodies described herein may carry or may be conjugated to a fluorescence label. Exemplary fluorescence labels include, for example, reactive and conjugated probes, e.g., Aminocoumarin, Fluorescein and Texas red, Alexa Fluor dyes, Cy dyes and DyLight dyes. An anti-ILT2 antibody may carry or may be conjugated to a radioactive label or radionuclide, such as the isotopes ³H, ¹⁴C, ³²P, ³⁵S, ³⁶Cl, ⁵¹Cr, ⁵⁷Co, ⁵⁸Co, ⁵⁹Fe, ⁶⁷Cu, ⁹⁰Y, ⁹⁹Tc, ¹¹¹In, ¹¹⁷Lu, ¹²¹I, ¹²⁴I, ¹²⁵I, ¹³¹I, ¹⁹⁸Au, ²¹¹At, ²¹³Bi, ²²⁵Ac and, ¹⁸⁶Re. When radioactive labels are used, currently available counting procedures known in the art may be utilized to identify and quantitate the specific binding of an anti-ILT2 antibody to ILT2 (e.g., human ILT2). In the instance where the label is an enzyme, detection may be accomplished by any of the presently utilized colorimetric, spectrophotometric, fluorospectrophotometric, amperometric, or gasometric techniques as known in the art. This can be achieved by contacting a sample or a control sample with an anti-ILT2 antibody under conditions that allow for the formation of a complex between the anti-ILT2 antibody and ILT2 (e.g., human ILT2). Any complexes formed between the anti-ILT2 antibody and ILT2 (e.g., human ILT2), are detected and compared in the sample and the control. In light of the specific binding of the anti-ILT2 antibodies described herein for ILT2 (e.g., human ILT2), the anti-ILT2 antibodies can be used to specifically detect ILT2 (e.g., human ILT2). The anti-ILT2 antibodies described herein can also be used to purify ILT2 (e.g., human ILT2) via immunoaffinity purification. Also included herein is an assay system which may be prepared in the form of a test kit, kit, or kit-of-parts for the quantitative analysis of the extent of the presence of, for instance, ILT2 (e.g., human ILT2)/ILT2 (e.g., human ILT2) ligand complexes. The system, test kit, kit, or kit-of-parts may comprise a labeled component, e.g., a labeled antibody, and one or more additional immunochemical reagents.

### Polynucleotides, Vectors, and Methods of Producing Antibodies

In another aspect, provided herein are polynucleotides comprising a nucleotide sequence encoding an antibody, or a portion thereof, described herein or a fragment thereof (e.g., a VL and/or VH; and a light chain and/or heavy chain) that specifically binds to an ILT2 (e.g., human ILT2) antigen, and vectors, e.g., vectors comprising such polynucleotides for recombinant expression in host cells (e.g., E. coli and mammalian cells). Provided herein are polynucleotides comprising nucleotide sequences encoding a heavy and/or light chain of any of the antibodies provided herein, as well as vectors comprising such polynucleotide sequences, e.g., expression vectors for their efficient expression in host cells, e.g., mammalian cells.

As used herein, an “isolated” polynucleotide or nucleic acid molecule is one which is separated from other nucleic acid molecules which are present in the natural source (e.g., in a mouse or a human) of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. For example, the language “substantially free” includes preparations of polynucleotide or nucleic acid molecules having less than about 15%, 10%, 5%, 2%, 1%, 0.5%, or 0.1% (in particular less than about 10%) of other material, e.g., cellular material, culture medium, other nucleic acid molecules, chemical precursors, and/or other chemicals. In a specific embodiment, a nucleic acid molecule(s) encoding an antibody described herein is isolated or purified.

In particular aspects, provided herein are polynucleotides comprising nucleotide sequences encoding antibodies, which specifically bind to an ILT2 (e.g., human ILT2) polypeptide and comprises an amino acid sequence as described herein, as well as antibodies which compete with such antibodies for binding to an ILT2 (e.g., human ILT2) polypeptide (e.g., in a dose-dependent manner), or which binds to the same epitope as that of such antibodies.

In certain aspects, provided herein are polynucleotides comprising a nucleotide sequence encoding the light chain or heavy chain of antibody described herein. The polynucleotides can comprise nucleotide sequences encoding a light chain comprising the VL FRs and CDRs of antibodies described herein (see, e.g., Table 2) or nucleotide sequences encoding a heavy chain comprising the VH FRs and CDRs of antibodies described herein (see, e.g., Table 2). In certain embodiments, a polynucleotide encodes a VH, VL, heavy chain, and/or light chain of a described herein. In another embodiment, a polynucleotide encodes the first VH and the first VL of a described herein. In another embodiment, a polynucleotide encodes the second VH and the second VL of a described herein. In another embodiment, a polynucleotide encodes the first heavy chain and the first light chain of a described herein. In another embodiment, a polynucleotide encodes the second heavy chain and the second light chain of a described herein. In another embodiment, a polynucleotide encodes the VH and/or the VL, or the heavy chain and/or the light chain, of an antibody described herein.

Also provided herein are polynucleotides encoding an anti-ILT2 antibody that are optimized, e.g., by codon/RNA optimization, replacement with heterologous signal sequences, and elimination of mRNA instability elements. Methods to generate optimized nucleic acids encoding an anti-ILT2 antibody or a fragment thereof (e.g., light chain, heavy chain, VH domain, or VL domain) for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly, all of which are herein incorporated by reference in their entireties. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In certain embodiments, it can be desirable to alter one or more codons to encode a conservative mutation, e.g., a similar amino acid with similar chemical structure and properties and/or function as the original amino acid. Such methods can increase expression of an anti-ILT2 antibody or fragment thereof by at least 1 fold, 2 fold, 3 fold, 4 fold, 5 fold, 10 fold, 20 fold, 30 fold, 40 fold, 50 fold, 60 fold, 70 fold, 80 fold, 90 fold, or 100 fold or more relative to the expression of an anti-ILT2 antibody encoded by polynucleotides that have not been optimized.

In certain embodiments, an optimized polynucleotide sequence encoding an anti-ILT2 antibody described herein or a fragment thereof (e.g., VL domain and/or VH domain) can hybridize to an antisense (e.g., complementary) polynucleotide of an unoptimized polynucleotide sequence encoding an anti-ILT2 antibody described herein or a fragment thereof (e.g., VL domain and/or VH domain). In specific embodiments, an optimized nucleotide sequence encoding an anti-ILT2 antibody described herein or a fragment hybridizes under high stringency conditions to antisense polynucleotide of an unoptimized polynucleotide sequence encoding an anti-ILT2 antibody described herein or a fragment thereof. In a specific embodiment, an optimized nucleotide sequence encoding an anti-ILT2 antibody described herein or a fragment thereof hybridizes under high stringency, intermediate or lower stringency hybridization conditions to an antisense polynucleotide of an unoptimized nucleotide sequence encoding an anti-ILT2 antibody described herein or a fragment thereof. Information regarding hybridization conditions has been described, see, e.g., U.S. Patent Application Publication No. US 2005/0048549 (e.g., paragraphs 72-73), which is herein incorporated by reference in its entirety.

The polynucleotides can be obtained, and the nucleotide sequence of the polynucleotides determined, by any method known in the art. Nucleotide sequences encoding antibodies described herein, e.g., antibodies described in Table 2, and modified versions of these antibodies can be determined using methods well known in the art, i.e., nucleotide codons known to encode particular amino acids are assembled in such a way to generate a nucleic acid that encodes the antibody. Such a polynucleotide encoding the antibody can be assembled from chemically synthesized oligonucleotides (e.g., as described in Kutmeier G et al., (1994), BioTechniques 17:242-6, herein incorporated by reference in its entirety), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antigen-binding region of a described here or an antibody described herein can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising the sequence encoding the light chain and/or heavy chain of an antibody. Such PCR amplification methods can be used to obtain nucleic acids comprising the sequence encoding the variable light chain region and/or the variable heavy chain region of an antibody. The amplified nucleic acids can be cloned into vectors for expression in host cells and for further cloning.

If a clone containing a nucleic acid encoding a particular antigen-binding region or antibody is not available, but the sequence of the antigen-binding region or antibody molecule is known, a nucleic acid encoding the immunoglobulin can be chemically synthesized or obtained from a suitable source (e.g., an antibody cDNA library or a cDNA library generated from, or nucleic acid, preferably poly A+ RNA, isolated from, any tissue or cells expressing the antibody, such as hybridoma cells selected to express an antibody described herein) by PCR amplification using synthetic primers hybridizable to the 3′ and 5′ ends of the sequence, or by cloning using an oligonucleotide probe specific for the particular gene sequence to identify, e.g., a cDNA clone from a cDNA library that encodes the antibody. Amplified nucleic acids generated by PCR can then be cloned into replicable cloning vectors using any method well known in the art.

DNA encoding anti-ILT2 (e.g., human ILT2) antibodies described herein can be readily isolated and sequenced using conventional procedures (e.g., by using oligonucleotide probes that are capable of binding specifically to genes encoding the heavy and light chains of the anti-ILT2 (e.g., human ILT2) antibodies). Hybridoma cells can serve as a source of such DNA. Once isolated, the DNA can be placed into expression vectors, which are then transfected into host cells such as E. coli cells, simian COS cells, Chinese hamster ovary (CHO) cells (e.g., CHO cells from the CHO GS System™ (Lonza)), or myeloma cells that do not otherwise produce immunoglobulin protein, to obtain the synthesis of anti-ILT2 antibodies in the recombinant host cells.

To generate whole antibodies or antigen-binding regions, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a heavy chain constant region, e.g., the human gamma 1 or human gamma 4 constant region, and the PCR amplified VL domains can be cloned into vectors expressing a light chain constant region, e.g., human kappa or lambda constant regions. In certain embodiments, the vectors for expressing the VH or VL domains comprise an EF-1α promoter, a secretion signal, a cloning site for the variable region, constant regions, and a selection marker such as neomycin. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

The DNA also can be modified, for example, by substituting the coding sequence for human heavy and light chain constant regions in place of the murine sequences, or by covalently joining to the immunoglobulin coding sequence all or part of the coding sequence for a non-immunoglobulin polypeptide.

Also provided are polynucleotides that hybridize under high stringency, intermediate or lower stringency hybridization conditions to polynucleotides that encode an antibody described herein. In specific embodiments, polynucleotides described herein hybridize under high stringency, intermediate or lower stringency hybridization conditions to polynucleotides encoding a VH domain and/or VL domain provided herein.

Hybridization conditions have been described in the art and are known to one of skill in the art. For example, hybridization under stringent conditions can involve hybridization to filter-bound DNA in 6× sodium chloride/sodium citrate (SSC) at about 45° C. followed by one or more washes in 0.2×SSC/0.1% SDS at about 50-65° C.; hybridization under highly stringent conditions can involve hybridization to filter-bound nucleic acid in 6×SSC at about 45° C. followed by one or more washes in 0.1×SSC/0.2% SDS at about 68° C. Hybridization under other stringent hybridization conditions are known to those of skill in the art and have been described, see, for example, Ausubel F M et al., eds., (1989) Current Protocols in Molecular Biology, Vol. I, Green Publishing Associates, Inc. and John Wiley & Sons, Inc., New York at pages 6.3.1-6.3.6 and 2.10.3, which is herein incorporated by reference in its entirety.

In certain aspects, provided herein are cells (e.g., host cells) expressing (e.g., recombinantly) antibodies described herein which specifically bind to ILT2 (e.g., human ILT2), and related polynucleotides and expression vectors. Provided herein are vectors (e.g., expression vectors) comprising polynucleotides comprising nucleotide sequences encoding anti-ILT2 antibodies or a fragment for recombinant expression in host cells, preferably in mammalian cells (e.g., CHO cells). Also provided herein are host cells comprising such vectors for recombinantly expressing anti-ILT2 antibodies described herein (e.g., human or humanized antibody). In a particular aspect, provided herein are methods for producing an antibody described herein, comprising expressing the antibody from a host cell.

Recombinant expression of an antibody described herein (e.g., a full-length antigen-binding region or antibody or heavy and/or light chain of an antibody described herein) that specifically binds to ILT2 (e.g., human ILT2) generally involves construction of an expression vector containing a polynucleotide that encodes the antibody. Once a polynucleotide encoding an antibody molecule, heavy and/or light chain of an antibody, or a fragment thereof (e.g., heavy and/or light chain variable regions) described herein has been obtained, the vector for the production of the antibody molecule can be produced by recombinant DNA technology using techniques well known in the art. Thus, methods for preparing a protein by expressing a polynucleotide containing an antibody or antibody fragment (e.g., light chain or heavy chain) encoding nucleotide sequence are described herein. Methods which are well known to those skilled in the art can be used to construct expression vectors containing an antibody or antibody fragment (e.g., light chain or heavy chain) coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding containing an antibody molecule described herein, a heavy or light chain of an antibody, a heavy or light chain variable region of an antibody or a fragment thereof, or a heavy or light chain CDR, operably linked to a promoter. Such vectors can, for example, include the nucleotide sequence encoding the constant region of the antibody molecule (see, e.g., International Publication Nos. WO 86/05807 and WO 89/01036; and U.S. Pat. No. 5,122,464, which are herein incorporated by reference in their entireties) and variable regions of the antibody can be cloned into such a vector for expression of the entire heavy, the entire light chain, or both the entire heavy and light chains.

In certain embodiments, a vector comprises a polynucleotide encoding a VH, VL, heavy chain, and/or light chain of an antibody described herein. In another embodiment, a vector comprises a polynucleotide encoding the VH and the VL of an antibody described herein. In another embodiment, a vector comprises a polynucleotide encoding the heavy chain and the light chain of an antibody described herein.

An expression vector can be transferred to a cell (e.g., host cell) by conventional techniques and the resulting cells can then be cultured by conventional techniques to produce containing an antibody described herein or a fragment thereof. Thus, provided herein are host cells containing a polynucleotide encoding containing an antibody described herein or fragments thereof, or a heavy or light chain thereof, or fragment thereof, or a single-chain antibody described herein, operably linked to a promoter for expression of such sequences in the host cell.

In certain embodiments, a host cell comprises a polynucleotide encoding the VH and VL of an antibody described herein. In another embodiment, a host cell comprises a vector comprising a polynucleotide encoding the VH and VL of an antibody described herein. In another embodiment, a host cell comprises a first polynucleotide encoding the VH of an antibody described herein, and a second polynucleotide encoding the VL of an antibody described herein. In another embodiment, a host cell comprises a first vector comprising a first polynucleotide encoding the VH of an antibody described herein, and a second vector comprising a second polynucleotide encoding the VL of an antibody described herein.

In specific embodiments, a heavy chain/heavy chain variable region expressed by a first cell is associated with a light chain/light chain variable region of a second cell to form an anti-ILT2 (e.g., human ILT2) antibody described herein. In certain embodiments, provided herein is a population of host cells comprising such first host cell and such second host cell.

In certain embodiments, provided herein is a population of vectors comprising a first vector comprising a polynucleotide encoding a light chain/light chain variable region of an anti-ILT2 (e.g., human ILT2) antibody described herein, and a second vector comprising a polynucleotide encoding a heavy chain/heavy chain variable region of an anti-ILT2 (e.g., human ILT2) antibody described herein.

A variety of host-expression vector systems can be utilized to express antibody molecules described herein (see, e.g., U.S. Pat. No. 5,807,715, which is herein incorporated by reference in its entirety). Such host-expression systems represent vehicles by which the coding sequences of interest can be produced and subsequently purified, but also represent cells which can, when transformed or transfected with the appropriate nucleotide coding sequences, express an antibody molecule described herein in situ. These include but are not limited to microorganisms such as bacteria (e.g., E. coli and B. subtilis) transformed with, e.g., recombinant bacteriophage DNA, plasmid DNA or cosmid DNA expression vectors containing antibody coding sequences; yeast (e.g., Saccharomyces and Pichia) transformed with, e.g., recombinant yeast expression vectors containing antibody coding sequences; insect cell systems infected with, e.g., recombinant virus expression vectors (e.g., baculovirus) containing antibody coding sequences; plant cell systems (e.g., green algae such as Chlamydomonas reinhardtii) infected with, e.g., recombinant virus expression vectors (e.g., cauliflower mosaic virus, CaMV; tobacco mosaic virus, TMV) or transformed with, e.g., recombinant plasmid expression vectors (e.g., Ti plasmid) containing antibody coding sequences; or mammalian cell systems (e.g., COS (e.g., COS1 or COS), CHO, BHK, MDCK, HEK 293, NS0, PER.C6, VERO, CRL7O3O, HsS78Bst, HeLa, and NIH 3T3, HEK-293T, HepG2, SP210, R1.1, B-W, L-M, BSC1, BSC40, YB/20 and BMT10 cells) harboring, e.g., recombinant expression constructs containing promoters derived from the genome of mammalian cells (e.g., metallothionein promoter) or from mammalian viruses (e.g., the adenovirus late promoter; the vaccinia virus 7.5K promoter). In a specific embodiment, cells for expressing antibodies described herein are Chinese hamster ovary (CHO) cells, for example CHO cells from the CHO GS System™ (Lonza). In certain embodiments, the heavy chain and/or light chain of an antibody produced by a CHO cell may have an N-terminal glutamine or glutamate residue replaced by pyroglutamate. In certain embodiments, cells for expressing antibodies described herein are human cells, e.g., human cell lines. In a specific embodiment, a mammalian expression vector is pOptiVEC™ or pcDNA3.3. In certain embodiments, bacterial cells such as Escherichia coli, or eukaryotic cells (e.g., mammalian cells), especially for the expression of whole recombinant antibody molecule, are used for the expression of a recombinant antibody molecule. For example, mammalian cells such as CHO cells, in conjunction with a vector such as the major intermediate early gene promoter element from human cytomegalovirus, are an effective expression system for antibodies (Foecking M K & Hofstetter H (1986) Gene 45:101-5; and Cockett M I et al., (1990) Biotechnology 8 (7): 662-7, each of which is herein incorporated by reference in its entirety). In certain embodiments, antibodies described herein are produced by CHO cells or NS0 cells. In a specific embodiment, the expression of nucleotide sequences encoding antibodies described herein which specifically bind to ILT2 (e.g., human ILT2) is regulated by a constitutive promoter, inducible promoter, or tissue specific promoter.

In bacterial systems, a number of expression vectors can be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified can be desirable. Such vectors include, but are not limited to, the E. coli expression vector pUR278 (Ruether U & Mueller-Hill B (1983) EMBO J 2:1791-1794), in which the coding sequence can be ligated individually into the vector in frame with the lac Z coding region so that a fusion protein is produced; pIN vectors (Inouye S & Inouye M (1985) Nuc Acids Res 13:3101-3109; Van Heeke G & Schuster S M (1989) J Biol Chem 24:5503-5509); and the like, all of which are herein incorporated by reference in their entireties. For example, pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV), for example, can be used as a vector to express foreign genes. The virus grows in Spodoptera frugiperda cells. The coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the molecule in infected hosts (e.g., see Logan J & Shenk T (1984) PNAS 81 (12): 3655-9, which is herein incorporated by reference in its entirety). Specific initiation signals can also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bitter G et al., (1987) Methods Enzymol. 153:516-544, which is herein incorporated by reference in its entirety).

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20 and T47D, NS0 (a murine myeloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells. In certain embodiments, anti-ILT2 (e.g., human ILT2) antibodies described herein are produced in mammalian cells, such as CHO cells.

In a specific embodiment, the antibodies described herein have reduced fucose content or no fucose content. Such antibodies can be produced using techniques known one skilled in the art. For example, the antibodies can be expressed in cells deficient or lacking the ability of to fucosylate. In a specific example, cell lines with a knockout of both alleles of α1,6-fucosyltransferase can be used to produce antibodies with reduced fucose content. The Potelligent® system (Lonza) is an example of such a system that can be used to produce antibodies with reduced fucose content.

For long-term, high-yield production of recombinant proteins, stable expression cells can be generated. For example, cell lines which stably express an anti-ILT2 (e.g., human ILT2) antibody described herein can be engineered. In specific embodiments, a cell provided herein stably expresses a light chain/light chain variable region and a heavy chain/heavy chain variable region which associate to form an antigen-binding region or an antibody described herein.

In certain aspects, rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA/polynucleotide, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express an anti-ILT2 (e.g., human ILT2) described herein or a fragment thereof. Such engineered cell lines can be particularly useful in screening and evaluation of compositions that interact directly or indirectly with the antibody molecule.

A number of selection systems can be used, including but not limited to the herpes simplex virus thymidine kinase (Wigler M et al., (1977) Cell 11 (1): 223-32), hypoxanthineguanine phosphoribosyltransferase (Szybalska E H & Szybalski W (1962) PNAS 48 (12): 2026-2034) and adenine phosphoribosyltransferase (Lowy I et al., (1980) Cell 22 (3): 817-23) genes in tk-, hgprt- or aprt-cells, respectively, all of which are herein incorporated by reference in their entireties. Also, antimetabolite resistance can be used as the basis of selection for the following genes: dhfr, which confers resistance to methotrexate (Wigler M et al., (1980) PNAS 77 (6): 3567-70; O'Hare K et al., (1981) PNAS 78:1527-31); gpt, which confers resistance to mycophenolic acid (Mulligan R C & Berg P (1981) PNAS 78 (4): 2072-6); neo, which confers resistance to the aminoglycoside G-418 (Wu G Y & Wu C H (1991) Biotherapy 3:87-95; Tolstoshev P (1993) Ann Rev Pharmacol Toxicol 32:573-596; Mulligan R C (1993) Science 260:926-932; and Morgan R A & Anderson W F (1993) Ann Rev Biochem 62:191-217; Nabel G J & Felgner P L (1993) Trends Biotechnol 11 (5): 211-5); and hygro, which confers resistance to hygromycin (Santerre R F et al., (1984) Gene 30 (1-3): 147-56), all of which are herein incorporated by reference in their entireties. Methods commonly known in the art of recombinant DNA technology can be routinely applied to select the desired recombinant clone and such methods are described, for example, in Ausubel F M et al., (eds.), Current Protocols in Molecular Biology, John Wiley & Sons, NY (1993); Kriegler M, Gene Transfer and Expression, A Laboratory Manual, Stockton Press, NY (1990); and in Chapters 12 and 13, Dracopoli N C et al., (eds.), Current Protocols in Human Genetics, John Wiley & Sons, NY (1994); Colbère-Garapin F et al., (1981) J Mol Biol 150:1-14, all of which are herein incorporated by reference in their entireties.

The expression levels of an antibody molecule can be increased by vector amplification (for a review, see Bebbington C R & Hentschel C C G, The use of vectors based on gene amplification for the expression of cloned genes in mammalian cells in DNA cloning, Vol. 3 (Academic Press, New York, 1987), which is herein incorporated by reference in its entirety). When a marker in the vector system is amplifiable, increase in the level of inhibitor present in culture of host cell will increase the number of copies of the marker gene. Since the amplified region is associated with the gene of interest, production of the protein will also increase (Crouse G F et al., (1983) Mol Cell Biol 3:257-66, which is herein incorporated by reference in its entirety).

The host cell can be co-transfected with two or more expression vectors described herein, the first vector encoding a heavy chain derived polypeptide and the second vector encoding a light chain derived polypeptide. The two vectors can contain identical selectable markers which enable equal expression of heavy and light chain polypeptides. The host cells can be co-transfected with different amounts of the two or more expression vectors. For example, host cells can be transfected with any one of the following ratios of a first expression vector and a second expression vector: about 1:1, 1:2, 1:3, 1:4, 1:5, 1:6, 1:7, 1:8, 1:9, 1:10, 1:12, 1:15, 1:20, 1:25, 1:30, 1:35, 1:40, 1:45, or 1:50.

Alternatively, a single vector can be used which encodes, and is capable of expressing, both heavy and light chain polypeptides. In such situations, the light chain should be placed before the heavy chain to avoid an excess of toxic free heavy chain (Proudfoot N J (1986) Nature 322:562-565; and Köhler G (1980) PNAS 77:2197-2199, each of which is herein incorporated by reference in its entirety). The coding sequences for the heavy and light chains can comprise cDNA or genomic DNA. The expression vector can be monocistronic or multicistronic. A multicistronic nucleic acid construct can encode 2, 3, 4, 5, 6, 7, 8, 9, 10 or more genes/nucleotide sequences, or in the range of 2-5, 5-10, or 10-20 genes/nucleotide sequences. For example, a bicistronic nucleic acid construct can comprise, in the following order, a promoter, a first gene (e.g., heavy chain of an antibody described herein), and a second gene and (e.g., light chain of an antibody described herein). In such an expression vector, the transcription of both genes can be driven by the promoter, whereas the translation of the mRNA from the first gene can be by a cap-dependent scanning mechanism and the translation of the mRNA from the second gene can be by a cap-independent mechanism, e.g., by an IRES.

Once an antibody molecule described herein has been produced by recombinant expression, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies described herein can be fused to heterologous polypeptide sequences described herein or otherwise known in the art to facilitate purification.

In specific embodiments, an antibody described herein is isolated or purified. In certain embodiments, an isolated antibody is one that is substantially free of other antibodies with different antigenic specificities than the isolated antibody. For example, in certain embodiments, a preparation of an antibody described herein is substantially free of cellular material and/or chemical precursors. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, 5%, 2%, 1%, 0.5%, or 0.1% (by dry weight) of heterologous protein (also referred to herein as a “contaminating protein”) and/or variants of an antibody, for example, different post-translational modified forms of an antibody or other different versions of an antibody (e.g., antibody fragments). When the antibody is recombinantly produced, it is also generally substantially free of culture medium, i.e., culture medium represents less than about 20%, 10%, 2%, 1%, 0.5%, or 0.1% of the volume of the protein preparation. When the antibody is produced by chemical synthesis, it is generally substantially free of chemical precursors or other chemicals, i.e., it is separated from chemical precursors or other chemicals which are involved in the synthesis of the protein. Accordingly, such preparations of the antibody have less than about 30%, 20%, 10%, or 5% (by dry weight) of chemical precursors or compounds other than the antibody of interest. In a specific embodiment, antibodies described herein are isolated or purified.

Anti-ILT2 (e.g., human ILT2) antibodies or fragments thereof can be produced by any method known in the art for the synthesis of proteins or antibodies, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Maniatis T et al., (1982) Molecular Cloning: A Laboratory Manual, Cold Spring Harbor Laboratory Press; Sambrook J et al., (1989), Molecular Cloning: A Laboratory Manual, Second Edition, Cold Spring Harbor Laboratory Press; Sambrook J et al., (2001) Molecular Cloning: A Laboratory Manual, Cold Spring Harbor Laboratory Press, Cold Spring Harbor, NY; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press, all of which are herein incorporated by reference in their entireties.

In a specific embodiment, an antibody described herein is prepared, expressed, created, or isolated by any means that involves creation, e.g., via synthesis, genetic engineering of DNA sequences. In certain embodiments, such an antibody comprises sequences (e.g., DNA sequences or amino acid sequences) that do not naturally exist within the antibody germline repertoire of an animal or mammal (e.g., human) in vivo.

In one aspect, provided herein is a method of making an anti-ILT2 (e.g., human ILT2) antibody comprising culturing a cell or host cell described herein. In certain embodiments, the method is performed in vitro. In a certain aspect, provided herein is a method of making an anti-ILT2 (e.g., human ILT2) antibody comprising expressing (e.g., recombinantly expressing) the antibody using a cell or host cell described herein (e.g., a cell or a host cell comprising polynucleotides encoding an antibody described herein). In certain embodiments, the cell is an isolated cell. In certain embodiments, the exogenous polynucleotides have been introduced into the cell. In certain embodiments, the method further comprises the step of purifying the antibody obtained from the cell or host cell.

In certain embodiments, an antibody is produced by expressing in a cell a polynucleotide encoding the VH and VL of an antibody described herein under suitable conditions so that the polynucleotides are expressed and the antibody is produced. In another embodiment, an antibody is produced by expressing in a cell a polynucleotide encoding the heavy chain and light chain of an antibody described herein under suitable conditions so that the polynucleotides are expressed and the antibody is produced. In certain embodiments, an antibody is produced by expressing in a cell a first polynucleotide encoding the VH of an antibody described herein, and a second polynucleotide encoding the VL of an antibody described herein, under suitable conditions so that the polynucleotides are expressed and the antibody is produced. In certain embodiments, an antibody is produced by expressing in a cell a first polynucleotide encoding the heavy chain of an antibody described herein, and a second polynucleotide encoding the light chain of an antibody described herein, under suitable conditions so that the polynucleotides are expressed and the antibody is produced.

Methods for producing polyclonal antibodies are known in the art (see, for example, Chapter 11 in: Short Protocols in Molecular Biology, (2002) 5^{th} Ed., Ausubel F M et al., eds., John Wiley and Sons, New York, which is herein incorporated by reference in its entirety).

Monoclonal antibodies can be prepared using a wide variety of techniques known in the art, including the use of hybridoma, recombinant, and phage display technologies, or a combination thereof. For example, monoclonal antibodies can be produced using hybridoma techniques, including those known in the art and taught, for example, in Harlow E & Lane D, Antibodies: A Laboratory Manual, (Cold Spring Harbor Laboratory Press, 2^{nd} ed. 1988); Hammerling G J et al., in: Monoclonal Antibodies and T-Cell Hybridomas 563 681 (Elsevier, N. Y., 1981), each of which is herein incorporated by reference in its entirety. The term “monoclonal antibody” as used herein is not limited to antibodies produced through hybridoma technology. For example, monoclonal antibodies can be produced recombinantly from host cells exogenously expressing an antibody described herein or a fragment thereof, for example, light chain and/or heavy chain of such antibody.

In specific embodiments, a “monoclonal antibody,” as used herein, is an antibody produced by a single cell (e.g., hybridoma or host cell producing a recombinant antibody), wherein the antibody specifically binds to ILT2 (e.g., human ILT2) as determined, e.g., by ELISA or other antigen-binding or competitive binding assay known in the art or in the examples provided herein. In certain embodiments, a monoclonal antibody can be a chimeric antibody or a humanized antibody. In certain embodiments, a monoclonal antibody is a monovalent antibody or multivalent (e.g., bivalent) antibody. In certain embodiments, a monoclonal antibody is a monospecific or multispecific antibody (e.g., bispecific antibody). Monoclonal antibodies described herein can, for example, be made by the hybridoma method as described in Kohler G & Milstein C (1975) Nature 256:495, which is herein incorporated by reference in its entirety, or can, e.g., be isolated from phage libraries using the techniques as described herein, for example. Other methods for the preparation of clonal cell lines and of monoclonal antibodies expressed thereby are well known in the art (see, for example, Chapter 11 in: Short Protocols in Molecular Biology, (2002) 5th Ed., Ausubel F M et al., supra).

As used herein, an antibody binds to an antigen multivalently (e.g., bivalently) when the antibody comprises at least two (e.g., two or more) monovalent binding regions, each monovalent binding region capable of binding to an epitope on the antigen. Each monovalent binding region can bind to the same or different epitopes on the antigen.

Methods for producing and screening for specific antibodies using hybridoma technology are routine and well known in the art. For example, in the hybridoma method, a mouse or other appropriate host animal, such as a sheep, goat, rabbit, rat, hamster, or macaque monkey, is immunized to elicit lymphocytes that produce or are capable of producing antibodies that will specifically bind to the protein (e.g., ILT2 (e.g., human ILT2)) used for immunization. Alternatively, lymphocytes may be immunized in vitro. Lymphocytes then are fused with myeloma cells using a suitable fusing agent, such as polyethylene glycol, to form a hybridoma cell (Goding J W (Ed), Monoclonal Antibodies: Principles and Practice, pp. 59-103 (Academic Press, 1986), herein incorporated by reference in its entirety). Additionally, a RIMMS (repetitive immunization multiple sites) technique can be used to immunize an animal (Kilpatrick K E et al., (1997) Hybridoma 16:381-9, herein incorporated by reference in its entirety).

In certain embodiments, mice (or other animals, such as rats, monkeys, donkeys, pigs, sheep, hamster, or dogs) can be immunized with an antigen (e.g., ILT2 (e.g., human ILT2)) and once an immune response is detected, e.g., antibodies specific for the antigen are detected in the mouse serum, the mouse spleen is harvested and splenocytes isolated. The splenocytes are then fused by well-known techniques to any suitable myeloma cells, for example, cells from cell line SP20 available from the American Type Culture Collection (ATCC®) (Manassas, VA), to form hybridomas. Hybridomas are selected and cloned by limited dilution. In certain embodiments, lymph nodes of the immunized mice are harvested and fused with NS0 myeloma cells.

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Specific embodiments employ myeloma cells that fuse efficiently, support stable high-level production of antibody by the selected antibody-producing cells, and are sensitive to a medium such as HAT medium. Among these myeloma cell lines are murine myeloma lines, such as the NS0 cell line or those derived from MOPC-21 and MPC-11 mouse tumors available from the Salk Institute Cell Distribution Center, San Diego, CA, USA, and SP-2 or X63-Ag8.653 cells available from the American Type Culture Collection, Rockville, MD, USA. Human myeloma and mouse-human heteromyeloma cell lines also have been described for the production of human monoclonal antibodies (Kozbor D (1984) J Immunol 133:3001-5; Brodeur et al., Monoclonal Antibody Production Techniques and Applications, pp. 51-63 (Marcel Dekker, Inc., New York, 1987), each of which is herein incorporated by reference in its entirety).

Culture medium in which hybridoma cells are growing is assayed for production of monoclonal antibodies directed against ILT2 (e.g., human ILT2). The binding specificity of monoclonal antibodies produced by hybridoma cells is determined by methods known in the art, for example, immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subcloned by limiting dilution procedures and grown by standard methods (Goding J W (Ed), Monoclonal Antibodies: Principles and Practice, supra). Suitable culture media for this purpose include, for example, D-MEM or RPMI 1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Antibodies described herein include, e.g., antibody fragments which recognize ILT2 (e.g., human ILT2), and can be generated by any technique known to those of skill in the art. For example, Fab and F(ab′)₂ fragments described herein can be produced by proteolytic cleavage of immunoglobulin molecules, using enzymes such as papain (to produce Fab fragments) or pepsin (to produce F(ab′)₂ fragments). A Fab fragment corresponds to one of the two identical arms of an antibody molecule and contains the complete light chain paired with the VH and CH1 domains of the heavy chain. A F(ab′)₂ fragment contains the two antigen-binding arms of an antibody molecule linked by disulfide bonds in the hinge region.

Further, the antibodies described herein can also be generated using various phage display methods known in the art. In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with a scFv linker by PCR and cloned into a phagemid vector. The vector is electroporated in E. coli, and the E. coli is infected with helper phage. Phage used in these methods are typically filamentous phage, including fd and M13, and the VH and VL domains are usually recombinantly fused to either the phage gene III or gene VIII. Phage expressing an antigen-binding region that binds to a particular antigen can be selected or identified with antigen, e.g., using labeled antigen or antigen bound or captured to a solid surface or bead. Examples of phage display methods that can be used to make the antibodies described herein include those disclosed in Brinkman U et al., (1995) J Immunol Methods 182:41-50; Ames R S et al., (1995) J Immunol Methods 184:177-186; Kettleborough C A et al., (1994) Eur J Immunol 24:952-958; Persic L et al., (1997) Gene 187:9-18; Burton D R & Barbas C F (1994) Advan Immunol 57:191-280; PCT Application No. PCT/GB91/001134; International Publication Nos. WO 90/02809, WO 91/10737, WO 92/01047, WO 92/18619, WO 93/1 1236, WO 95/15982, WO 95/20401, and WO 97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743, and 5,969,108, all of which are herein incorporated by reference in their entireties.

As described in the above references, after phage selection, the antibody coding regions from the phage can be isolated and used to generate whole antibodies, including human antibodies, or any other desired antigen-binding fragment, and expressed in any desired host, including mammalian cells, insect cells, plant cells, yeast, and bacteria, e.g., as described below. Techniques to recombinantly produce antibody fragments such as Fab, Fab′ and F(ab′)₂ fragments can also be employed using methods known in the art such as those disclosed in PCT publication No. WO 92/22324; Mullinax R L et al., (1992) BioTechniques 12 (6): 864-9; Sawai H et al., (1995) Am J Reprod Immunol 34:26-34; and Better M et al., (1988) Science 240:1041-1043, all of which are herein incorporated by reference in their entireties.

In certain embodiments, to generate whole antibodies, PCR primers including VH or VL nucleotide sequences, a restriction site, and a flanking sequence to protect the restriction site can be used to amplify the VH or VL sequences from a template, e.g., scFv clones. Utilizing cloning techniques known to those of skill in the art, the PCR amplified VH domains can be cloned into vectors expressing a VH constant region, and the PCR amplified VL domains can be cloned into vectors expressing a VL constant region, e.g., human kappa or lambda constant regions. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

A chimeric antibody is a molecule in which different portions of the antibody are derived from different immunoglobulin molecules. For example, a chimeric antibody can contain a variable region of a mouse or rat monoclonal antibody fused to a constant region of a human antibody. Methods for producing chimeric antibodies are known in the art. See, e.g., Morrison S L (1985) Science 229:1202-7; Oi V T & Morrison S L (1986) BioTechniques 4:214-221; Gillies S D et al., (1989) J Immunol Methods 125:191-202; and U.S. Pat. Nos. 5,807,715, 4,816,567, 4,816,397, and 6,331,415, all of which are herein incorporated by reference in their entireties.

A humanized antibody is capable of binding to a predetermined antigen and which comprises a framework region having substantially the amino acid sequence of a human immunoglobulin and CDRs having substantially the amino acid sequence of a non-human immunoglobulin (e.g., a murine immunoglobulin). In certain embodiments, a humanized antibody also comprises at least a portion of an immunoglobulin constant region (Fc), typically that of a human immunoglobulin. The antibody also can include the CH1, hinge, CH2, CH3, and CH4 regions of the heavy chain. A humanized antibody can be selected from any class of immunoglobulins, including IgM, IgG, IgD, IgA, and IgE, and any isotype, including IgG₁, IgG₂, IgG₃, and IgG₄. Humanized antibodies can be produced using a variety of techniques known in the art, including but not limited to, CDR-grafting (European Patent No. EP 239400; International Publication No. WO 91/09967; and U.S. Pat. Nos. 5,225,539, 5,530,101, and 5,585,089), veneering or resurfacing (European Patent Nos. EP 592106 and EP 519596; Padlan E A (1991) Mol Immunol 28 (4/5): 489-498; Studnicka G M et al., (1994) Prot Engineering 7 (6): 805-814; and Roguska M A et al., (1994) PNAS 91:969-973), chain shuffling (U.S. Pat. No. 5,565,332), and techniques disclosed in, e.g., U.S. Pat. Nos. 6,407,213, 5,766,886, International Publication No. WO 93/17105; Tan P et al., (2002) J Immunol 169:1119-25; Caldas C et al., (2000) Protein Eng. 13 (5): 353-60; Morea V et al., (2000) Methods 20 (3): 267-79; Baca M et al., (1997) J Biol Chem 272 (16): 10678-84; Roguska M A et al., (1996) Protein Eng 9 (10): 895 904; Couto J R et al., (1995) Cancer Res. 55 (23 Supp): 5973s-5977s; Couto J R et al., (1995) Cancer Res 55 (8): 1717-22; Sandhu J S (1994) Gene 150 (2): 409-10; and Pedersen J T et al., (1994) J Mol Biol 235 (3): 959-73, all of which are herein incorporated by reference in their entireties. See also, U.S. Application Publication No. US 2005/0042664 A1 (Feb. 24, 2005), which is herein incorporated by reference in its entirety.

Methods for making multispecific antibodies (e.g., bispecific antibodies) have been described, see, for example, U.S. Pat. Nos. 7,951,917; 7,183,076; 8,227,577; 5,837,242; 5,989,830; 5,869,620; 6,132,992; and 8,586,713, all of which are herein incorporated by reference in their entireties.

Bispecific, bivalent antibodies, and methods of making them, are described, for instance in U.S. Pat. Nos. 5,731,168, 5,807,706, 5,821,333, and U.S. Appl. Publ. Nos. 2003/020734 and 2002/0155537; each of which is herein incorporated by reference in its entirety. Bispecific tetravalent antibodies, and methods of making them are described, for instance, in Int. Appl. Publ. Nos. WO 02/096948 and WO 00/44788, the disclosures of both of which are herein incorporated by reference in its entirety. See generally, Int. Appl. Publ. Nos. WO 93/17715, WO 92/08802, WO 91/00360, and WO 92/05793; Tutt et al., J. Immunol. 147:60-69 (1991); U.S. Pat. Nos. 4,474,893; 4,714,681; 4,925,648; 5,573,920; and 5,601,819; and Kostelny et al., J. Immunol. 148:1547-1553 (1992); each of which is herein incorporated by reference in its entirety.

A bispecific antibody as described herein can be generated according to the DuoBody technology platform (Genmab A/S) as described, e.g., in International Publication Nos. WO 2011/131746, WO 2011/147986, WO 2008/119353, and WO 2013/060867, and in Labrijn A F et al., (2013) PNAS 110 (13): 5145-5150. The DuoBody technology can be used to combine one half of a first monospecific antibody, or first antigen-binding region, containing two heavy and two light chains with one half of a second monospecific antibody, or second antigen-binding region, containing two heavy and two light chains. The resultant heterodimer contains one heavy chain and one light chain from the first antibody, or first antigen-binding region, paired with one heavy chain and one light chain from the second antibody, or second antigen-binding region. When both of the monospecific antibodies, or antigen-binding regions, recognize different epitopes on different antigens, the resultant heterodimer is a bispecific antibody.

The DuoBody technology requires that each of the monospecific antibodies, or antigen-binding regions includes a heavy chain constant region with a single point mutation in the CH3 domain. The point mutations allow for a stronger interaction between the CH3 domains in the resultant bispecific antibody than between the CH3 domains in either of the monospecific antibodies, or antigen-binding regions. The single point mutation in each monospecific antibody, or antigen-binding region, is at residue 366, 368, 370, 399, 405, 407, or 409, numbered according to the EU numbering system, in the CH3 domain of the heavy chain constant region, as described, e.g., in International Publication No. WO 2011/131746. Moreover, the single point mutation is located at a different residue in one monospecific antibody, or antigen-binding region, as compared to the other monospecific antibody, or antigen-binding region. For example, one monospecific antibody, or antigen-binding region, can comprise the mutation F405L (i.e., a mutation from phenylalanine to leucine at residue 405), while the other monospecific antibody, or antigen-binding region, can comprise the mutation K409R (i.e., a mutation from lysine to arginine at residue 409), numbered according to the EU numbering system. The heavy chain constant regions of the monospecific antibodies, or antigen-binding regions, can be an IgG₁, IgG₂, IgG₃, or IgG₄ isotype (e.g., a human IgG₁ isotype), and a bispecific antibody produced by the DuoBody technology can retain Fc-mediated effector functions.

Another method for generating bispecific antibodies has been termed the “knobs-into-holes”strategy (see, e.g., Intl. Publ. WO2006/028936). The mispairing of Ig heavy chains is reduced in this technology by mutating selected amino acids forming the interface of the CH3 domains in IgG. At positions within the CH3 domain at which the two heavy chains interact directly, an amino acid with a small side chain (hole) is introduced into the sequence of one heavy chain and an amino acid with a large side chain (knob) into the counterpart interacting residue location on the other heavy chain. In some embodiments, compositions of the disclosure have immunoglobulin chains in which the CH3 domains have been modified by mutating selected amino acids that interact at the interface between two polypeptides so as to preferentially form a bispecific antibody. The bispecific antibodies can be composed of immunoglobulin chains of the same subclass (e.g., IgG₁ or IgG₃) or different subclasses (e.g., IgG₁ and IgG₃, or IgG₃ and IgG₄).

Bispecific antibodies can, in some instances contain, IgG₄ and IgG₁, IgG₄ and IgG₂, IgG₄ and IgG₃, or IgG₁ and IgG₃ chain heterodimers. Such heterodimeric heavy chain antibodies can routinely be engineered by, for example, modifying selected amino acids forming the interface of the CH3 domains in human IgG₄ and the IgG₁ or IgG₃, so as to favor heterodimeric heavy chain formation.

In certain embodiments, an antibody described herein, which binds to the same epitope of ILT2 (e.g., human ILT2) as an anti-ILT2 (e.g., human ILT2) antibody described herein, is a human antibody. In certain embodiments, an antibody described herein, which competitively blocks (e.g., in a dose-dependent manner) any one of the antibodies described herein, from binding to ILT2 (e.g., human ILT2), is a human antibody. Human antibodies can be produced using any method known in the art. For example, transgenic mice which are incapable of expressing functional endogenous immunoglobulins, but which can express human immunoglobulin genes, can be used. In particular, the human heavy and light chain immunoglobulin gene complexes can be introduced randomly or by homologous recombination into mouse embryonic stem cells. Alternatively, the human variable region, constant region, and diversity region can be introduced into mouse embryonic stem cells in addition to the human heavy and light chain genes. The mouse heavy and light chain immunoglobulin genes can be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of an antigen (e.g., ILT2 (e.g., human ILT2)). Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM, and IgE antibodies. For an overview of this technology for producing human antibodies, see Lonberg N & Huszar D (1995) Int Rev Immunol 13:65-93, herein incorporated by reference in its entirety. For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, all of which are herein incorporated by reference in their entireties. Examples of mice capable of producing human antibodies include the XenoMouse™ (Abgenix, Inc.; U.S. Pat. Nos. 6,075,181 and 6,150,184), the HuAb-Mouse™ (Medarex, Inc./Gen Pharm; U.S. Pat. Nos. 5,545,806 and 5,569,825), the Trans Chromo Mouse™ (Kirin) and the KM Mouse™ (Medarex/Kirin), all of which are herein incorporated by reference in their entireties.

Human antibodies that specifically bind to ILT2 (e.g., human ILT2) can be made by a variety of methods known in the art, including the phage display methods described above using antibody libraries derived from human immunoglobulin sequences. See also, U.S. Pat. Nos. 4,444,887, 4,716,111, and 5,885,793; and International Publication Nos. WO 98/46645, WO 98/50433, WO 98/24893, WO 98/16654, WO 96/34096, WO 96/33735, and WO 91/10741, all of which are herein incorporated by reference in their entireties.

In certain embodiments, human antibodies can be produced using mouse-human hybridomas. For example, human peripheral blood lymphocytes transformed with Epstein-Barr virus (EBV) can be fused with mouse myeloma cells to produce mouse-human hybridomas secreting human monoclonal antibodies, and these mouse-human hybridomas can be screened to determine ones which secrete human monoclonal antibodies that specifically bind to a target antigen (e.g., ILT2 (e.g., human ILT2)). Such methods are known and are described in the art, see, e.g., Shinmoto H et al., (2004) Cytotechnology 46:19-23; Naganawa Y et al., (2005) Human Antibodies 14:27-31, each of which is herein incorporated by reference in its entirety.

### Kits

Also provided are kits comprising one or more antibodies described herein, or pharmaceutical compositions or conjugates thereof. In a specific embodiment, provided herein is a pharmaceutical pack or kit comprising one or more containers filled with one or more of the ingredients of the pharmaceutical compositions described herein, such as one or more antibodies provided herein. In certain embodiments, the kits contain a pharmaceutical composition described herein and any prophylactic or therapeutic agent, such as those described herein. In certain embodiments, the kits may contain a T cell mitogen, such as, e.g., phytohaemagglutinin (PHA) and/or phorbol myristate acetate (PMA), or a TCR complex stimulating antibody, such as an anti-CD3 antibody and anti-CD28 antibody. Optionally associated with such container(s) can be a notice in the form prescribed by a governmental agency regulating the manufacture, use or sale of pharmaceuticals or biological products, which notice reflects approval by the agency of manufacture, use or sale for human administration.

Also provided, are kits that can be used in the above methods. In certain embodiments, a kit comprises an antibody described herein, preferably purified antibody, in one or more containers. In a specific embodiment, kits described herein contain a substantially isolated ILT2 (e.g., human ILT2) antigen as a control. In another specific embodiment, the kits described herein further comprise a control antibody which does not react with ILT2 (e.g., human ILT2) antigen. In another specific embodiment, kits described herein contain one or more elements for detecting the binding of an antibody to an ILT2 (e.g., human ILT2) antigen (e.g., the antibody can be conjugated to a detectable substrate such as a fluorescent compound, an enzymatic substrate, a radioactive compound or a luminescent compound, or a second antibody which recognizes the first antibody can be conjugated to a detectable substrate). In specific embodiments, a kit provided herein can include a recombinantly produced or chemically synthesized ILT2 (e.g., human ILT2) antigen. The ILT2 (e.g., human ILT2) antigen provided in the kit can also be attached to a solid support. In a more specific embodiment, the detecting means of the above-described kit includes a solid support to which an ILT2 (e.g., human ILT2) antigen is attached. Such a kit can also include a non-attached reporter-labeled anti-human antibody or anti-mouse/rat antibody. In this embodiment, binding of the antibody to the ILT2 (e.g., human ILT2) antigen can be detected by binding of the said reporter-labeled antibody. In certain embodiments, the present disclosure relates to the use of a kit of the present disclosure for in vitro assaying and/or detecting ILT2 (e.g., human ILT2) antigen in a biological sample.

### EXAMPLES

The following examples are offered by way of illustration and not by way of limitation.

### Example 1: Binding Kinetics and Optimization of Anti-ILT2 Antibodies

This example describes the characterization of antibodies that specifically bind to human ILT2. The amino acid sequences of these antibodies are set forth herein in Table 2.

An anti-ILT2 antibody generated by Mammalian Retrocyte Display was affinity matured by phage display. Variants of this antibody, named BA210, were made to improve properties and optimize the molecule. BA210 variable domains were formatted with human kappa IgG₁, IgG₂, or IgG₄ constant regions, with substitutions for heavy chain aglycosylation (N297A), disulfide bond formation and stabilization (C127S, S228P), and to remove a predicted deamidation site at position 34 (G) and oxidation site at position 105 (M) in the light chain, according to Table 3 below. Additional variants with C-terminal lysine deletions were made for improved manufacturability.

TABLE 3
ILT2 Antibody Variants

| Antibody | Description | Antibody | Description |
|---|---|---|---|
| BA210 | IgG1 | BA220 | G34Y/M105L IgG2 C127S |
| BA211 | IgG1 N297A | BA221 | G34T/M105L IgG4 S228P |
| BA212 | G34T IgG1 N297A | BA222 | G34Y/M105L IgG4 S228P |
| BA213 | M105L IgG1 N297A | BA246 | IgG2 |
| BA214 | G34T/M105L IgG1 N297A | BA247 | IgG2 C127S |
| BA215 | G34Y/M105L IgG1 N297A | BA248 | IgG4 S228P |
| BA216 | G34Y IgG1 N297A | BA249 | G34T/M105L IgG1 |
| BA217 | G34T/M105L IgG2 | BA250 | G34Y/M105L IgG1 |
| BA218 | G34Y/M105L IgG2 | BA251 | G34T/M105L IgG1 N297A-C-term K |
| BA219 | G34T/M105L IgG2 C127S | BA252 | G34T/M105L IgG4 S228P-C-term K |

### Binding to Purified Human ILT2 Protein

The binding affinity of anti-ILT2 antibody variants with IgG1 N297A constant regions to ECD-His tagged human ILT2 haplotypes PE01, PE02, PE03 (see Table 1) was assessed by surface plasmon resonance (SPR). Antibody BA211, containing identified post-translation modification (PTM) risks, was used as positive isotype control, and irrelevant antibody VRC01 was used as negative isotype control.

Briefly, SPR experiments were performed using a Biacore T200 instrument, and the association rate (Kₐ), dissociation rate (K_{d}), and dissociation constant (K_{D}) were calculated from each experiment using a 1:1 binding model with Biacore™ T200 Evaluation Software. Capture of antibodies from the Human Fab Capture kit (28958325, GE Healthcare) to the Series S CM5 sensor chip (29149603, GE Healthcare) was performed using the immobilization wizard for amine coupling from the Biacore™ control panel and following the manufacturer's recommendations included with the kit.

Approximately 3 μg/mL of tested anti-ILT2 antibodies and controls diluted in a running buffer (10 mM HEPES, 150 mM NaCl, 3 mM EDTA, and 0.05% surfactant P20) were captured in individual flow cells of previously prepared anti Human Fab CM5 chip, keeping a single flow cell as a reference. Antibodies were captured using a 30 sec injection at a flow rate of 10 μL/min to reach about 150 resonance units (Rus). Human ILT2 ECD PE01 (diluted in the running buffer at the concentration of 30, 10, 3.3, 1.11, 0.37, 0.12 nM), and human ILT2 ECD PE02 and PE03 (diluted in the running buffer at the concentration of 33.3, 11.1, 3.7, 1.2, 0.41, 0.14 nM), were flowed over the chip surface at a flow rate of 30 μL/min with a 3-min association phase and a 10-min dissociation phase. The sensor chip was regenerated between cycles with a double 30-sec injection of 10 mM glycine, pH 2.1 at 30 μL/min followed by stabilization period of 60s. Sensorgrams were evaluated and fit to a simple Langmuir 1:1 interaction model using the global data analysis option of BIAevaluation 3.1 software. Data quality was verified by visually inspecting deviations and curve fitting, and by evaluating the parameters of Rₘₐₓ, Chi2, and Tc. The binding kinetics are shown in Table 4.

TABLE 4
Kinetic parameters of anti-ILT2 antibody variants binding
to human ILT2 haplotypes PE01, PE02, PE03.

| | ILT2 | | | |
|---|---|---|---|---|
| Antibody | haplotype | Kₐ (1/Ms) | K_{d} (1/s) | K_{D} (pM) |
| BA211 | PE01 | 3.4E+05 | 3.4E−05 | 99 |
| BA212 | PE01 | 3.7E+04 | 3.3E−06 | 89 |
| BA216 | PE01 | 3.6E+05 | 6.7E−05 | 185 |
| BA213 | PE01 | 3.2E+05 | 1.1E−05 | 33 |
| BA214 | PE01 | 3.3E+05 | 2.1E−05 | 64 |
| BA215 | PE01 | 2.8E+05 | 9.6E−07 | 3 |
| BA211 | PE02 | 2.6E+05 | 1.7E−04 | 671 |
| BA212 | PE02 | 2.7E+05 | 2.1E−04 | 783 |
| BA216 | PE02 | 2.3E+05 | 1.4E−04 | 617 |
| BA213 | PE02 | 2.3E+05 | 1.5E−04 | 630 |
| BA214 | PE02 | 2.6E+05 | 1.3E−04 | 513 |
| BA215 | PE02 | 2.3E+05 | 1.2E−04 | 541 |
| BA211 | PE03 | 2.7E+05 | 2.5E−04 | 929 |
| BA212 | PE03 | 4.6E+05 | 4.0E−04 | 875 |
| BA216 | PE03 | 2.7E+05 | 8.5E−05 | 317 |
| BA213 | PE03 | 5.0E+05 | 7.5E−05 | 149 |
| BA214 | PE03 | 5.4E+05 | 1.4E−04 | 257 |
| BA215 | PE03 | 4.9E+05 | 1.1E−04 | 216 |

In another experiment, the binding affinities of anti-ILT2 antibody variants with light chain substitutions and/or different heavy chain constant regions (BA249, BA250, BA214, BA215, BA217, BA218, BA219, BA220, BA221, and BA222) to ECD-His tagged human ILT2 haplotype PE01, were assessed by SPR. Antibodies with identified PTM risks (BA210, BA211, BA246, BA247, and BA248) were used as positive isotype controls. Variants of irrelevant antibody VRC01 (with IgG1, IgG1 N297A, IgG4 S228P, IgG2, or IgG2 C127S constant regions) were used as negative isotype controls. The binding kinetics are shown in Table 5 as an average from two independent measurements.

TABLE 5

| Kinetic parameters of anti-ILT2 antibody Fc | | | |
|---|---|---|---|
| isotypes binding to human ILT2 | | | |
| | ILT2 (PE01) | | |
| Antibody | Kₐ (1/Ms) | K_{d} (1/s) | KD (M) |
| BA210 | 7.1E+05 | 8.9E−05 | 1.5E−10 |
| BA211 | 3.7E+05 | 5.6E−05 | 1.5E−10 |
| BA246 | 3.3E+05 | 7.4E−05 | 2.3E−10 |
| BA247 | 3.3E+05 | 9.7E−05 | 3.1E−10 |
| BA248 | 4.1E+05 | 8.3E−05 | 2.2E−10 |
| BA249 | 3.8E+05 | 4.8E−05 | 1.3E−10 |
| BA214 | 3.9E+05 | 6.8E−05 | 1.9E−10 |
| BA217 | 3.4E+05 | 5.4E−05 | 1.7E−10 |
| BA219 | 3.5E+05 | 4.8E−05 | 1.4E−10 |
| BA221 | 3.7E+05 | 7.3E−05 | 2.0E−10 |
| BA250 | 4.3E+05 | 4.8E−05 | 1.1E−10 |
| BA215 | 4.3E+05 | 5.4E−05 | 1.3E−10 |
| BA218 | 3.9E+05 | 7.0E−05 | 1.9E−10 |
| BA220 | 4.2E+05 | 5.7E−05 | 1.4E−10 |
| BA222 | 9.7E+05 | 5.1E−05 | 6.5E−11 |

In another experiment, the binding affinities of anti-ILT2 antibody variants BA214, BA221, BA251 and BA252 to ECD-His tagged human ILT2 haplotypes PE01, PE02, and PE03, and ECD-His tagged cynomolgus monkey ILT2 orthologue protein, variant 1, ECD-His tagged cynomolgus monkey ILT2 orthologue protein, variant 2, ECD-His tagged cynomolgus monkey ILT2 orthologue protein, variant 3, ECD-His tagged rhesus monkey ILT2 orthologue protein, variant 1, ECD-His tagged rhesus monkey ILT2 orthologue protein, variant 2, ECD-His tagged African green monkey (AGM) ILT2 orthologue protein, and ECD-His tagged mouse PirB protein (R&D Systems, 2754PB050) were assessed by SPR. Irrelevant antibody VRC01 with IgG4 S228P constant region was used as negative isotype control. The binding kinetics (Kₐ, K_{d} and K_{D}) or dissociation equilibrium constant (KD) are shown in Table 6 and Table 7.

TABLE 6
Kinetic Parameters for anti-ILT2 antibodies binding
to human ILT2 haplotypes

| Antibody | ILT2 haplotype | Kₐ (1/Ms) | K_{d} (1/s) | K_{D} (M) |
|---|---|---|---|---|
| BA214 | PE01 | 5.2E+05 | 6.9E−05 | 1.3E−10 |
| BA221 | PE01 | 5.2E+05 | 8.7E−05 | 1.7E−10 |
| BA251 | PE01 | 5.4E+05 | 5.7E−05 | 1.1E−10 |
| BA252 | PE01 | 5.2E+05 | 7.1E−05 | 1.4E−10 |
| Control | PE01 | NBD | NBD | NBD |
| BA214 | PE02 | 4.6E+05 | 2.2E−04 | 4.7E−10 |
| BA221 | PE02 | 4.8E+05 | 2.3E−04 | 4.8E−10 |
| BA251 | PE02 | 4.8E+05 | 2.0E−04 | 4.1E−10 |
| BA252 | PE02 | 4.6E+05 | 2.0E−04 | 4.4E−10 |
| Control | PE02 | NBD | NBD | NBD |
| BA214 | PE03 | 4.2E+05 | 2.6E−04 | 6.3E−10 |
| BA221 | PE03 | 4.4E+05 | 2.9E−04 | 6.6E−10 |
| BA251 | PE03 | 4.3E+05 | 2.4E−04 | 5.6E−10 |
| BA252 | PE03 | 4.2E+05 | 2.5E−04 | 5.9E−10 |
| Control | PE03 | NBD | NBD | NBD |

NBD = no binding detected up to 100 nM

TABLE 7
Binding of anti-ILT2 antibodies to non-human primate
ILT2 orthologs and mouse PirB

| | Binding affinity KD (M) | | | | |
|---|---|---|---|---|---|
| Species | BA214 | BA221 | BA251 | BA252 | Control |
| Cyno ILT2 variant 1 | NBD | NBD | NBD | NBD | NBD |
| Cyno ILT2 variant 2 | NBD | NBD | NBD | NBD | NBD |
| Cyno ILT2 variant 3 | 2.4E−06 | 2.8E−06 | 1.7E−06 | 1.9E−06 | NBD |
| Rhesus ILT2 variant 1 | NBD | NBD | NBD | NBD | NBD |
| Rhesus ILT2 variant 2 | NBD | NBD | NBD | NBD | NBD |
| AGM ILT2 | NBD | NBD | NBD | NBD | NBD |
| Mouse PirB | NBD | NBD | NBD | NBD | NBD |

NBD = no binding detected (up to 10 RU at 5 μM concentration of receptor)

### Specific Binding to ILT2

Binding affinities of anti-ILT2 antibodies BA214, BA221, BA251, and BA252 to recombinant human LILRA1/LIR-6 (Sino Biological 17220-H08H-100), LILRA2/ILT1 (R&D Systems 9040-T4-050), LILRA3/ILT6 (Sino Biological 13549-H08H-100), LILRA4/ILT7 (Sino Biological 16058-H08H-50), LILRA5/ILT11 (Sino Biological 16059-H08H-100), LILRA6/ILT8 (Sino Biological 29813-H08H-50), LILRB1/ILT2 PE01 (SEQ ID NO: 44), LILRB2/ILT4 (R&D Systems 8429-T4-050), LILRB3/ILT5 (R&D Systems 9159-T5-050), LILRB4/ILT3 (Sino Biological 16742-H08H), and LILRB5/LIR-8 (Sino Biological 17221-H08H-100) were assessed by SPR as above. Irrelevant antibody VRC01 IgG4 S228P was used as a negative isotype control.

Results showed binding to ILT2 at the kinetic parameters indicated in Table 8. No binding at physiologically relevant levels was detected for any other LILR family members, indicating specific binding to ILT2.

TABLE 8

| Kinetic parameters of anti-ILT2 antibodies binding to ILT2 | | | |
|---|---|---|---|
| | ILT2 | | |
| | Kₐ (1/Ms) | K_{d} (1/s) | K_{D} (M) |
| BA214 | 4.8E+05 | 1.2E−04 | 2.6E−10 |
| BA221 | 4.6E+05 | 2.1E−04 | 4.5E−10 |
| BA251 | 5.0E+05 | 1.2E−04 | 2.3E−10 |
| BA252 | 4.8E+05 | 1.1E−04 | 2.4E−10 |

BA252 binding to LILRA and LILRB family members was then compared to commercially available anti-ILT2 antibodies 1Q-G2 (Creative, #DCABH-4494), VMP55 (Novus, #NBP2-50475PE), 4F9 (Biorad, #MCA2515F), 292305 (R&D Systems, #MAB20171), 238145 (Abcam, #EPR22861-6), HP-F1 (Invitrogen, #16-5129-82) and 15G8 (see Table 2) by SPR. Irrelevant antibody VRC01 IgG1 N297A was used as a negative isotype control.

Results are shown in Table 9. All anti-ILT2 antibodies bound to ILT2 with measurable affinities ranging from 0.15 nM to 10 nM. Anti-ILT2 antibodies 15G8, 1Q-G2, VMP55, 292305, 4F9 and 238145 also showed measurable binding to LILRA1 and ILT6. Conversely, BA252 did not bind to LILRA1 and showed only very weak interaction with ILT6.

None of the anti-ILT2 clones tested bound to LILRA2, LILRA4, LILRA5, LILRA6, LILRB3, LILRB4, or LILRB5.

TABLE 9
Anti-ILT2 Antibody Binding Affinity to LILRA & LILRB family Members.

| | Antigen | | | | | | | | | | | | |
|---|---|---|---|---|---|---|---|---|---|---|---|---|---|
| Analyte | ILT2 | | LILRB3 | LILRB4 | LILRB5 | LILRA1 | | LILRA2 | ILT6 | | LILRA4 | LILRA5 | LILRA6 |
| BA252 | 0.20 | nM | NBD | NBD | NBD | NBD | | NBD | >1000 | nM | NBD | NBD | NBD |
| 15G8 | 10 | nM | NBD | NBD | NBD | 21 | nM | NBD | 24 | nM | NBD | NBD | NBD |
| 1Q-G2 | 1.7 | nM | NBD | NBD | NBD | 5 | nM | NBD | 13 | nM | NBD | NBD | NBD |
| VMP55 | 0.15 | nM | NBD | NBD | NBD | 32 | nM | NBD | 6 | nM | NBD | NBD | NBD |
| 292305 | 2.4 | nM | NBD | NBD | NBD | 260 | nM | NBD | 100 | nM | NBD | NBD | NBD |
| 4F9 | 0.89 | nM | NBD | NBD | NBD | 40 | nM | NBD | 26 | nM | NBD | NBD | NBD |
| 238145 | 0.24 | nM | NBD | NBD | NBD | 0.81 | nM | NBD | <0.1 | nM | NBD | NBD | NBD |
| HP-F1 | 3.6 | nM | NBD | NBD | NBD | 7.2 | nM | NBD | 15 | nM | NBD | NBD | NBD |

| | | | | | | | | | | |
|---|---|---|---|---|---|---|---|---|---|---|
| Control | NBD | NBD | NBD | NBD | NBD | NBD | NBD | NBD | NBD | NBD |

NBD = no binding detected

### Binding to Cells Expressing Human ILT2

The capacity of anti-ILT2 antibodies to bind to cells expressing human ILT2 was tested in a variety of cell types.

Anti-ILT2 antibody variants with IgG1 N297A heavy chain constant regions (BA211, BA212, BA216, BA213, BA214 and BA215) were assessed for their ability to bind to human ILT2 expressed on the surface of CHO cells. Briefly, CHO cells were transfected with a vector encoding human ILT2 extracellular and transmembrane domains. A clone stably expressing a relatively low level of ILT2 was cultured in Power CHO-2 medium containing 4 mM L-Glutamine, 100 U/mL Penicillin, 100 μg/mL Streptomycin, 1× HT-Supplement.

For the antibody binding assay, the cells were seeded in a 96-well U-bottom tissue culture plate at a density of 3×10⁵ cells per well in 25 μL of PBS supplemented with 0.5% BSA, 1 mM EDTA and 0.05% sodium azide (FACS Buffer). The cells were incubated for 30 minutes at 4° C. with 25 μL of a series dilution of anti-ILT2 antibodies or isotype control, at final concentrations ranging from 10 μg/mL to 0.2 ng/mL in FACS Buffer.

For antibody staining, the cells were washed twice with cold FACS Buffer and resuspended in 100 μL of FACS Buffer containing a R-Phycoerythrin goat anti-human IgG antibody (Jackson Immunoresearch/109-116-098) at a final dilution of 1/800. After a 30-minute incubation in the dark at 4° C., the cells were washed twice with cold FACS Buffer, and the cells were analyzed by flow cytometry (BD LSR Fortessa Flow Cytometer). CHO cells were identified using a plot of forward scatter-area (FSC-A) versus side scatter area (SSC-A) and another plot of FSC-A versus forward scatter-height (FSC-H) for selection of single cells. Samples were analyzed by sequentially gating on the single cell population and plotting histograms representing the fluorescence in the PE channel. For each sample, mean fluorescence intensity (MFI) was calculated, and the data were plotted by GraphPad Prism software.

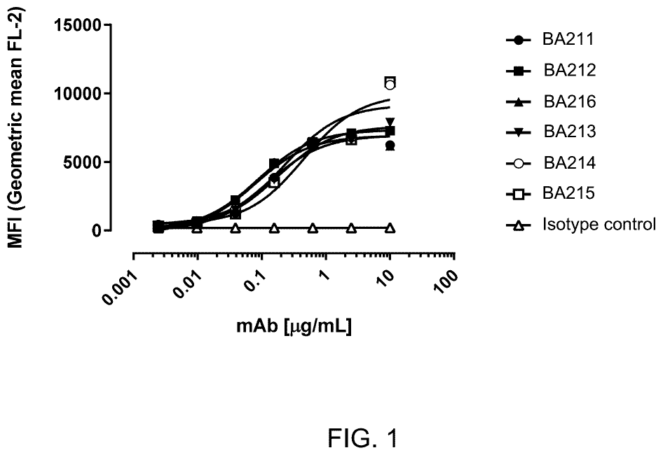

As shown in FIG. 1, BA211 and sequence optimized variants BA212, BA216, BA213, BA214, and BA215 all dose-dependently bound to CHO cells expressing relatively low levels of human ILT2. The calculated geomean area under the curve (AUC) and EC50 values are listed in Table 10.

TABLE 10

| Anti-ILT2 antibodies binding to ILT2- | | |
|---|---|---|
| expressing CHO cells. | | |
| Antibody | AUC | EC₅₀ (μg/ml) |
| BA211 | 13342 | 0.123 |
| BA212 | 15132 | 0.087 |
| BA216 | 14407 | 0.078 |
| BA213 | 13650 | 0.175 |
| BA214 | 15477 | 0.231 |
| BA215 | 14278 | 0.449 |

The same anti-ILT2 antibody variants were tested for their ability to bind to human ILT2 expressed on the surface of Jurkat cells. Briefly, Jurkat cells were transfected with a vector encoding full-length ILT2, and a clone stably expressing ILT2 was selected. This stable cell line, which also expressed a luciferase reporter gene under NFAT response elements and FcgRIIIa (Promega/G7102), was cultured in RPMI-1640 medium supplemented with 10% heat-inactivated FBS, 1% non-essential amino acids, 250 μg/mL G418 disulfate salt solution, 100 μg/mL hygromycin and 1 mM sodium pyruvate.

For the antibody binding assay, the cells were seeded in a 96-well U-bottom tissue culture plate at a density of 3×10⁵ cells per well in 25 μL of PBS supplemented with 0.5% BSA, 1 mM EDTA and 0.05% sodium azide (FACS Buffer). The cells were incubated for 30 minutes at 4° C. with 25 L of a series dilution of anti-ILT2 antibodies, or isotype control antibody (VRC01 IgG₁) at concentrations ranging from 10 μg/mL to 2.4 ng/ml in FACS Buffer. Cells were stained and identified as above.

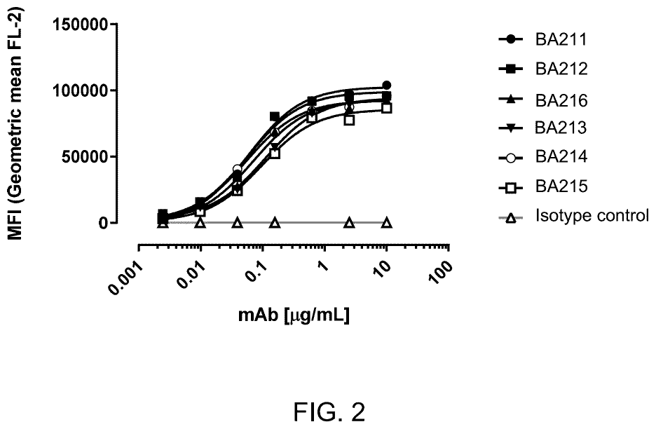

As shown in FIG. 2, anti-ILT2 antibody BA211 and sequence optimized variants BA212, BA216, BA213, BA214 and BA215 dose-dependently bound to Jurkat cells expressing human ILT2. Results are listed in Table 11.

TABLE 11

| Anti-ILT2 antibodies binding to ILT2-expressing Jurkat cells. | | |
|---|---|---|
| Antibody | AUC | EC₅₀ (μg/ml) |
| BA211 | 225681 | 0.059 |
| BA212 | 222439 | 0.057 |
| BA216 | 198758 | 0.070 |
| BA213 | 189474 | 0.110 |
| BA214 | 208116 | 0.055 |
| BA215 | 172820 | 0.096 |

The ability of anti-ILT2 sequence-optimized variant antibodies formatted with different Fc backbones to bind to human ILT2 highly expressed on the surface of CHO cells was assessed. Briefly, CHO cells were transfected with a vector encoding human ILT2 extracellular and transmembrane domains. A clone expressing relatively high levels of ILT2 on the membrane was selected and cultured in Power CHO-2 medium containing 4 mM L-Glutamine, 100 U/mL Penicillin, 100 μg/mL Streptomycin, 1× HT-Supplement.

For the antibody binding assay, the cells were seeded in a 96-well U-bottom tissue culture plate at a density of 3×10⁵ cells per well in 25 μL of PBS supplemented with 0.5% BSA, 1 mM EDTA and 0.05% sodium azide (FACS Buffer). The cells were incubated for 30 minutes at 4° C. with 25 L of a series dilution of anti-ILT2 antibodies or isotype control at concentrations from 10 μg/mL to 2.4 ng/mL in FACS Buffer.

For antibody staining, the cells were washed twice with cold FACS Buffer and resuspended in FACS Buffer containing R-Phycoerythrin goat anti-human IgG (Fab′) 2 (Jackson ImmunoResearch/109-116-097) at 1:250 dilution. After a 30-minute incubation in the dark at 4° C., the cells were washed twice with cold FACS Buffer, and the cells were analyzed by flow cytometry (BD LSR Fortessa Flow Cytometer). CHO-ILT2 cells were identified and MFI was calculated as above.

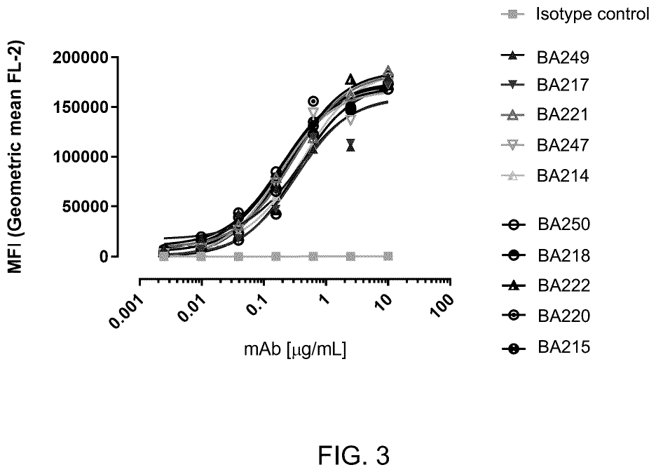

As shown in FIG. 3, all sequence optimized anti-ILT2 antibodies bound to CHO cells expressing high levels of human ILT2 in a dose-dependent manner. The calculated AUC and EC50 values are shown in Table 12.

TABLE 12
Anti-ILT2 antibodies binding to ILT2-expressing CHO cells.

| Antibody | AUC | 95% CI | EC₅₀ (μg/mL) | 95% CI (μg/mL) |
|---|---|---|---|---|
| BA214 | 379740 | 200212-720249 | 0.104 | 0.005-2.105 |
| BA215 | 392229 | 227748-675500 | 0.082 | 0.014-0.478 |
| BA217 | 264082 | 200916-347107 | 0.248 | 0.094-0.655 |
| BA218 | 294751 | 209658-414382 | 0.227 | 0.084-0.613 |
| BA219 | 307176 | 251396-375333 | 0.183 | 0.077-0.432 |
| BA220 | 304607 | 209658-414382 | 0.282 | 0.045-1.776 |
| BA221 | 360571 | 244693-531325 | 0.141 | 0.027-0.722 |
| BA222 | 372656 | 245051-566709 | 0.115 | 0.018-0.728 |
| BA249 | 349259 | 184767-660192 | 0.117 | 0.0009-1.457 |
| BA250 | 383800 | 245703-599514 | 0.087 | 0.016-0.490 |

Anti-ILT2 antibody variant BA252 was compared with reference anti-ILT2 antibody 15G8 (see Table 2) for binding to cells expressing various levels of human ILT2.

CHO cells expressing relatively high and low levels of human ILT2 were seeded in a 96-well U-bottom tissue culture plate at a density of 2×10⁵ cells per well in 50 μL of PBS supplemented with 0.5% BSA, 1 mM EDTA and 0.05% sodium azide (FACS Buffer). The cells were incubated for 30 minutes at 4° C. with 50 μL of a series dilution of anti-ILT2 antibodies or isotype control (VRC01 IgG₄) at concentrations from 10 μg/mL to 0.2 ng/mL in FACS Buffer.

For antibody staining, the cells were washed three times with cold FACS Buffer and resuspended in FACS Buffer containing a FITC-conjugated mouse anti-human IgG₄ antibody (Southern Biotech/9190-02) at a final concentration of 1.25 g/mL. After a 30-minute incubation in the dark at 4° C., the cells were washed three times and analyzed as above. Statistical analysis was done by a comparison of best-fit values between BA252 and 15G8 data sets using an ‘extra sum-of-squares F test’.

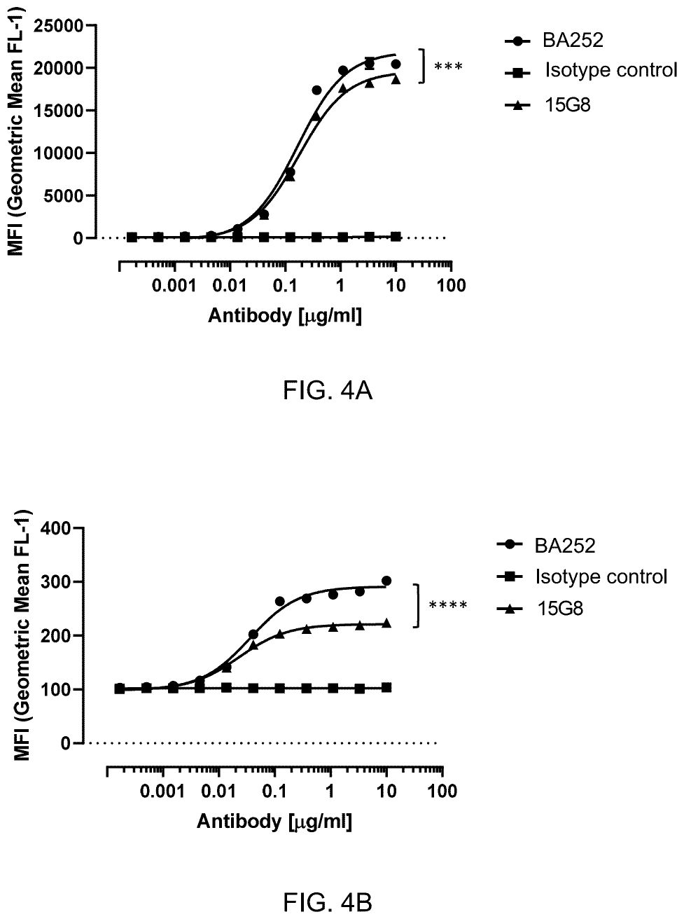

As shown in FIGS. 4A and 4B, BA252 and 15G8 dose-dependently bound to CHO cells expressing human ILT2 at relatively high and low levels, with BA252 exhibiting significantly higher maximum binding levels (Bmax). The calculated AUC and EC50 values are listed in Tables 13 and 14.

TABLE 13
Anti-ILT2 antibodies binding to ILT2 high-
expressing CHO cells.

| | | | EC₅₀ | 95% CI |
|---|---|---|---|---|
| Antibody | AUC | 95% CI | (μg/ml) | (μg/ml) |
| BA252 | 43063 | 31175-59485 | 0.165 | 0.105-0.261 |
| 15G8 | 38837 | 26258-57443 | 0.155 | 0.116-0.208 |
| Isotype | 570 | 427-762 | ND | ND |

TABLE 14
Anti-ILT2 antibodies binding to ILT2
low-expressing CHO cells.

| Antibody | AUC | 95% CI | EC₅₀ (μg/ml) | 95% CI (μg/ml) |
|---|---|---|---|---|
| BA252 | 1068 | 746-1527 | 0.032 | 0.02-0.05 |
| 15G8 | 922 | 605-1403 | 0.019 | 0.013-0.03 |
| Isotype | 542 | 391-752 | ND | ND |

### Example 2: Anti-ILT2 Antibodies Block Ligand Binding to ILT2

### Anti-ILT2 Antibodies BA211 and BA252 Block Binding of Non-Classical Major Histocompatibility Class (MHC) I Molecule HLA-G to Human ILT2 Expressed on CHO Cells

Anti-ILT2 antibodies were tested in their ability to block binding between ILT2 and non-classical MHC I ligand, HLA-G.

For the blocking assay, 25 μL of CHO cells expressing ILT2 at a relatively high level were dispensed into a 96-well U-bottom microtiter plate at a final concentration of 2×10⁵ cells per well. 25 μL of a series dilution of anti-ILT2 antibody BA211, commercial anti-ILT2 antibody HP-F1 (Thermofisher/16-5129-82) or isotype control (VRC01 IgG₁) in FACS buffer were added to the wells in order to get final assay concentrations ranging from 10 μg/mL to 2.4 ng/ml. The mixture was incubated 30 minutes at 4° C. The ILT2 ligand HLA-G-Fc, was conjugated with R-Phycoerythrin (R-PE) using the LYNX Rapid R-PE Antibody Conjugation Kit (Bio-Rad/LNK022RPE). HLA-G-Fc-PE was resuspended at 1 μg/mL in FACS buffer and 50 μL of the solution were added to wells of a 96-well U-bottom tissue culture plate. Following a 30-minute incubation in the dark at 4° C., cells were washed by the addition of cold FACS buffer. This wash was repeated twice before analysis of the cell fluorescence by flow cytometry (BD LSR Fortessa Flow Cytometer).

CHO cells were identified using a plot of forward scatter-area (FSC-A) versus side scatter area (SSC-A) and another plot of FSC-A versus side scatter height (FSC-H) for selection of single cells. Samples were analyzed by sequentially gating on the single cell population and plotting histograms representing the fluorescence in the PE channel. For each antibody sample, percent binding was calculated as: (MFI (sample)-MFI (no HLA-G-PE^{background}))/(MFI (no antibody^{full binding})-MFI (no HLA-G-PE^{background}))*100. Data was plotted using GraphPad Prism.

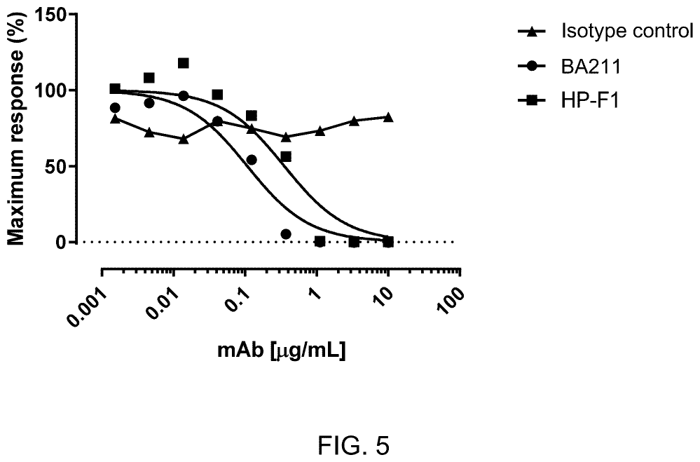

As shown in FIG. 5, BA211 and HP-F1 blocked binding of HLA-G-Fc to ILT2-expressing cells. In this assay, the calculated IC₅₀ values for BA211 and HP-F1 were 0.159 μg/mL (95% CI 0.099-0.260 μg/mL, N=8) and 0.307 μg/mL (95% CI 0.176-0.530 μg/mL, N=8), respectively.

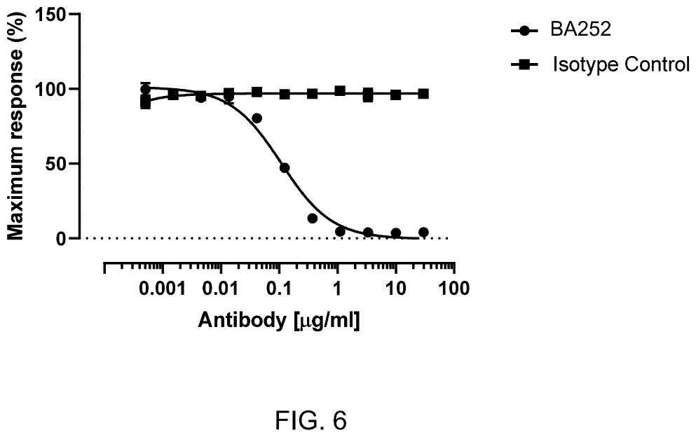

In a similar experiment, anti-ILT2 antibody BA252 blocked binding of HLA-G-Fc to ILT2-expressing CHO cells. FIG. 6 shows BA252 compared with isotype control antibody VRC01 IgG₄. The calculated IC₅₀ value for BA252 was 0.103 μg/mL (95% CI 0.077-0.138 μg/mL, N=3), demonstrating high potency inhibition of ligand binding.

### Anti-ILT2 Antibody BA252 Blocks Binding of Classical MHC I Molecules HLA-A, HLA-B and HLA-C to CHO Cells Expressing Human ILT2

BA252 was tested for the ability to block binding between ILT2 and classical MHC I molecule ligands.

Phycoerythrin (PE)-conjugated HLA-A*02:01 pentamer (ProImmune/F008-2A-G), HLA-B*07:02 pentamer (ProImmune/F045-2A-G) and HLA-C*07:02 pentamer (ProImmune/F3269-2A-G) were suspended at 0.8 μg/mL, 0.4 μg/mL and 0.4 μg/mL respectively in FACS buffer and 50 μL of each solution were added to separate wells of three 96-well U-bottom tissue culture plates. Twenty-five μL of a series dilution of BA252 or isotype control antibody VRC01 IgG₄ in FACS buffer were added to the wells in order to get final assay concentrations ranging from 30 μg/mL to 0.5 ng/mL. CHO cells expressing ILT2 at a relatively high level were then added in 25 μL at a final concentration of 2×10⁵ cells per well. Following a 45-minute incubation in the dark at 4° C., cells were washed by the addition of cold FACS buffer. This wash was repeated twice before analysis of the cell fluorescence by flow cytometry (BD LSR Fortessa Flow Cytometer). CHO cells were identified and samples were analyzed as above.

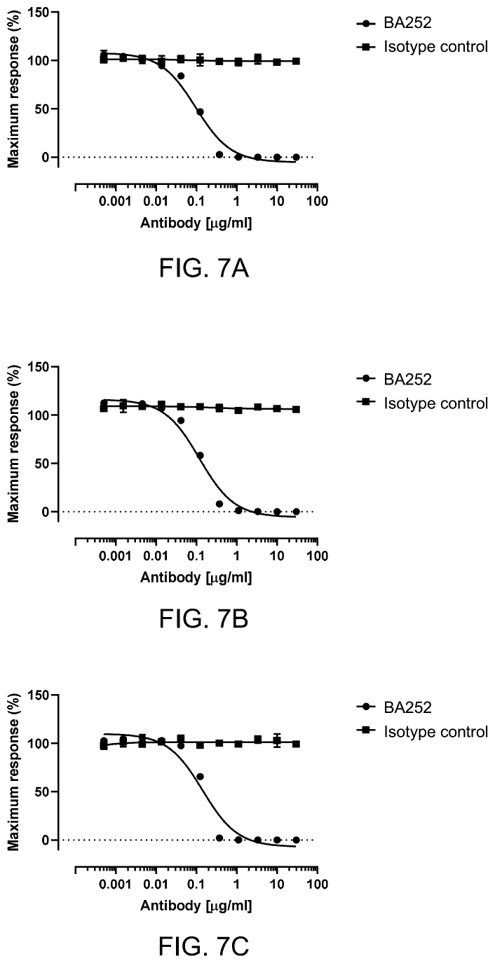

As shown in FIGS. 7A-7C, BA252 blocked binding of HLA-A*02:01, HLA-B*07:02, and HLA-C*07:02 pentamers to ILT2-expressing cells at IC₅₀ values of 0.110 μg/mL (SD+/−0.020 μg/mL, N=2), 0.136 μg/mL (SD+/−0.025 μg/mL, N=2), and 0.158 μg/mL (SD+/−0.022 μg/mL, N=2), respectively.

### Example 3: Functionality of Anti-ILT2 Antibodies

### Sequence Optimized Anti-ILT2 Antibodies Block ILT2 and Increase FcγRIIIa-NFAT Signaling in a Human ILT2 T Cell Reporter Assay

In this example, anti-ILT2 antibodies BA214, BA221, BA251, and BA252 were tested for their ability to block binding between ILT2-expressing Jurkat reporter cells and HLA-G-expressing Ramos target cells, and enhance FcγRIIIa signaling in Jurkat cells through NFAT. The commercial anti-ILT2 antibody HP-F1 was used as a positive control, and VRC01 with IgG₁ N297A and IgG₄ S228P constant regions as negative isotype controls.

Jurkat cells (Promega/G7102) expressing FcγRIIIa and an NFAT-Luciferase reporter gene were engineered in-house to express ILT2 and were used to model effector immune cells. These effector cells were expanded in RPMI 1640 medium containing 10% fetal bovine serum, 1% MEM non-essential amino acids, 500 μg/mL G418, 200 μg/mL Hygromycin B and 1 mM sodium pyruvate. In the co-culture reporter assay, target cells consisted of Ramos cells engineered in-house to express HLA-G. The medium for the expansion of target cells and for the co-culture reporter assay was made of RPMI 1640 medium supplemented with 20% fetal bovine serum, 1 mM Sodium Pyruvate, 10 mM Hepes and 50 μM β-mercapto-ethanol.

Jurkat effector cells were seeded at 1×10⁵ in U-shape wells of a 96-well microtiter plate. An anti-CD20 antibody, rituximab (Genentech/NDC Code 50242-053-06), was used as an opsonizing agent, binding to CD20 on Ramos target cells and FcγRIIIa on Jurkat effector cells. Rituximab, used at 10 μg/mL final assay concentration, was pre-incubated for 30 minutes with Jurkat effector cells at 37° C. and 5% CO₂. Dilution series of anti-ILT2 antibodies ranging from 10 μg/mL to 2.4 ng/ml final assay concentration were then added to relevant wells, as well as 5×10⁴ Ramos target cells. The final assay volume was 100 μL. Plates were incubated overnight at 37° C. and 5% CO₂. To measure luciferase reporter gene expression, the medium in each well was mixed and 60 μL were transferred to a new 96-well flat bottom microtiter plate. Sixty μL of Bio-Glo luciferase substrate (Promega/G7941) were added to each well. The luminescence signal (RLU) was measured using a Tecan Infinite M1000-Pro plate reader. RLU values were plotted against anti-ILT2 antibody concentrations using GraphPad Prism software and EC50 values were calculated.

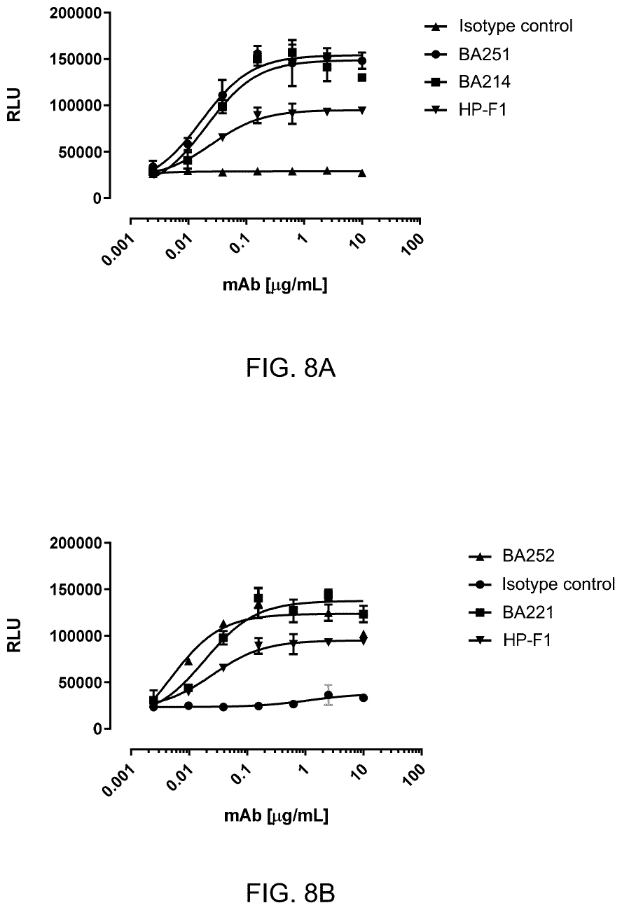

As shown in FIGS. 8A-8B, all anti-ILT2 antibodies increased FcγRIIIa NFAT signaling relative to isotype controls in human ILT2-expressing Jurkat effector cells. As the luminescence signal varied across the 3 independent experiments, AUC values for BA214, BA221, BA251 and BA252 were normalized to the AUC of benchmark anti-ILT2 antibody HP-F1. Antibodies BA214, BA251, BA221, and BA251 induced a larger increased in luminescence than commercial anti-ILT2 antibody HP-F1. The calculated normalized AUC and geomean EC50 values are listed in Table 15.

TABLE 15
Anti-ILT2 antibodies effect on FcγRIIIa-NFAT signaling.

| | Average | | EC₅₀ | 95% CI |
|---|---|---|---|---|
| Antibody | AUC | SD | (mg/mL) | (mg/mL) |
| BA214 | 145.22 | 17.77 | 0.0340 | 0.0005-0.245 |
| BA251 | 159.24 | 10.77 | 0.0212 | 0.004-0.108 |
| BA221 | 143.09 | 20.17 | 0.0328 | 0.0076-0.142 |
| BA252 | 158.58 | 27.18 | 0.0153 | 0.0003-0.763 |
| HP-F1 | 100 | 0 | 0.0328 | 0.0191-0.057 |

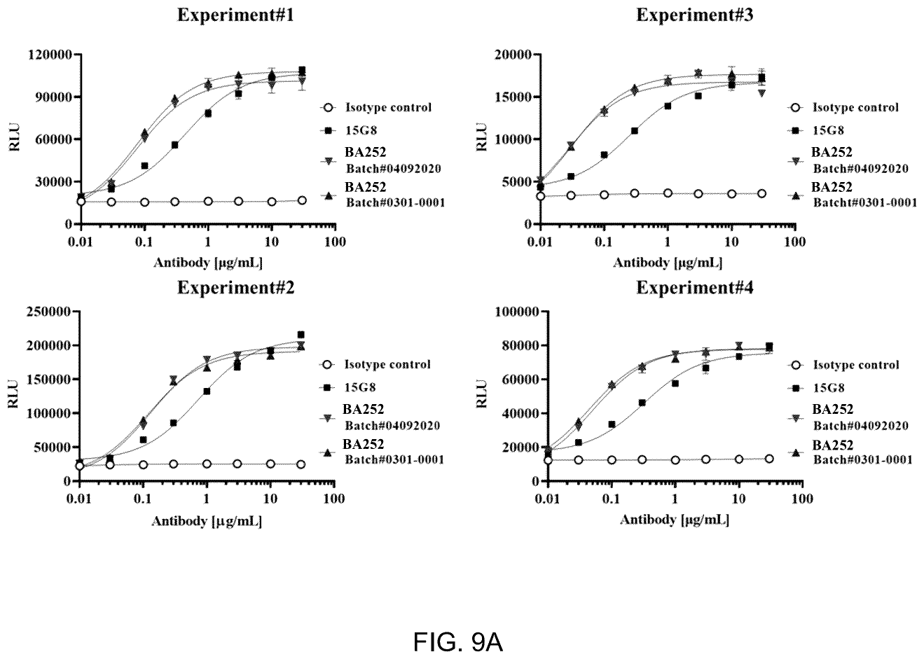

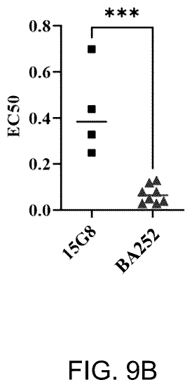

In a similar experiment, anti-ILT2 antibody BA252 was compared to reference antibody 15G8. As shown in FIGS. 9A-9B, BA252 increased FcγRIIIa-NFAT signaling, relative to isotype control, showing superior potency compared to 15G8. The calculated EC50 values for BA252 and 15G8 were 0.07±0.04 μg/mL and 0.43±0.20 μg/mL, respectively.

Anti-ILT2 Antibody BA252 does not Bind C1q

Anti-ILT2 antibody variant BA252 was assessed for binding to complement component 1q (C1q) protein, the requisite first step in the classical complement cascade for the induction of complement-dependent cytotoxicity (CDC).

C1q binding was determined using an enzyme linked immunosorbent assay (ELISA). Briefly, 96-well NUNC Maxisorp plates (Thermoscientific/44-2404-21) were coated overnight at 4° C. with 50 μL of a series dilution (300-0.14 μg/mL) of either BA252 or control antibodies nivolumab (Evidentric GmbH), an anti-PD-1 IgG₄ antibody, adalimumab (Myonex), an anti-TNFα IgG₁ antibody, or IgG₄ isotype control (CrownBio/C00045-5). Plates were then decanted and blocked for 1 hour at room temperature with PBS containing 2% dry milk. The binding assay was then carried out by removing the blocking solution and adding 50 μL of a 2 μg/mL solution of complement component C1q (Sigma/C1740-1 mg). The plate was washed 3 times in PBS containing 0.05% Tween 20. Bound C1q was detected by adding 50 μL of a biotinylated anti-C1q antibody (Invitrogen/MA1-40312) used at 0.2 μg/mL. Following incubation for 1 hour at room temperature, the plate was decanted and washed 3 times in PBS/0.05% Tween 20. Fifty μL of a 1.5 μg/mL solution of streptavidin-horseradish peroxidase (Jackson ImmunoResearch/016-030-084) were added to each well and the plate was incubated 1 hour at room temperature. Lastly, following plate washing in PBS/0.05% Tween 20×3 times, 50 μL of chromogen 3,3′,5,5′-tetramethylbenzidine (Invitrogen/00-4201-56) were added to each well. The plate was allowed to develop color for about 1 minute before the enzymatic reaction was stopped by adding 50 μL of a 1M HCl solution. Optical density (OD) at 450 nm was measured for each well using a Tecan Infinite M1000-Pro plate reader.

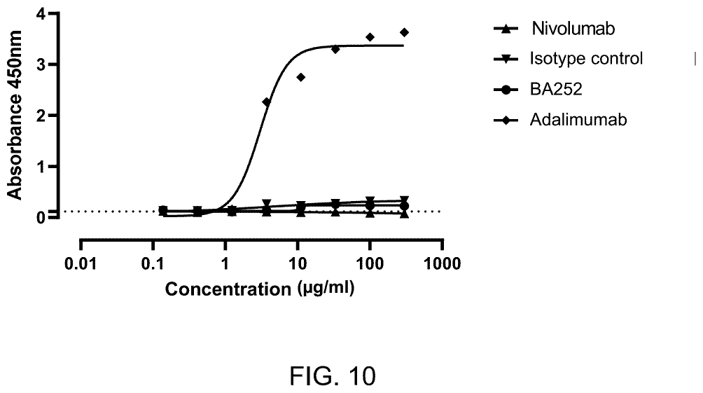

As shown in FIG. 10, BA252 demonstrated minimal binding to C1q, which was also comparable to control antibody nivolumab and human IgG4 isotype control. As a positive control, adalimumab, which is an IgG1 antibody, demonstrated C1q binding. The calculated AUC values are listed in Table 16.

TABLE 16
AUC values for anti-ILT2 antibodies.

| Antibody | Average AUC | SD |
|---|---|---|
| BA252 | 0.55 | 0.09 |
| Nivolumab | 0.52 | 0.18 |
| IgG4 Isotype control | 0.50 | 0.21 |
| Adalimumab | 6.54 | 0.79 |

### Anti-ILT2 Antibody BA252 Potentiates Primary Human T, NK and NKT Cell Activation

The potential of BA252 to promote primary immune cell functional activity was assessed in ex vivo experiments using co-cultures of peripheral blood mononuclear cells (PBMCs) and tumor cell lines.

Pharmacodynamic effects were assessed by priming PBMCs from healthy donors overnight with IL-2 and IL-15, in the presence of BA252, 15G8, or isotype control. The next day, JEG-3^{HLA-A2} cancer cells expressing HLA-G and HLA-A2 were added to the primed PBMCs at a tumor cell to PBMC ratio of 1:40 and co-cultured for 72 hr. Activation of T, NKT and NK cells was assessed by profiling of CD25 expression on T cells (CD3+CD4+ and CD3+CD8+), NKT (CD3+CD56+) and NK (CD3-CD56+) cells by flow cytometry. PBMC donors were assigned to “Responders” group if the following criteria was fulfilled: at least one of analyzed immune subpopulations (NK, NKT, CD8+ T cells) responded to BA252 by ≥20% increase of CD25 surface expression as compared to isotype control antibody. Otherwise, they were placed into the “Non-Responders” group.

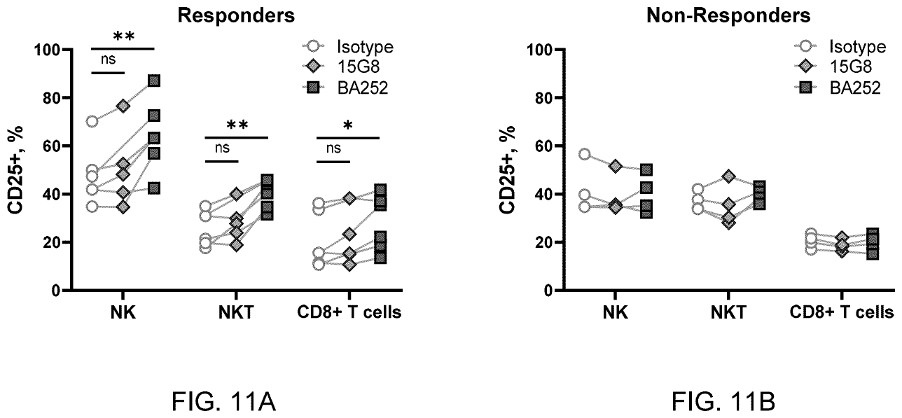

Results showing the superiority of BA252 over 15G8 in promoting primary immune cell functional activity are shown in FIGS. 11A-11B.

### BA252-Mediated ILT2 Blockade Modulates Phenotype of Primary Human Macrophages

The potency of BA252 to modulate functional activity of myeloid cells was assessed in vitro using primary human macrophages (Mφ).

Mφ were differentiated from a purified monocyte fraction of PBMCs in the presence of BA252, 15G8, or IgG₄ isotype control. Mφ were polarized in vitro into M2-like Mφ or left non-stimulated. To induce pro-tumorigenic M2 phenotype, Mφ were treated with IL-10+TGFβ cocktail or with conditioned media from JEG-3 cancer cells expressing HLA-G (Cancer cell CM). Mφ phenotype was assessed by profiling surface expression of CD86, CD163 and CD206 on CD33+ myeloid cells by flow cytometry. Statistical analysis was performed by paired t-test.

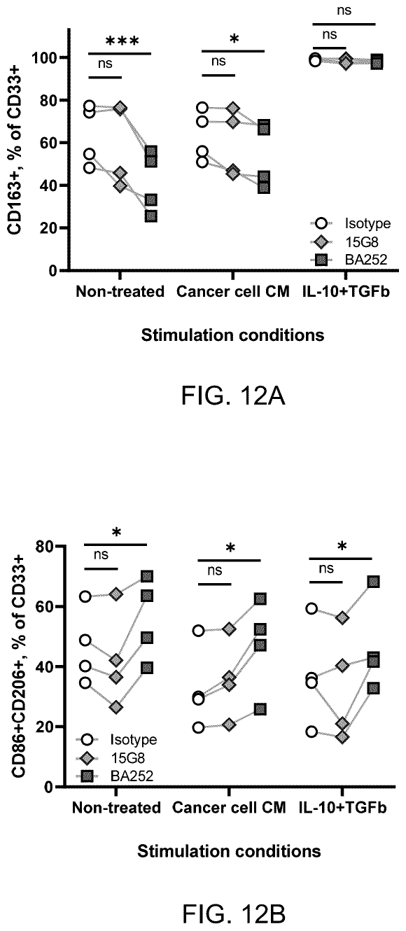

Results demonstrating the superiority of BA252 over 15G8 in suppressing M2-like and potentiating antigen-presenting phenotypes are shown in FIGS. 12A-12B.

### Example 4: Cross-Competition Binding

BA252 does not Compete with Commercial Anti-ILT2 Antibodies for Binding to Human ILT2-Expressing Cells

The ability of BA252 to block binding of anti-ILT2 antibodies 1Q-G2 (Creative, #DCABH-4494, APC-conjugated), VMP55 (Novus, #NBP2-50475PE, PE-conjugated), 4F9 (Biorad, #MCA2515F, FITC-conjugated), 292305 (R&D Systems, #FAB20171P-100, PE-conjugated) and GHI/75 (Biolegend, #333708, PE-conjugated) to human ILT2 expressed on cells was evaluated by flow cytometry.

For the epitope competition assay, CHO cells expressing ILT2 were resuspended at 8×10⁶ cells per mL in FACS buffer and 25 L were added to the wells of a U-bottom 96-well microtiter plate giving a final cell concentration of 2×10⁵ cells per well. Twenty-five μL of a series dilution of unconjugated BA252 in FACS buffer were added to the wells in order to get final assay concentrations ranging from 100 μg/mL to 0.01 μg/mL. Following a 1-hour pre-incubation on ice, 50 μL of a second anti-ILT2 antibody conjugated to a fluorochrome were added to the wells. The final assay concentrations for antibodies 1Q-G2, VMP55, 4F9, 292305 and GHI/75 were 0.2 μg/mL, 1.7 μg/mL, 2.7 μg/mL, 12.5 μg/mL and 60 μg/mL, respectively, based on pre-established titration curves. As a positive control to demonstrate epitope competition, APC-conjugated BA252 was also included and used at 0.3 μg/mL final concentration. Following another 1-hour incubation on ice in the dark, the cells were washed by centrifugation at 300 g for 5 minutes and addition of 200 μL cold FACS buffer. This wash was repeated once before analysis of the cell fluorescence by flow cytometry (BD LSR Fortessa Flow Cytometer).

CHO cells were identified using a plot of forward scatter-area (FSC-A) versus side scatter area (SSC-A) and another plot of SSC-A versus side scatter height (SSC-H) for selection of single cells. Samples were analyzed by sequentially gating on the single cell population and plotting histograms representing the fluorescence in either the FITC, PE or APC channel depending on the fluorochrome used for the secondary antibody. The data was plotted using GraphPad Prism.

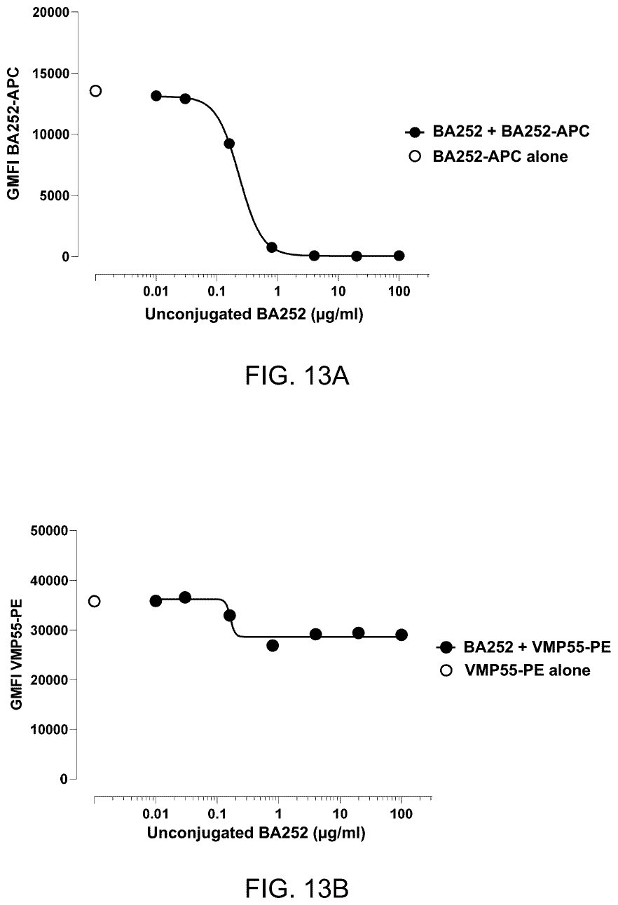

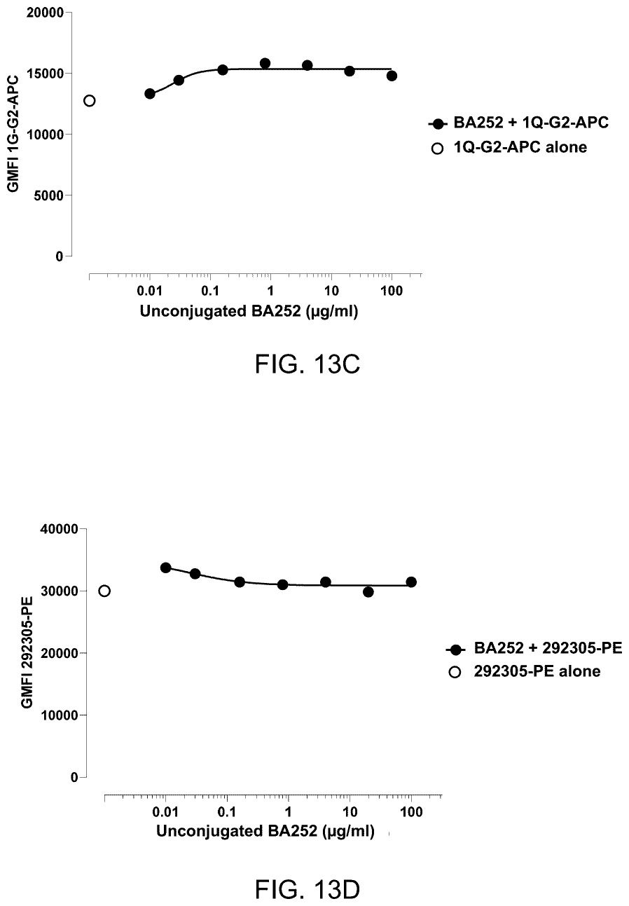

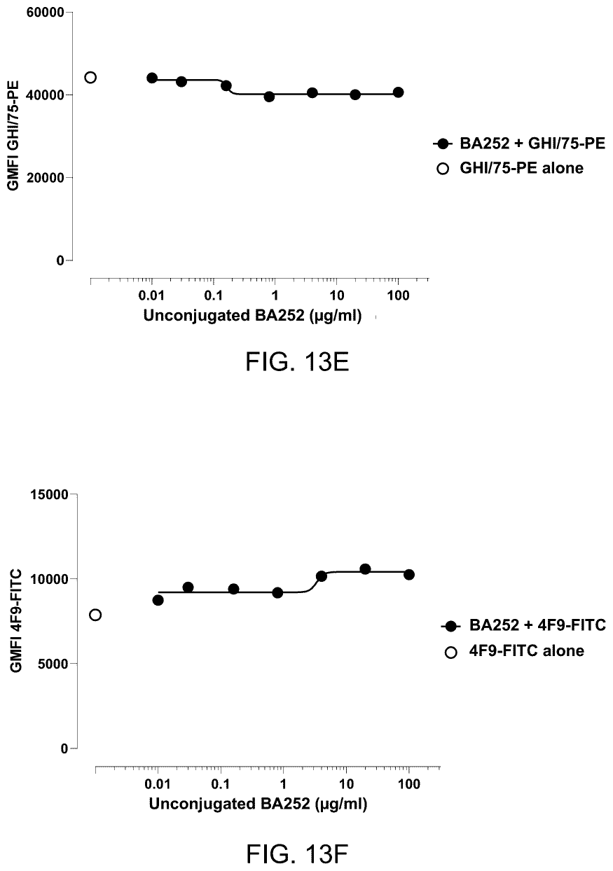

As shown in FIG. 13A, unconjugated BA252 competed with the binding of APC-conjugated BA252. BA252 did not block the binding of commercially available anti-ILT2 clones 1Q-G2, VMP55, 4F9, 292305, and GHI/75, indicating that it binds to a different epitope on human ILT2 (FIGS. 13B-13F).

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

1. An antibody that specifically binds human ILT2, the antibody comprising: a heavy chain variable region (VH) comprising the complementarity determining regions (CDRs) CDRH1, CDRH2, and CDRH3 amino acid sequences of the VH amino acid sequence set forth in SEQ ID NO: 1; and a light chain variable region (VL) comprising the CDRL1, CDRL2, and CDRL3 amino acid sequences of the VL amino acid sequence set forth in SEQ ID NO: 8.

2. The antibody of claim 1, wherein the antibody comprises the CDRH1, CDRH2, and CDRH3 amino acid sequences, respectively, set forth in SEQ ID NOs: 9, 10, and 11.

3. The antibody of claim 1, wherein the antibody comprises the CDRL1, CDRL2, and CDRL3 amino acid sequences, respectively, set forth in SEQ ID NOs: 12, 16, and 17; 13, 16, and 17; 14, 16, and 17:12, 16, and 18; 13, 16, and 18; 14, 16, and 18; or 15, 16, and 19.

4. (canceled)

5. The antibody of claim 1, wherein the antibody comprises the CDRH1, CDRH2, CDRH3, CDRL1, CDRL2, and CDRL3 amino acid sequences, respectively, set forth in SEQ ID NOs: 9, 10, 11, 12, 16, and 17; 9, 10, 11, 13, 16, and 17; 9, 10, 11, 14, 16, and 17; 9, 10, 11, 12, 16, and 18; 9, 10, 11, 13, 16, and 18; 9, 10, 11, 14, 16, and 18; or 9, 10, 11, 13, 16, and 18.

6. (canceled)

7. The antibody of claim 1, wherein the antibody comprises the VH amino acid sequence of SEQ ID NO: 1.

8. The antibody of claim 1, wherein the antibody comprises a heavy chain constant region, optionally selected from the group consisting of human IgG₁, IgG₂, IgG₃, IgG₄, IgA₁, IgA₂, and IgM.

9. The antibody of claim 8, wherein the heavy chain constant region is a human IgG₄ heavy chain constant region comprising a P at position 228, numbered according to the EU numbering system.

10. The antibody of claim 1, wherein the antibody comprises a heavy chain constant region that is a variant of a wild-type heavy chain constant region, wherein the variant heavy chain constant region binds to an FcγR with lower affinity than the wild-type heavy chain constant region binds to the FcγR.

11. The antibody of claim 10, wherein the amino acid sequence of the heavy chain constant region comprises:
(a) one or more of the following amino acid mutations, according to the EU numbering system: L234A, L235A, L235E, N297A, N297Q, N297G, P329A, P329G, G236D, P238D, S239D, S267E, L328F, and L328E; or
(b) a set of amino acid mutations selected from the group consisting of: L234A and L235A; L234A and L235E; L234A, L235A, and L329A; or L234A, L235A, and P329G; S267E and L328F; P238D and L328E; P238D and one or more substitutions selected from the group consisting of E233D, G237D, H268D, P271G, and A330R; P238D, E233D, G237D, H268D, P271G, and A330R; G236D and S267E; S239D and S267E; V262E, S267E, and L328F; and V264E, S267E, and L328F, according to the EU numbering system.

12. The antibody of claim 1, wherein the antibody comprises a heavy chain constant region that is a variant of a wild-type heavy chain constant region, wherein the variant heavy chain constant region binds to an FcγR with higher affinity than the wild-type heavy chain constant region binds to the FcγR.

13. The antibody of claim 12, wherein the amino acid sequence of the heavy chain constant region comprises:
(a) one or more of the following amino acid mutations, according to the EU numbering system: G236A, S239D, F243L, T256A, K290A, R292P, S298A, Y300L, V305I, A330L, 1332E, E333A, K334A, A339T, and P396L; or
(b) a set of amino acid mutations selected from the group consisting of: S239D; T256A; K290A; S298A; 1332E; E333A; K334A; A339T; S239D and I332E; S239D, A330L, and I332E; S298A, E333A, and K334A; G236A, S239D, and I332E; and F243L, R292P, Y300L, V305I, and P396L, according to the EU numbering system.

14-16. (canceled)

17. The antibody of claim 1, wherein the antibody comprises the VL amino acid sequence of SEQ ID NO: 2, 3, 4, 5, 6, 7, or 8.

18. The antibody of claim 17, wherein the antibody comprises a light chain comprising the amino acid sequence of SEQ ID NO: 27, 28, 29, 30, 31, or 32.

19. The antibody of claim 1, wherein the VH and VL comprise the amino acid sequences, respectively, set forth in SEQ ID NOs: 1 and 5, 1 and 2, 1 and 3, 1 and 4, 1 and 6, or 1 and 7.

20. The antibody of claim 19, wherein the heavy chain and light chain comprise the amino acid sequences, respectively, set forth in SEQ ID NOs: 26 and 30, 26 and 27, 26 and 28, 26 and 29, 26 and 31, 26 and 32, 25 and 27, 25 and 28, 25 and 29, 25 and 30, 25 and 31, 25 and 32, 24 and 27, 24 and 28, 24 and 29, 24 and 30, 24 and 31, 24 and 32, 23 and 27, 23 and 28, 23 and 29, 23 and 30, 23 and 31, 23 and 32, 22 and 27, 22 and 28, 22 and 29, 22 and 30, 22 and 31, 22 and 32, 21 and 27, 21 and 28, 21 and 29, 21 and 30, 21 and 31, 21 and 32, 20 and 27, 20 and 28, 20 and 29, 20 and 30, 20 and 31, or 20 and 32.

21. The antibody of claim 1, wherein:
(a) the antibody blocks binding of ILT2 to HLA-G, HLA-A, HLA-B and HLA-C; and/or
(b) the antibody blocks ILT2 mediated inhibition of FcγR signaling.

22. A polypeptide comprising a VH comprising the CDRH1, CDRH2, and CDRH3 amino acid sequences of the VH amino acid sequence set forth in SEQ ID NO: 1; and/or a VL comprising the CDRL1, CDRL2, and CDRL3 amino acid sequences of the VL amino acid sequence set forth in SEQ ID NO: 8.

23-32. (canceled)

33. A polynucleotide encoding: a VH, a VL, a heavy chain, and/or a light chain of the antibody of claim 1.

34. A vector comprising the polynucleotide of claim 33.

35. A recombinant host cell comprising the polynucleotide of claim.

36. A pharmaceutical composition comprising the antibody of claim 1.

37. A method of producing an antibody, the method comprising culturing the host cell of claim 35 under suitable conditions such that the polynucleotide is expressed, and the antibody is produced.

38. A method of treating cancer in a subject, the method comprising administering to the subject an effective amount of the antibody of claim 1.

39-41. (canceled)


SEQUENCE LISTING (52 sequences)

SEQ ID NO: 1 - protein, 116 aa, synthetic construct
QVQLVQSGAEVKKPGASVKVSCKASGYTFNRYYMHWVRQAPGQGLEWMGWINPYTGGTNYAQKFQGRVTMTRDTSISTAYMELSRLRSDDTAVYYCASKQPSSDYWGQGTLVTVSS

SEQ ID NO: 2 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQASQAPWTFGQGTKVEIK

SEQ ID NO: 3 - protein, 112 aa, synthetic construct
DIVMTQSPLSLPVTPGEPASISCRSSQSLLHSNTYNYLDWYLQKPGQSPQLLIYLGSNRASGVPDRFSGSGSGTDFTLKISRVEAEDVGVYYCMQASQAPWTFGQGTKVEIK

SEQ ID NO: 4 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCLQASQAPWTFGQGTKVEIK

SEQ ID NO: 5 - protein, 112 aa, synthetic construct
DIVMTQSPLSLPVTPGEPASISCRSSQSLLHSNTYNYLDWYLQKPGQSPQLLIYLGSNRASGVPDRFSGSGSGTDFTLKISRVEAEDVGVYYCLQASQAPWTFGQGTKVEIK

SEQ ID NO: 6 - protein, 112 aa, synthetic construct
DIVMTQSPLSLPVTPGEPASISCRSSQSLLHSNYYNYLDWYLQKPGQSPQLLIYLGSNRASGVPDRFSGSGSGTDFTLKISRVEAEDVGVYYCLQASQAPWTFGQGTKVEIK

SEQ ID NO: 7 - protein, 112 aa, synthetic construct
DIVMTQSPLSLPVTPGEPASISCRSSQSLLHSNYYNYLDWYLQKPGQSPQLLIYLGSNRASGVPDRFSGSGSGTDFTLKISRVEAEDVGVYYCMQASQAPWTFGQGTKVEIK

SEQ ID NO: 8 - protein, 112 aa, synthetic construct
  VARIANT 34: note = X is G, T, or Y
  VARIANT 94: note = X is L or M
DIVMTQSPLSLPVTPGEPASISCRSSQSLLHSNXYNYLDWYLQKPGQSPQLLIYLGSNRASGVPDRFSGSGSGTDFTLKISRVEAEDVGVYYCXQASQAPWTFGQGTKVEIK

SEQ ID NO: 9 - protein, 5 aa, synthetic construct
RYYMH

SEQ ID NO: 10 - protein, 17 aa, synthetic construct
WINPYTGGTNYAQKFQG

SEQ ID NO: 11 - protein, 7 aa, synthetic construct
KQPSSDY

SEQ ID NO: 12 - protein, 16 aa, synthetic construct
RSSQSLLHSNGYNYLD

SEQ ID NO: 13 - protein, 16 aa, synthetic construct
RSSQSLLHSNTYNYLD

SEQ ID NO: 14 - protein, 16 aa, synthetic construct
RSSQSLLHSNYYNYLD

SEQ ID NO: 15 - protein, 16 aa, synthetic construct
  VARIANT 11: note = X is G, T, or Y
RSSQSLLHSNXYNYLD

SEQ ID NO: 16 - protein, 7 aa, synthetic construct
LGSNRAS

SEQ ID NO: 17 - protein, 9 aa, synthetic construct
MQASQAPWT

SEQ ID NO: 18 - protein, 9 aa, synthetic construct
LQASQAPWT

SEQ ID NO: 19 - protein, 9 aa, synthetic construct
  VARIANT 1: note = X is L or M
XQASQAPWT

SEQ ID NO: 20 - protein, 446 aa, synthetic construct
QVQLVQSGAEVKKPGASVKVSCKASGYTFNRYYMHWVRQAPGQGLEWMGWINPYTGGTNYAQKFQGRVTMTRDTSISTAYMELSRLRSDDTAVYYCASKQPSSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 446 aa, synthetic construct
QVQLVQSGAEVKKPGASVKVSCKASGYTFNRYYMHWVRQAPGQGLEWMGWINPYTGGTNYAQKFQGRVTMTRDTSISTAYMELSRLRSDDTAVYYCASKQPSSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 442 aa, synthetic construct
QVQLVQSGAEVKKPGASVKVSCKASGYTFNRYYMHWVRQAPGQGLEWMGWINPYTGGTNYAQKFQGRVTMTRDTSISTAYMELSRLRSDDTAVYYCASKQPSSDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 23 - protein, 442 aa, synthetic construct
QVQLVQSGAEVKKPGASVKVSCKASGYTFNRYYMHWVRQAPGQGLEWMGWINPYTGGTNYAQKFQGRVTMTRDTSISTAYMELSRLRSDDTAVYYCASKQPSSDYWGQGTLVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 24 - protein, 443 aa, synthetic construct
QVQLVQSGAEVKKPGASVKVSCKASGYTFNRYYMHWVRQAPGQGLEWMGWINPYTGGTNYAQKFQGRVTMTRDTSISTAYMELSRLRSDDTAVYYCASKQPSSDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 445 aa, synthetic construct
QVQLVQSGAEVKKPGASVKVSCKASGYTFNRYYMHWVRQAPGQGLEWMGWINPYTGGTNYAQKFQGRVTMTRDTSISTAYMELSRLRSDDTAVYYCASKQPSSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 442 aa, synthetic construct
QVQLVQSGAEVKKPGASVKVSCKASGYTFNRYYMHWVRQAPGQGLEWMGWINPYTGGTNYAQKFQGRVTMTRDTSISTAYMELSRLRSDDTAVYYCASKQPSSDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 27 - protein, 219 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQASQAPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 219 aa, synthetic construct
DIVMTQSPLSLPVTPGEPASISCRSSQSLLHSNTYNYLDWYLQKPGQSPQLLIYLGSNRASGVPDRFSGSGSGTDFTLKISRVEAEDVGVYYCMQASQAPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 219 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCLQASQAPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 219 aa, synthetic construct
DIVMTQSPLSLPVTPGEPASISCRSSQSLLHSNTYNYLDWYLQKPGQSPQLLIYLGSNRASGVPDRFSGSGSGTDFTLKISRVEAEDVGVYYCLQASQAPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 219 aa, synthetic construct
DIVMTQSPLSLPVTPGEPASISCRSSQSLLHSNYYNYLDWYLQKPGQSPQLLIYLGSNRASGVPDRFSGSGSGTDFTLKISRVEAEDVGVYYCLQASQAPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 219 aa, synthetic construct
DIVMTQSPLSLPVTPGEPASISCRSSQSLLHSNYYNYLDWYLQKPGQSPQLLIYLGSNRASGVPDRFSGSGSGTDFTLKISRVEAEDVGVYYCMQASQAPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 36 - protein, 326 aa, synthetic construct
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 37 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 40 - protein, 107 aa, synthetic construct [sequence omitted]

SEQ ID NO: 41 - protein, 119 aa, synthetic construct
DVQLQGSGPGLVKPSETLSLTCSVTGYSITSGYYWNWIRQFPGKKLEWMGYISYDGSNNYNPSLKNRITISRDTSKNQFSLKLNSVTAADTATYYCAHGYSYYYAMDAWGQGTSVTVSS

SEQ ID NO: 42 - protein, 106 aa, synthetic construct
DIQMTQSPSSLSASVGDRVTITCRTSQDISNYLNWYQQKPGKAVKLLISYTSRLHSGVPSRFSGSGSGTDYTLTISSLQPEDFATYYCQQGNTLPTFGQGTKLEIK

SEQ ID NO: 43 - protein, 627 aa, Homo sapiens
GHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 44 - protein, 435 aa, Homo sapiens
GHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRH

SEQ ID NO: 45 - protein, 435 aa, Homo sapiens
GHLPKPTLWAEPGSVITQGSPVTLRCQGGQETQEYRLYREKKTAPWITRIPQELVKKGQFPIPSITWEHTGRYRCYYGSDTAGRSESSDPLELVVTGAYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRH

SEQ ID NO: 46 - protein, 435 aa, Homo sapiens
GHLPKPTLWAEPGSVITQGSPVTLRCQGGQETQEYRLYREKKTAPWITRIPQELVKKGQFPIPSITWEHAGRYRCYYGSDTAGRSESSDPLELVVTGAYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRH

SEQ ID NO: 47 - protein, 418 aa, Macaca fascicularis
GILPKPTLWAEPDRVITQGSPVTLRCQGNLEAREYHLYRERKSASWITLVRPELVKKGQFPIPSTTWEHTGRYRCLYYSHSWWSEPSDPLELVVTGVYSKPTLSALPSPVVASGGNVTLQCDSQLAFGGFILCKEGDEHPQCLNSQPSTRGASRAVFSVGPVSPSRRWSYRCYGYDSRSPSVWSLPSDLLELLVPGVSKKPSLSVQPGPVVAPGEKLTLQCGSDAGYDRFVLYKEGERDFPQRSGRQPQAGLSQANFTLGPVRGSHGGQYRCSGAHNLSSEWSAPSDPLDILIAGQIRARPSLSVQPGPTVASGENVTLLCQSQGWMDTFLLTKEGAADAPLRLKSKYQSHMYQAEFPMSPVTSAHAGTYRCYASFSSNPYLLTHPSEPLELVVSGPEDQPLTPTGSAPQSGLGRHLG

SEQ ID NO: 48 - protein, 435 aa, Macaca fascicularis
GILPKPTLWAEPDRVITQGSPVTLRCQGNLEALGYHLYRERKSASWITSIRPELVRKGQFPIPSITWEDAGRYRCLYYSHSWWSEHSDPLELVVTGTYSKPTLSALPSPVVASGGNVTLQCDSQLAFGGFILCKEGDEHPQCLNSQPSTRGASRAVFSVGPVSPSRRWSYRCYGYDSHSPYVWSLPSDLLELLVPGVSNKPSLSVQPGPVVAPGEKLTLQCGSDVGYDRFVLYKEGERDLLQRSGRQPQAGLSQANFSLGPVWGSHGGQYRCSGAHNLSSEWSAPSDPLDILISRQTQARPSLSVQPGPMVASGENVTLLCQSQGWMDTFLLIKEGAADAPLRLKSKRRSYIYQAEFPMSPVTSTHAGTYRCYGSFSSNPYLLTHPSEPLELVVSGPSESPSLPLTGPTPTPGPEDQSLNPTGSDPQSGLGRHLG

SEQ ID NO: 49 - protein, 420 aa, Macaca fascicularis
GILPKPTLWAEPDRVITQGSPVTLRCQGNLEARGYHLYRERKSASWITLIRPELVKKGQFPIPSITWEHAGRYRCQYSSRNSWSEPSDPLELVVTGAYSKPTLSALPSPVVASGGSVTLQCVSQVAFDGFTLCKEGEDEHPQRLNCQSHARGWSWAVFSVGPVSPSRRWSYRCYSYDSRSPYVWSLPSDLLELLVPGVSKKPSLSVQPGPVVAPGESLTLQCGSDVGYDRFVLYKEGEGEFLQRPGRQPQAGLSQANFTLGPVRRSHGGQYRCSGAHNLSSEWSAPSDPLDILISEQFYDVVSLSVQPGPTVTSGENVTLLCQSRAQFHTFLLTREGAAHPPLHLRSEHQAQQYQAEFPMGPVTSAHAGTYRCYSSLSFNPYLLSHPSDPLELVVSGPSRSPSPPLTGPTPTPGLGRHLG

SEQ ID NO: 50 - protein, 435 aa, Macaca mulatta
GTFPKPTLWAEPGSMISKGSPVTLRCQGSLPVQDYRLQREKKTASWVRRIQQELVKKGYFAIASIAWEHTGQYRCQYYSHSWWSEPSDPLELVVTGAYSKPTLSALPSPVVASGGNVTLQCDSQVAFGGFVLCKEGEDEHPQCHSQPRTRGSSRAVFSVGPVSPSRRWSYRCYGYELHYRYVWSLPSDLLELLVPGVSKKPSLSVQPGPVVAPGDKLTLQCGSDAGYNRFALYKEGERDFLQRPGRQPQAGLSQANFLLGPVRRSHGGQYRCSGAHNLSSEWSAPSDPLDILIAGQIRGRPSLLVQPGPTVVSGENVTLLCQSSWQFHVFLLTQAGAADAHLHLRSMYKYPKYQAEFPMSPVTSAHAGTYRCYGSHSSDSYLLSIPSDPLELVVSGPSGGPSSPTTGPTSTCGPEDQPLTPTGSDPQSGLGRHLG

SEQ ID NO: 51 - protein, 436 aa, Macaca mulatta
GILPKPTLWAEPGSMISEGSPVTLRCQGSLQVQDYCLYREKKPASWVRQIRQELVKKGYFAIGFITWEHTGQYRCQYYNHSWWSEPSDPLELVVIGAYSKPTLSALPSPVVASGRNVTLQCDSQVAFDGFILCKEGEDEHPQRLNCQSHARGWSWAVFSVGPVSPSHRWSYRCYGYISSDPYVWSLPSDLLELLVPGVSKKPSLSVQPGPVVARGDKLTLQCGSDAGYDRFALYKEGEGDFLQRPGQQPQAGLSQANFLLGPVSRSHGGQYRCSGAHNLSSEWSAPSDPLDILIAGQIRGRPFLSVQPGPKVVSGENVTLLCQSSWQFHAFLLTQAGAADAHLHLRSMHKYPKYQAEFPISPVTSAHAGTYRCYGSRSSNPYLLSVPSDPLELVVSGPSGGPSSPTTGPTSTCGPEDQPLTPTGSAPQSGLGRHLG

SEQ ID NO: 52 - protein, 438 aa, Chlorocebus aethiops
GTLPKPTLWAEPGSVISEGSPVTLRCQGSLQVQEYRLYREKNPASWVRRIPQELVKKGYFPIASITWEHAGRYRCNYGSYIEGRSEPSDPLDLMVTGAYRKPTLSALPSPVVASGGNVSLQCVSQVAFDSFILCKEGEDEHPQRLNSHFHAHGWSWAVFSVGPVSPSRRWSYRCYGYDSRSPYMWSLPSDLLELLVPGVSKKPSLSVQPGPVVALGEKLTLQCGSDAGYNRFVLYKEGERDFLQRAGRQPQAGLSQANFPLGLVSRSHGGQYRCYGAHNLSSEWSSPSDPLDILISGQFYVTVSLSVLPGPMVASGENVTLLCQSWWELHTFLLTKEGAAHPPPRLRSKHRAPKYQAEFPMGPVTSAHKGTYRCYGSHSSNPYLLSLPSDPLELVVSGPSGSPSLPPTGSTPTPAGPEDQPLTPTGSDPQSGLGRHLG